SEQUENCE LISTING (59 sequences)

SEQ ID NO: 1 - DNA, 144 bp, synthetic construct
  source 1..144: mol_type = other DNA
aaagttaaacaaaattatttgtagagggaaaccgttgtggtctccctgaatatattatacgagccttatgcatgcccgtaaagttatccagcaaccactcatagacctagggcagcagatagggacgacgtggtgttagctgtg

SEQ ID NO: 2 - DNA, 95 bp, synthetic construct
  source 1..95: mol_type = other DNA
tatccatcctccatcgccacgatagttcatggcgataggtagaatagcaatgaacgattatccctatcaagcattctgactgataattgctcaca

SEQ ID NO: 3 - DNA, 28 bp, synthetic construct
  source 1..28: mol_type = other DNA
cgaattcattaaagaggagaaaggtacc

SEQ ID NO: 4 - DNA, 105 bp, synthetic construct
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 5 - DNA, 147 bp, synthetic construct
  source 1..147: mol_type = other DNA
atgaataaggcctacagtatcatttggagccactccagacaggcctggattgtggcctcagagttagccagaggacatggttttgtccttgcaaaaaatacactgctggtattggcggttgtttccacaatcggaaatgcatttgca

SEQ ID NO: 6 - DNA, 126 bp, synthetic construct
  source 1..126: mol_type = other DNA
atgcacggtaagacacaagctaccagtgggactatccagtccatgcatggaaaaacccaagcgacatctggaacgattcagagcatgcatgggaaaactcaggcaacgtcgggtacaatccaatca

SEQ ID NO: 7 - DNA, 162 bp, synthetic construct
  source 1..162: mol_type = other DNA
gtgaataacaatggaagcattgtcattaataacagcattataaacgggaatattacgaatgatgctgacttaagttttggtacagcaaagctgctctctgctacagtgaatggtagtcttgttaataacaaaaatatcattcttaatcctacaaaagaaagt

SEQ ID NO: 8 - DNA, 1323 bp, synthetic construct
  source 1..1323: mol_type = other DNA
ataggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattctctctgaagaaccgcgtagttgccggagcttatgattacacactgcagaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagctgaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctga

SEQ ID NO: 9 - DNA, 918 bp, synthetic construct
  source 1..918: mol_type = other DNA
atgaatattcgtgatcttgagtacctggtggcattggctgaacaccgccattttcggcgtgcggcagattcctgccacgttagccagccgacgcttagcgggcaaattcgtaagctggaagatgagctgggcgtgatgttgctggagcggaccagccgtaaagtgttgttcacccaggcgggaatgctgctggtggatcaggcgcgtaccgtgctgcgtgaggtgaaagtccttaaagagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 10 - DNA, 714 bp, synthetic construct
  source 1..714: mol_type = other DNA
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 11 - DNA, 603 bp, synthetic construct
  source 1..603: mol_type = other DNA
atgatcgtacaaattggtcggcgcgaagagttcgataaaaaactgctgggcgagatgcacaagttgcgtgctcaagtgttcaaggagcgcaaaggctgggacgttagtgtcatcgacgagatggaaatcgatggttatgacgcactcagtccttattacatgttgatccaggaagatactcctgaagcccaggttttcggttgctggcgaattctcgataccactggcccctacatgctgaagaacaccttcccggagcttctgcacggcaaggaagcgccttgctcgccgcacatctgggaactcagccgtttcgccatcaactctggacagaaaggctcgctgggcttttccgactgtacgctggaggcgatgcgcgcgctggcccgctacagcctgcagaacgacatccagacgctggtgacggtaaccaccgtaggcgtggagaagatgatgatccgtgccggcctggacgtatcgcgcttcggtccgcacctgaagatcggcatcgagcgcgcggtggccttgcgcatcgaactcaatgccaagacccagatcgcgctttacgggggagtgctggtggaacagcgactggcggtttca

SEQ ID NO: 12 - DNA, 3075 bp, synthetic construct
  source 1..3075: mol_type = other DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 13 - DNA, 53 bp, synthetic construct
  source 1..53: mol_type = other DNA
aaatctatctcatttgctagttataaaattatgaaatttgcataaattcttca

SEQ ID NO: 14 - DNA, 726 bp, synthetic construct
  source 1..726: mol_type = other DNA
atggccttggttgacggttttcttgagctggaacgctcaagtggaaaattggagtggagcgccatcctgcagaagatggcgagcgaccttggattctcgaagatcctgttcggcctgttgcctaaggacagccaggactacgagaacgccttcatcgtcggcaactacccggccgcctggcgcgagcattacgaccgggctggctacgcgcgggtcgacccgacggtcagtcactgtacccagagcgtactgccgattttctgggaaccgtccatctaccagacgcgaaagcagcacgagttcttcgaggaagcctcggccgccggcctggtgtatgggctgaccatgccgctgcatggtgctcgcggcgaactcggcgcgctgagcctcagcgtggaagcggaaaaccgggccgaggccaaccgtttcatggagtcggtcctgccgaccctgtggatgctcaaggactacgcactgcagagcggtgccggactggccttcgaacatccggtcagcaaaccggtggttctgaccagccgggagaaggaagtgttgcagtggtgcgccatcggcaagaccagttgggagatatcggttatctgcaactgctcggaagccaatgtgaacttccatatgggaaatattcggcggaagttcggtgtgacctcccgccgcgtagcggccattatggccgttaatttgggtcttattactctctgataataa

SEQ ID NO: 15 - DNA, 78 bp, synthetic construct
  source 1..78: mol_type = other DNA
ctcgagaaatcataaaaaatttatttgctttgttagcggagaagaattataatagattcaattgtgagcggataacaa

SEQ ID NO: 16 - DNA, 678 bp, synthetic construct
  source 1..678: mol_type = other DNA
atggatagcactgagaacgtcatcaagcccttcatgcgcttcaaggtgcacatggagggctccgtgaacggccacgagttcgagatcgagggcgagggcgagggcaagccttacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtga

SEQ ID NO: 17 - DNA, 42 bp, synthetic construct
  source 1..42: mol_type = other DNA
gcagcgaacgacgaaaattacgcccttgcagcgtgataataa

SEQ ID NO: 18 - DNA, 540 bp, synthetic construct
  source 1..540: mol_type = other DNA
atgaaaaaaactgctatcgctatcgctgttgctctggctggtttcgctactgttgctcaggcggcggccatggctgcacccgcccgctcgcccagccccagcacacagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaaacagcactgccctccaaccccggaaacttcctgtgcaacccagattatcacctttgaaagtttcaaagagaacctgaaggactttctgcttgtcatcccctttgactgctgggagccagtccaggaggcggccgctggatccgaacaaaagctgatctcagaagaagacctaaaggcctcgggggccgatcaccatcatcaccatcattga

SEQ ID NO: 19 - DNA, 486 bp, synthetic construct
  source 1..486: mol_type = other DNA
atgaacatgaaaaaactggctaccctggtttccgctgttgcgctaagcgccaccgttagtgcgaatgcgatggcaaaagacaccatcgcggccgcagaggcagatgatattttcggtgagctaagctctggtaagaatgcaccgaaaacggggggaggggcgaaagggaacaatgcttcgcctgccgggagtggtaatactaaaaacaatggcgcatcaggggccgatatcaataactatgccgggcagattaaatctgctatcgaaagtaagttctatgacgcatcgtcctatgcaggcaaaacctgtacgctgcgcataaaactggcacccgatggtatgttactggatatcaaacctgaaggtggcgatcccgcactttgtcaggctgcgttggcagcagctaaacttgcgaagatcccgaaaccaccaagccaggcagtatatgaagtgttcaaaaacgcgccattggacttcaaaccgtaa

SEQ ID NO: 20 - DNA, 4358 bp, synthetic construct
  source 1..4358: mol_type = other DNA
atgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagcc

SEQ ID NO: 21 - DNA, 4358 bp, synthetic construct; identical to SEQ ID NO: 20
  source 1..4358: mol_type = other DNA

SEQ ID NO: 22 - DNA, 4122 bp, synthetic construct
  source 1..4122: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggcatgcgaattcttatttgtagagctcggttaatttctcctcttgaattcactggccgtcgttttacaggatcttgtacagggcccttagaattccaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 23 - DNA, 21 bp, synthetic construct
  source 1..21: mol_type = other DNA
aagcttatcgatgataagctg

SEQ ID NO: 24 - DNA, 21 bp, synthetic construct
  source 1..21: mol_type = other DNA
taatgcggtagtttatcacag

SEQ ID NO: 25 - DNA, 39 bp, synthetic construct
  source 1..39: mol_type = other DNA
cttatcatcgataagcttttaaaccgcctgttttaaaac

SEQ ID NO: 26 - DNA, 38 bp, synthetic construct
  source 1..38: mol_type = other DNA
cctttgctcatatgtatatctccttcttaaagttaaac

SEQ ID NO: 27 - DNA, 31 bp, synthetic construct
  source 1..31: mol_type = other DNA
tatacatatgagcaaaggagaagaacttttc

SEQ ID NO: 28 - DNA, 37 bp, synthetic construct
  source 1..37: mol_type = other DNA
tgataaactaccgcattattattatcacgctgcaagg

SEQ ID NO: 29 - DNA, 30 bp, synthetic construct
  source 1..30: mol_type = other DNA
ttcattatccatcctccatcgccacgatag

SEQ ID NO: 30 - DNA, 19 bp, synthetic construct
  source 1..19: mol_type = other DNA
tacctggtggcattggctg

SEQ ID NO: 31 - DNA, 39 bp, synthetic construct
  source 1..39: mol_type = other DNA
ccaatgccaccaggtactcaagatcacgaatattcatgg

SEQ ID NO: 32 - DNA, 35 bp, synthetic construct
  source 1..35: mol_type = other DNA
ggaggatggataatgaacacagctaacaccacgtc

SEQ ID NO: 33 - DNA, 38 bp, synthetic construct
  source 1..38: mol_type = other DNA
taatgcggtagtttatcacagttaaattgctaacgcag

SEQ ID NO: 34 - DNA, 34 bp, synthetic construct
  source 1..34: mol_type = other DNA
ggtacctttctcctctttaatgaattcgtgtgag

SEQ ID NO: 35 - DNA, 23 bp, synthetic construct
  source 1..23: mol_type = other DNA
gcagcgaacgacgaaaattacgc

SEQ ID NO: 36 - DNA, 41 bp, synthetic construct
  source 1..41: mol_type = other DNA
gaggagaaaggtaccatgaccatgattacggattcactggc

SEQ ID NO: 37 - DNA, 48 bp, synthetic construct
  source 1..48: mol_type = other DNA
gataaactaccgcattatttttgacaccagaccaactggtaatggtag

SEQ ID NO: 38 - DNA, 46 bp, synthetic construct
  source 1..46: mol_type = other DNA
ttcgtcgttcgctgctttttgacaccagaccaactggtaatggtag

SEQ ID NO: 39 - DNA, 39 bp, synthetic construct
  source 1..39: mol_type = other DNA
gaggagaaaggtaccatgatcgtacaaattggtcggcgc

SEQ ID NO: 40 - DNA, 35 bp, synthetic construct
  source 1..35: mol_type = other DNA
gataaactaccgcattatgaaaccgccagtcgctg

SEQ ID NO: 41 - DNA, 33 bp, synthetic construct
  source 1..33: mol_type = other DNA
ttcgtcgttcgctgctgaaaccgccagtcgctg

SEQ ID NO: 42 - DNA, 52 bp, synthetic construct
  source 1..52: mol_type = other DNA
aatattgaaaaaggaagagtatgaataaggcctacagtatcatttggagcca

SEQ ID NO: 43 - DNA, 55 bp, synthetic construct
  source 1..55: mol_type = other DNA
tttgacagcttatcatcgatgctagctcagaagctgtattttatccccagtgctc

SEQ ID NO: 44 - DNA, 35 bp, synthetic construct
  source 1..35: mol_type = other DNA
atcgatgataagctgtcaaacatgagaattacaac

SEQ ID NO: 45 - DNA, 45 bp, synthetic construct
  source 1..45: mol_type = other DNA
actcttcctttttcaatattattgaagcatttatcagggttattg

SEQ ID NO: 46 - DNA, 23 bp, synthetic construct
  source 1..23: mol_type = other DNA
tgcatttgcagtcgacatgcacg

SEQ ID NO: 47 - DNA, 28 bp, synthetic construct
  source 1..28: mol_type = other DNA
cattgttattcacggtaccatgatggtg

SEQ ID NO: 48 - DNA, 32 bp, synthetic construct
  source 1..32: mol_type = other DNA
tgcaaatgcatttccgattgtggaaacaaccg

SEQ ID NO: 49 - DNA, 39 bp, synthetic construct
  source 1..39: mol_type = other DNA
ggtaccgtgaataacaatggaagcattgtcattaataac

SEQ ID NO: 50 - DNA, 19 bp, synthetic construct
  source 1..19: mol_type = other DNA
ggtagcggcagccaccatc

SEQ ID NO: 51 - DNA, 39 bp, synthetic construct; identical to SEQ ID NO: 49
  source 1..39: mol_type = other DNA

SEQ ID NO: 52 - DNA, 23 bp, synthetic construct
  source 1..23: mol_type = other DNA
gaattcgcatgccaccatcaaac

SEQ ID NO: 53 - DNA, 19 bp, synthetic construct
  source 1..19: mol_type = other DNA
cttcacttcctccaaatag

SEQ ID NO: 54 - DNA, 40 bp, synthetic construct
  source 1..40: mol_type = other DNA
tgcatactagtatgagcaaaggagaagaacttttcactgg

SEQ ID NO: 55 - DNA, 41 bp, synthetic construct
  source 1..41: mol_type = other DNA
ttcttagagctcggatccatccatgccatgtgtaatcccag

SEQ ID NO: 56 - DNA, 37 bp, synthetic construct
  source 1..37: mol_type = other DNA
atttggaggaagtgaagatgaaaaaaactgctatcgc

SEQ ID NO: 57 - DNA, 36 bp, synthetic construct
  source 1..36: mol_type = other DNA
ggtggcatgcgaattcttacggtttgaagtccaatg

SEQ ID NO: 58 - DNA, 35 bp, synthetic construct
  source 1..35: mol_type = other DNA
agcatactagtatgaccatgattacggattcactg

SEQ ID NO: 59 - DNA, 35 bp, synthetic construct
  source 1..35: mol_type = other DNA
tcttaggatccttatttttgacaccagaccaactg

(19) United States
(12) Patent Application Publication
(10) Pub. No.: US 2023/0038480 A1
(43) Pub. Date: Feb. 9, 2023

(54) SYSTEM FOR PRODUCITON OF HIGH YIELD OF RECOMBINANT PROTEINS

(71) Applicants: UNIVERSITY OF MARYLAND, COLLEGE PARK, College Park, MD (US); THE UNITED STATES OF AMERICA AS REPRESENTED BY THE SECRETARY OF THE ARMY, Washington, DC (US)

(72) Inventors: Kristina STEPHENS, Takoma Park, MD (US); William E. BENTLEY, Annapolis, MD (US); Jessica Lynn LIBA, Drexel Hill, PA (US); Ryan MCKAY, Peekskill, NY (US); Micaela EVERITT, Washington, DC (US)

(73) Assignees: UNIVERSITY OF MARYLAND, COLLEGE PARK, College Park, MD (US); THE UNITED STATES OF AMERICA AS REPRESENTED BY THE SECRETARY OF THE ARMY, Washington, DC (US)

(21) Appl. No.: 17/861,150

(22) Filed: Jul. 8, 2022

Related U.S. Application Data
(60) Provisional application No. 63/219,775, filed on Jul. 8, 2021.

(51) Int. Cl. C12P 21/02; C12N 5/10; A61K 38/19; C12N 1/20

(52) U.S. Cl. CPC C12P 21/02; C12N 5/10; A61K 38/19; C12N 1/20; C12N 2523/00; C12N 2510/00; C12N 2533/10; C12N 2500/02; C12N 2529/00

(57) ABSTRACT

The presently disclosure relates to a system and method for bioelectronic communications. In certain embodiments the system comprises a bacterial cell or cells that comprise a genetic system for high-efficiency over-expression and secretion of recombinant proteins in bacteria. In certain embodiments, the system and method operate in a “pump-then-burst release” fashion to rapidly achieve high yields extracellularly. In certain embodiments, the system and method include quorum sensing-derived regulation, which may enable auto-induction of a protein's expression and secretion.

### CROSS-REFERENCE TO RELATED APPLICATIONS

This application claims priority to U.S. Provisional Application No. 63/219,775 filed on Jul. 8, 2021, which is incorporated by reference in its entirety.

### STATEMENT REGARDING FEDERALLY SPONSORED RESEARCH

This invention was made jointly with the Army Research Laboratory and with government support under R21EB024102 awarded by the National Institutes of Health (NIH) and under CBET1805274 and ECCS1807604 awarded by the National Science Foundation (NSF). The government has certain rights in the invention.

### FIELD

The present disclosure generally relates to a cell culturing system, and specifically to an inducible, non-pathogenic bacterial cell culturing system that supports high overexpression followed by release of recombinant protein or signaling molecules. In some embodiments, the recombinant protein and signaling molecules are induced by the presence of electric charge and/or stimulate measurable voltage potential proportionate to the amount of molecule release in the system.

### BACKGROUND

Biological processes have been successfully explored using the ionic signaling modality to link with electronics; more recent strategies, including engineered optogenetics, innately-equipped electrogenic microbes, and/or recombinant metabolic components have enabled biological access to optical and electronic signaling³⁻⁷. As a more widely accessible alternative, redox-active molecules have recently been introduced as a bioelectronic signaling medium^{8, 9}. Redox-active molecules are well suited for bioelectronic communication by serving dually as electron carriers and biomolecular species, where electron flow is coupled to interconversion between biologically-distinct redox states¹⁰. Routine electrochemical instrumentation provides a programmable electronic interface, where redox events are directed in real-time by voltage parameters and use minimal infrastructure for versatile, miniaturizable, and in situ implementation¹¹. This provides an expedient tool to electronically access a wide repertoire of biomolecules and redox-sensitive proteins, thus potentially controlling biorecognition and associated genetic machinery^{12, 13}.

Tschirhart et al. have previously realized the concept of ‘electrogenetics’¹⁴, where electronic input was relayed using a redox mediator to dynamically activate gene expression in E. coli¹⁵. Until this disclosure, it was not known whether or how to produce electrode-generated redox signals without redox mediators but with surface-engineered bacteria.

### SUMMARY

The present disclosure relates to a genetic system for high-efficiency over-expression and secretion of recombinant proteins in E. coli that uniquely occurs in a “burst release” fashion. Related work in microbial secretion systems have been previously established as simple methodologies for protein recovery during its production, without need for cell lysis and separation steps. Additionally secretion is a route for expression of proteins that are otherwise cytotoxic, prone to aggregation, or difficult to express. Though genetic engineering strategies exist for protein secretion in E. coli, there are few choices: typically, heterologous proteins are fused to a periplasmic (secA) or extracellular secretion tag (YebF, OsmY), an outer membrane protein (OmpA), or involve other permeabilization-inducing mechanisms (hemolysin, curli system, kil switch). Limitations of these mechanisms include low protein yields, limitations to protein size, cytotoxic side effects, and complications in optimally regulating the required secretion machinery. Our system is simple, modular, and utilizes a fundamentally different mechanism than has been previously reported.

The present disclosure relates to a method of producing a recombinant amino acid in one or a plurality of bacterial cells in a culture vessel comprising a solid substrate, said method comprising: (a) contacting a first bacterial cell or a first population of isolated bacterial cells with the solid substrate, said substrate comprising at least one exterior surface, at least one interior surface and at least one interior chamber defined by the interior surface and at least one opening; (b) applying a cell medium into the culture vessel with a volume of cell medium sufficient to cover the at least one interior chamber; and (c) exposing the first bacterial cells or first population with an inducer for a time period sufficient to stimulate expression of the recombinant amino acid; wherein the first cell or first population of cells comprises a nucleic acid molecule comprising an expressible nucleic acid sequence encoding the amino acid operably linked to a regulatory sequence specific for association with the inducer.

In some embodiments, step (a) is preceded by transforming the first bacterial cell or first population of bacterial cells with the nucleic acid sequence. In some embodiments, the solid substrate comprises a base with a predetermined shape that defines the shape of the exterior and interior surface.

In some embodiments, the solid substrate comprises one or a combination of silica, plastic, ceramic, or metal and wherein the base is in a shape of a cylinder or rectangular prism or in a shape substantially similar to a cylinder or a rectangular prism, such that coat the interior surface of the base and define a cylindrical or substantially cylindrical interior chamber or compartment; and wherein the opening is positioned at one end of the cylinder.

In some embodiments, the inducer is peroxide, hydrogen peroxide, an oxidized form of OxyR or autoinducer 1.

In some embodiments, the nucleic acid molecule is free of secA, an extracellular secretion tag, or an outer membrane protein (e.g. OmpA or OmpB).

In some embodiments, the culture vessel further comprises a second population of bacterial cells; or a second and third population of cells.

In some embodiments, the first bacterial cell or the first population of cells comprises a second expressible nucleic acid sequence encoding OxyR, wherein the nucleic acid sequence encoding oxyR operably linked to a PoxyS promoter sequence.

In some embodiments, upon exposure to an inducer, the first bacterial cell or first population of bacterial cells stimulates expression of a cytokine.

In some embodiments, the solid substrate comprises at least one electrode or is positioned within about 10 millimeters from the solid substrate, and the method further comprises a step of exposing the electrode to voltages from about −0.1 to about −1.5 V.

In some embodiments, the method is performed without exposure to reduction-oxidation mediators.

In some embodiments, the solid substrate comprises a first, second and third vessel, each vessel of a size and shape sufficient to allow diffusion of protein, nutrients, and oxygen through the solid substrate in the presence of the cell culture medium; and wherein the first, second and third vessel each comprise a first, second, and third population of bacterial cells, respectively.

In some embodiments, the first, second and third vessels are in fluid communication.

In some embodiments the method further comprises the step of exposing the culture vessel to 37° Celsius and a level of carbon dioxide of no more than about 5.0% for a time sufficient to allow expression of the amino acid in the first cell or first population of cells in the interior chamber. In some embodiments, the first bacterial cell or first population of bacterial cells are a non-pathogenic strain of bacteria from a genus of Escherichia.

The present disclosure also relates to a system comprising: (i) a solid substrate comprising at least a first vessel, wherein the first vessel comprises a first bacterial cell or a first population of bacterial cells; (ii) an electrode positioned on or proximate to the solid substrate; (iii) a cell culture medium; wherein the solid substrate comprises at least one electrode or is positioned within about 10 millimeters from the first vessel; and wherein the first bacterial cell or population comprises at least a first and a second nucleic acid sequence, the first nucleic acid sequence comprising at least one non-constitutive promoter operably linked to the second nucleic acid sequence; the second nucleic acid encoding: (a) at least one therapeutic agent; or (b) a signaling molecule; wherein the non-constitutive promoter is an inducible promoter responsive to at least one stimuli, and the at least one stimuli comprises: (x) the presence of a certain density or a certain number of bacterial cells comprising the first and second nucleic acid sequences; or (y) the presence of an inducer.

In some embodiments, the system is in operable connection to at least one computer storage memory.

In some embodiments, the system of further comprises a digital display in operable connection to the at least one electrically conductive material by an electrical circuit capable of carrying an a electrical signal corresponding to a quantity of ion concentration in the first to the digital display, wherein the digital display is a configured to display concentration value of ion concentration and/or an amount of amino acid in a sample when the at least one electrically conductive material is in contact with the volume of the first vessel for a time period sufficient for an inducer to induce release of the amino acid sequence from the first bacterial cell or first bacterial population of cells.

In some embodiments, the first bacterial cell or first population of bacterial cells are non-pathogenic bacterial cells is chosen from one or a combination of the genera chosen from: Salmonella, Escherichia, Firmicutes, Bacteroidetes, Lactobacillus, Bifidobacteria, or Acidopholus.

In some embodiments, the bacterial cell comprises no more than five expressible exogenous nucleic acid sequences that are coding sequences, wherein the first exogenous nucleic acid sequence comprises at least one non-constitutive promoter operably linked to the second exogenous nucleic acid sequence that encodes the at least one therapeutic agent.

In some embodiments, the therapeutic agent is a cytokine. In some embodiments, the system further comprises a computer processor in operable connection with the at least one computer storage memory.

In some embodiments, the inducer is peroxide, hydrogen peroxide, autoinducer 1, or a derivative thereof. In some embodiments, the first bacterial cell or first population of bacterial cells are free of an exogenous nucleic acid sequence that encodes one or a combination of secA, an extracellular secretion tag, or an outer membrane protein (Omp).

In some embodiments, the system further comprises a second population of bacterial cells; or a second and third population of bacterial cells; each of the second and/or third population of bacterial cells comprising at least one nucleic acid molecule that comprises an expressible nucleic acid sequence encoding a signaling molecule operably linked to a regulatory sequence responsive to a recombinant product of at least one of the other bacterial cells or bacterial cell populations. In some embodiments, the first bacterial cell or the first population of cells comprises a second expressible nucleic acid sequence encoding OxyR and oxyS, wherein the nucleic acid sequence encoding oxyS operably linked to a PoxyS promoter sequence.

In some embodiments, the electrode is positioned within about 10 millimeters from the first bacterial cell or first bacterial cell population and wherein the system is free of a reduction-oxidation mediator.

In some embodiments, the first bacterial cell or first bacterial cell population is immobilized to the first vessel via an interaction between a protein on the bacterial cell surface and a metal coating on the surface of the first vessel.

In some embodiments, the signal-to-noise ratio of the system is from about 1.5 to about 2.3. In some embodiments, the signal-to-noise ratio is about 2.

The present disclosure also relates to a method of inducing electrostimulative release of a signaling molecule or therapeutic protein from a bacterial cell comprising: (a) growing one or more bacterial cells of a first population of cells in the disclosed system; (b) introducing one or more stimuli to the one or more bacterial cells.

In some embodiments, the method further comprises a step of measuring one or more responses from the one or more bacterial cells to the one or more stimuli based upon a product of an oxidation or reduction reaction conducted in the first vessel.

The present disclosure also relates to a method of selective secretion of signaling molecule or amino acid sequence in a first population of cells comprising: (a) growing one or more bacterial cells in culture; (b) introducing one or more stimuli to the one or more bacterial cells; wherein the one or more stimuli comprises exposing the one or more bacterial cells to an voltage potential or electrical current sufficient to cause selective secretion of the signaling molecule or amino acid sequence.

In some embodiments, the one or more bacterial cells are at least a portion of the first cell population in the system.

In some embodiments, the signaling molecule or amino acid sequence is a green fluorescent protein, a cytokine, AHL, beta-galactosidase, LacI gene product or DsRed.

In some embodiments, the method is free of a step of releasing the one or more bacterial cells through enzymatic lysis of the bacterial cell wall.

In some embodiments, the one or more bacterial cells have expressed but not secreted from about 1 to about 100 picograms of recombinant protein before step (b). In some embodiments, the one or more bacterial cells have expressed but not secreted from about 1 to about 100 nanograms of recombinant protein before step (b).

### BRIEF DESCRIPTION OF THE DRAWINGS

FIGS. 1A-1D depict the pump-then-burst genetic architecture. FIG. 1A depicts a map of plasmid pPTB used for pump-then-burst (PTB) secretion of a protein of interest, here DsRed-Express2 as a prototype. FIG. 1B depicts the architecture of PTB genetic circuit, with two divergent promoters: Pro_mT5, with a mutated LacO site for LacI-independent constitutive expression, regulates the gene of interest (GOI) to be secreted (eg DsRed-Express2) and Pro_LasI regulates a membrane pore (TolAIII) and co-translocated peptide by quorum sensing (*N-3-oxo-dodecanoyl homoserine lactone signal molecule) induction via LasR activation. Alternatively, an enzymatic reporter can be substituted to test promoter activity. FIG. 1C depicts the characterization of Pro_LasI quorum sensing induction, shown as a dose-response curve for enzymatic reporter activity. FIG. 1D depicts Pro_mT5 constitutive expression level optimized by varying the mutations of LacO sites (out of two sites embedded within the promoter). The relative constitutive red fluorescence is observably very strong, with a “medium” expression level being sufficient.

FIGS. 2A-2F depict time-dependent protein expression and extracellular secretion. FIG. 2A depicts a stained SDS-PAGE gel of concentrated conditioned media from induced culture timepoints. Secreted protein is DsRed-Express2, a 32 kDa red fluorescent protein and accumulates over 24 h. FIG. 2B depicts capillary gel electrophoresis of DsRed-Express2 in conditioned media from an uninduced culture. FIG. 2C depicts capillary gel electrophoresis of DsRed-Express2 in conditioned media from an induced culture. FIGS. 2B-C show time-intensifying bands at 32 kDa, like in FIG. 2A. FIG. 2D depicts optical density measurements at 600 nm for uninduced and induced cultures over time. FIG. 2E depicts the relative fluorescence measurements of uninduced and induced culture conditioned media over time. FIG. 2F depicts the quantified concentrations of secreted protein (DsRed-Express2) over time from uninduced and induced cultures.

FIGS. 3A-3B depict photographs of uninduced and induced cultures with secretable DsRed-Express2. FIG. 3A depicts culture tubes angled to show visible DsRed fluorescence in uninduced and induced cell pellets. FIG. 3B depicts culture tubes angled to show visible DsRed fluorescence in uninduced and induced supernatant media.

FIGS. 4A-4C depict quorum sensing in co-cultures for autoinduction of protein secretion. FIG. 4A depicts an illustration of a triple coculture containing a strain that sends quorum sensing signals mixed with cells that can be induced by the signal enables autoinduction. One cell type with pPTB is a “Secretor” that allows inducible secretion of DsRed-Express2. Another cell type with inducible enzyme activity is a “Reporter” and expresses non-secreted DsRed-Express2. FIG. 4B depicts fluorescence excitation of coculture supernatants and pellets having differing ratios of Secretor to Reporter. Because both Secretor and Reporter express DsRed, all pellets are fluorescent, but because only the Secretor allows extracellular release of DsRed, the supernatant fluorescence increases as the fraction of Secretor increases. FIG. 4C depicts the quantified fluorescence of the coculture supernatant and enzymatic activity levels (normalized to max values) match the relative ratios of Secretor to Reporter.

FIGS. 5A-5C depict a drawing of information flow through an electronically-interfaced biological network. FIG. 5A depicts the connectivity established between electronics and a microbial community using biomolecular redox events to transduce bioelectronic signaling by simultaneously mediating electron flow and eliciting biological interactions. The result is an electronically-controlled biological local area network (BioLAN) that propagates the signal across microbial subpopulations and onto to external environments for multiplexed actuation. One subpopulation transduces received information into electronic output that returns BioLAN system status and a second produces biological outputs. FIG. 5B depicts the transduction of an electronic-encoded input to a biologically-recognized signal, hydrogen peroxide, via electrochemically-controlled oxygen reduction. BioLAN cells assembled at the electrode (via gold-binding peptide surface display, GBP₃-AIDAc) intercept peroxide to activate electrogenetic expression of LasI. LasI then synthesizes acyl homoserine lactone (AHL) for signal routing. FIG. 5C depicts the indirect connection of additional cells in the BioLAN to electrogenetically-controlled events through AHL activation. Verifier cells detect the routed AHL signal via LasR and produce the β-galactosidase (β-gal) enzyme. β-gal cleaves PAPG to PAP, which is detected by electrochemical oxidation. This acts to transmit the propagated signal back to the electronics and thereby reports on BioLAN system connectivity. Additionally, Actuator cells respond to the AHL signal by upregulating TolAIII-mediated membrane porosity, which co-releases a therapeutically-relevant granulocyte macrophage colony stimulating factor (GMCSF) and overexpressed DsRedExpress II fluorescent protein out of the cell via diffusion.

FIGS. 6A-6F depict peroxide-driven electrogenetic control. FIG. 6A depicts OxyR-regulated gene expression: hydrogen peroxide oxidizes OxyR, resulting in disulfide bond formation. OxyR(o) activates expression of a gene of interest (goi) under the PoxyS promoter. FIG. 6B Dose-response of model-predicted intracellular OxyR(o) fraction at 10 min and saturation-normalized expression of reporter proteins (β-galactosidase, sfGFP) to hydrogen peroxide, measured by Miller Assay (at 60 min) and flow cytometry (at 45 min), respectively. Experimental data indicated by data points. Fitted models of relative protein levels shown as solid lines. Grid lines indicate induction threshold (25 μM peroxide) and corresponding OxyR(o) peak (fOxyR(o)=0.644) required to achieve saturated protein expression. FIG. 6C depicts a timecourse of model-predicted intracellular OxyR(o) fraction and data of saturation-normalized expression levels of reporter proteins (β-galactosidase, LasI, each ssrA-tagged) when induced by 25 μM peroxide, respectively. For FIGS. 6B-C, experimental data are indicated by data points and fitted models of protein levels are shown as solid lines. FIG. 6D depicts the schematic of a setup for electrochemical reduction of oxygen to hydrogen peroxide to drive OxyR(o)-mediated gene expression. FIG. 6E depicts the measured peroxide concentration in the working electrode solution over time, with a constant −0.4 V applied to electrodes of indicated surface areas. FIG. 6F depicts electrogenetic β-galactosidase expression over time in response to the indicated electrochemical pulse lengths −0.4 V. ‘0 s’ condition indicates basal β-galactosidase activity. For ‘+H2O2’ positive control dataset, 25 μM hydrogen peroxide was exogenously added to culture. Data in FIG. 6B and FIG. 6C represents means of biological triplicates and data in FIG. 6E represents means of technical triplicates. Error bars represent standard deviation.

FIGS. 7A-7D depict the contribution of electrochemical generation and bacterial consumption rates to hydrogen peroxide flux simulated at an electrode. FIG. 7A depicts a simulation of peroxide flux at a distance from the electrode interface over time, based on surface-area dependent rate of charge, average stoichiometric efficiency of oxygen reduction, and the presence of mobile peroxide-consuming cells in the electrode's vicinity. 0-300 s timepoints account for a biased electrode with continuous peroxide generation (“eGeneration”). For 360-600 s timepoints, the electrochemical reaction has been terminated. FIG. 7B depicts a simulation of peroxide flux at an electrode interface, differing from FIG. 7A due to the positioning of the cells at the electrode interface. FIG. 7C depicts the modeled ranges of peroxide depletion over time, predominantly by cell decomposition, based on peroxide electro-generation in the presence of mobile cells (tracked across distance intervals from electrode, Z1, Z2, and Z3), and at the electrode interface for immobilized cells. FIG. 7D depicts the projected ranges of OxyR oxidation state over time for cells corresponding to the conditions denoted in FIG. 7C. Oxidized (OxyR(o)) levels reported as fractions of total intracellular OxyR. Additionally, the population fraction of cells applicable to the OxyR models is reported, based on cell. For planktonic cells, the peroxide depletion rates in FIG. 7C and OxyR(o) fractions in FIG. 7D are denoted as a range of values within three zones proximal to the electrode interface: Z1 (0-0.225 mm), Z2 (0.225-0.625 mm), and Z3 (0.625-3.5 mm).

FIGS. 8A-8E depict the assembly of electrogenetic cells onto gold surfaces via peptide surface display. FIG. 8A depicts a homology model of the AIDA autotransporter structure with an N-terminally-linked fusion of ‘GBP₃⁺’ gold-binding peptide and hexameric histidine tag. FIG. 8B depicts a transmission electron micrograph of an E. coli cell lacking genes for GBP₃ surface display labeled with 20 nm gold nanoparticles. FIG. 8C depicts a transmission electron micrograph of a GBP₃⁺E. coli cell labeled with 20 nm gold nanoparticles. Scale bars=200 nm. FIG. 8D depicts the coverage of E. coli cells (−/+pGBP₃) immobilized to either a gold-coated (Au) or uncoated (SiO₂) silicon wafer and stained with Syto-9. FIG. 8E depicts a demonstration of electrogenetic activation of gene expression by applying a voltage to electrodes with immobilized cells hosting various reporters. Quantification for FIG. 8D and FIG. 8E are based on three biological replicates per condition and image analysis of technical replicates with standard deviation of data reported.

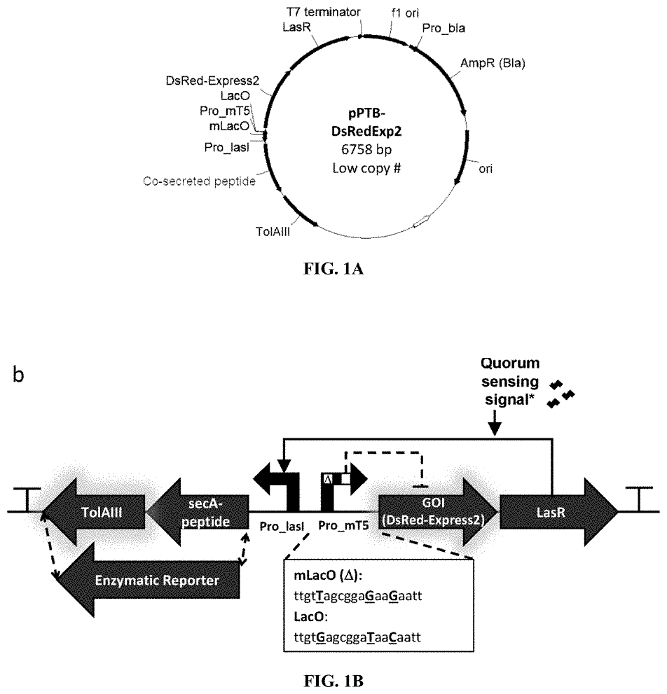

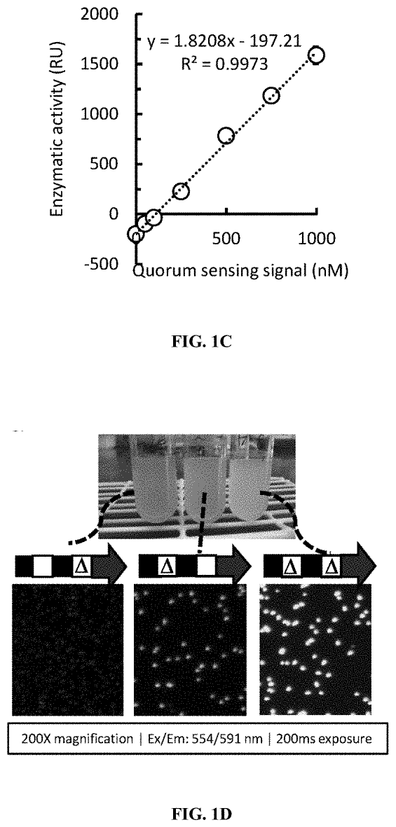

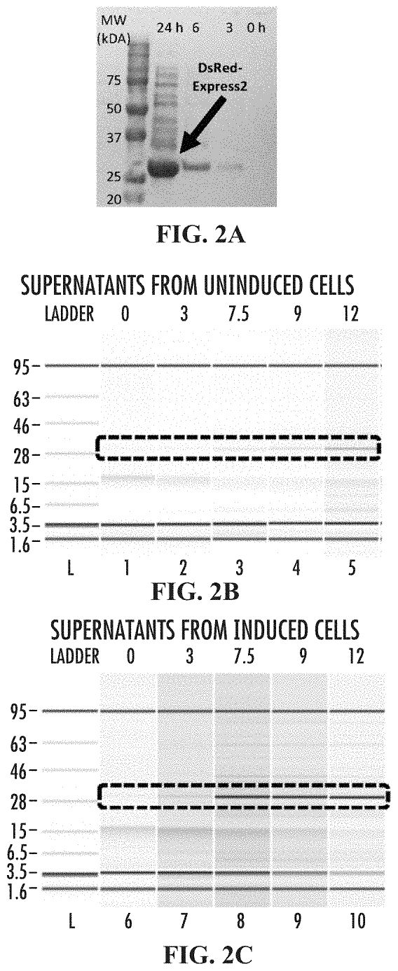

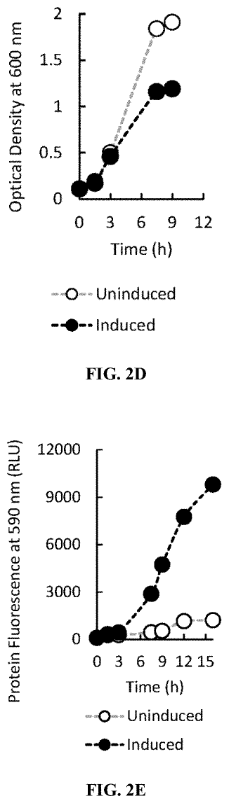

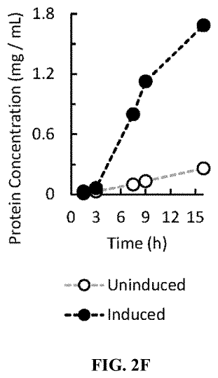

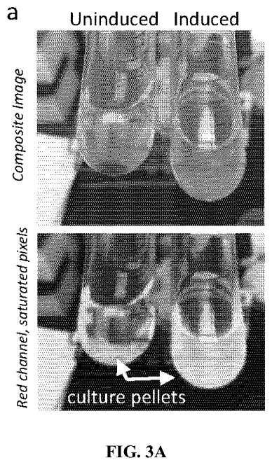

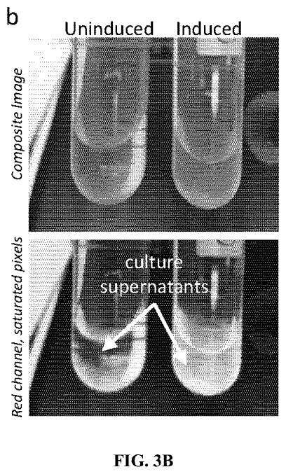

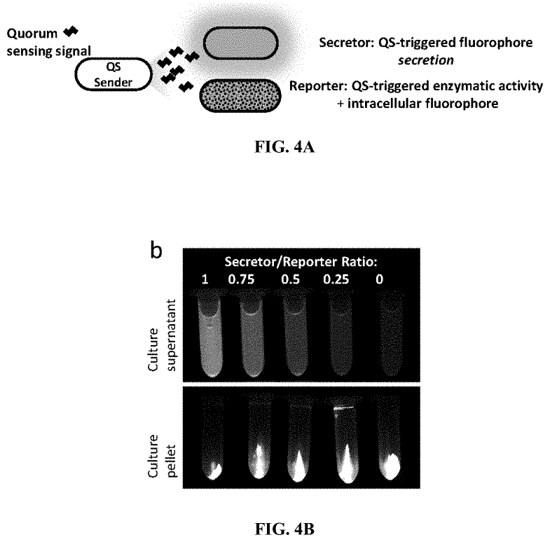

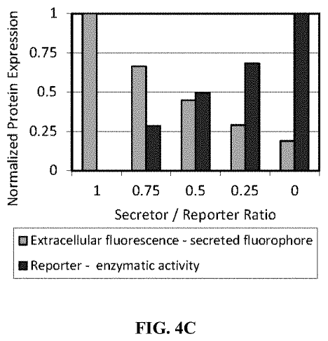

A. FIGS. 9A-9E depict electronic information flow through an engineered two-member community. FIG. 9A depicts a schematic of information flow from the electrode, through the Routing and Verification cells with AHL, and back out to the electrode. Redox signals provide transduction between cells and electrodes. FIG. 9B depicts electronic output, quantified by DPV peak current of PAP oxidation, from Verifier cells with or without AHL induction, measured over time. FIG. 9C depicts verifier cell output, measured both electronically (PAP oxidation current) and with the Miller Assay in response to induction with different charges. Routing cells are planktonic. FIG. 9D depicts indicators of the applied charge (i): AHL production levels (ii); b-gal activity (iii); and electronic output of a two-population bioelectronic system described in FIG. 9A either, with or without applied charge. FIG. 9E depicts the signal-to-noise ratios (On/Off) at either 1.5 or 3 hours post-induction. In FIG. 9D, means of biological triplicates are shown; error bars indicate standard deviation.

FIGS. 10A-10E depict BioLAN function. FIG. 10A depicts a schematic of information flow into the BioLAN, communication between members, and BioLAN output. FIG. 10B depicts normalized protein expression from BioLAN communities in which the indicated ratios of Verifier/Actuator cell ratios were used. FIG. 10C depicts measurements of applied charge dose (i), AHL production levels (ii), electronic output from Verifier cells (iii) and GMCSF and DsRed production by Actuator cells (iV) from BioLAN systems induced electronically (ON) or kept uninduced (OFF). Means and standard deviations of biological quadruplicates are shown. FIG. 10D depicts a schematic showing BioLAN connection to a computer and information flow between them. FIG. 10E depicts representative traces of real-time measurements of input charge (i) and output current (ii), which precedes the detection of Actuator cell production of DsRed and GMCSF (diamonds). Data is representative and corresponds to that in FIG. 10C.

FIGS. 11A-11C depict the evaluation of E. coli interactions with hydrogen peroxide. FIG. 11A depicts bacterial viability after peroxide dose exposure, measured for non-viable cells by counting a propidium iodide stained cell fraction and for viable cells by counting colony forming units. FIG. 11B depicts peroxide depletion data for cultures at varying cell densities (0.025, 0.1, and 0.4 OD600) shown as experimental measurements (circles) and modeled behavior (lines). FIG. 11C depicts the modeled extracellular peroxide concentration and resultant intracellular fraction of OxyR(o) over time for peroxide concentrations between 0 to 100 μM.

FIGS. 12A-12F depict modeled experimental data for hydrogen peroxide-regulated protein expression and activity. FIG. 12A depicts the timecourse of cell density and 25 μM-induced expression levels of a protein exhibiting either zero-order decay due to cell division or first-order decay influenced by degradation tag. FIG. 12B depicts a modeled OxyR-regulated protein expression timecourse across a range of 0 to 100 μM peroxide adjusted to experimental data for β-galactosidase. FIG. 12C depicts a modeled OxyR-regulated expression timecourse across a range of 0 to 100 μM peroxide adjusted to experimental data for sfGFP fluorescence. FIG. 12D depicts a modeled OxyR-regulated expression timecourse across a range of 0 to 100 μM peroxide adjusted to experimental data for the percentage of sfGFP-expressing cells in a population. FIG. 12E depicts modeled OxyR-regulated expression timecourses across a range of 0 to 100 μM peroxide adjusted to experimental data for LasI level and LasI-dependent acyl-homoserine lactone (AHL). FIG. 12F depicts standard curves for AHL concentrations between 0 and 400 nM, measured by bioluminescence assay. For plots in FIGS. 12B-12F, color-coded legends indicate peroxide condition for models (solid lines); square data points indicate experimental dose-response results and circles indicate experimental timecourse results. Data for FIG. 12E were quantified by bioluminescence assay, using Standard Curve #2 in FIG. 12F.

FIG. 13 depicts a series of fluorescence histograms for peroxide-induced sfGFP expression. Experimental flow cytometry data for pOxy-sfGFP cells after 45 min induction with hydrogen peroxide (0-100 μM). Population distribution model parameters (τ, α) were derived from the base 10 logarithm of the fluorescence threshold and standard deviation, given a normal distribution.

FIGS. 14A-B depict factors affecting electrogenetic protein expression. FIG. 14A depicts the influence of cell density on sfGFP fluorescence (as green population % exceeding fluorescence threshold and mean relative fluorescence of population) when induced with 100 μM peroxide. FIG. 14B depicts an expression timecourse when induced with 25 μM peroxide of sfGFP fluorescence (with ssRA degradation tag) and LasI-synthesized acyl-homoserine lactone (AHL, quantified by Curve 1 (FIG. 12F), compared between LasI with and without the ssRA tag inclusion.

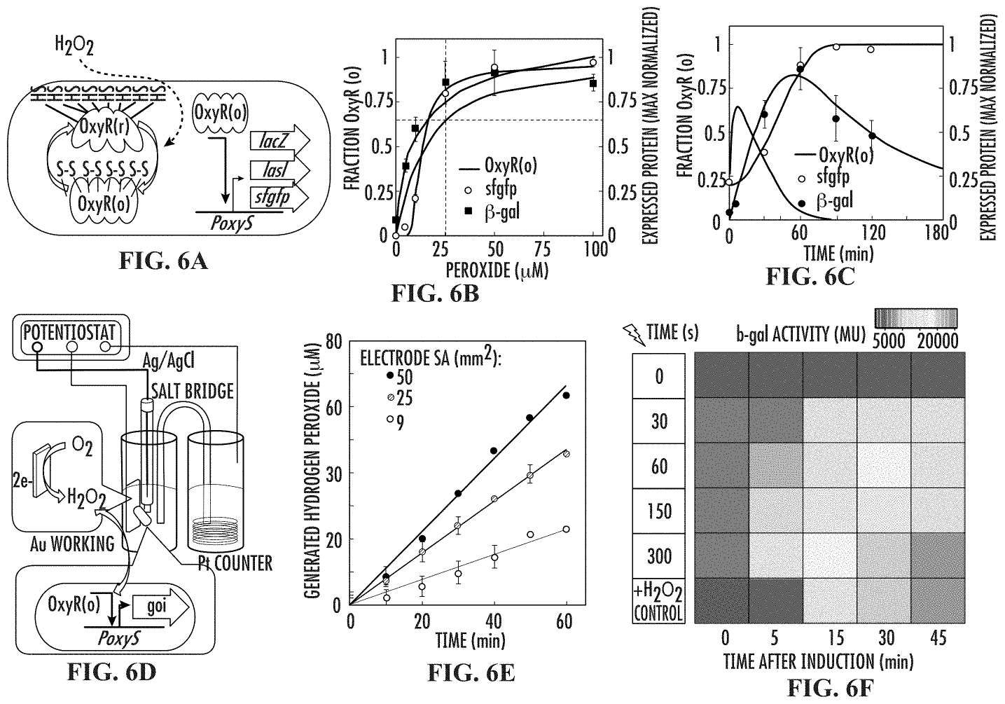

FIGS. 15A-15F depict the characterization of an electrochemical peroxide generation rate. FIG. 15A depicts the standard correlation between hydrogen peroxide concentration in M9 medium and spectrophotometric absorbance at 585 nm using a colorimetric peroxide assay. FIG. 15B depicts peroxide accumulation over time with constant voltage in various physiologically-compatible solutions: LB (lysogeny broth), M9 (minimal medium), PB (phosphate buffer). All set-ups used a split electrochemical configuration with conductivity across a salt bridge. PB was also tested with electrochemistry occurring in a single chamber (no salt bridge). FIG. 15C depicts the charge transfer during electrochemical peroxide generation using electrodes of indicated surface area. Inset shows picture of representative wells of a hydrogen peroxide assay during the timecourse of peroxide generation from indicated electrodes. Data represents the means of technical triplicates and error bars their standard deviation. FIG. 15D depicts a correlation of applied charge to accumulated peroxide molecules based on data in FIG. 15C and FIG. 6E. FIG. 15E depicts differences in rates of charge transfer and corresponding peroxide accumulation as a function of aeration (stirring or no stirring). FIG. 15F depicts the experimental stoichiometric efficiency of oxygen reduction to yield hydrogen peroxide for various electrode dimensions and stirring conditions. Standard deviation of mean efficiency (0.58) shown by error bars.

FIGS. 16A-16C depict electrogenetic induction and effects on cell growth. FIG. 16A depicts sfGFP fluorescence, measured by flow cytometry, of cells induced with the indicated peroxide-generating charges after 45 minutes. FIG. 16B depicts lag time growth effect of bacterial cultures with either exogenously-added or electrochemically-generated peroxide. FIG. 16C depicts and the doubling time growth effect with either exogenously-added or electrochemically-generated peroxide. Bar graphs depict the means of technical triplicates and error bars represent their standard deviations.

FIG. 17 depicts the simulated electro-generated peroxide levels at (solid lines) and after (dotted lines) specified duration, in systems without the presence of cells.

FIGS. 18A-18B depict a comparison of peroxide uptake between peroxide-induced planktonic cells and electro-induced electrode-immobilized cells. FIG. 18A depicts per-cell peroxide uptake rate for cells at 0.025 OD induced with 25 μM peroxide or cells-on-chip electro-induced for 300 s. FIG. 18B depicts the cumulative amounts of peroxide consumed for cells at 0.025 OD induced with 25 μM peroxide or cells-on-chip electro-induced for 300 s.

FIGS. 19A-19C depict metal nanoparticle binding to cell surfaces with surface display of GBP₃-His₆ peptide. FIG. 19A depicts corresponding fluoresence and phase contrast images of E. coli cells and CdSe/ZnS quantum dots (QD, emission=520 nm). FIG. 19B depicts the forward/side scatter distributions of QD-labeled E. coli with the fluorescent-gated population represented within the outlined data points. FIG. 19C depicts transmission electron microscopy images of E. coli with 20 nm gold nanoparticles. In FIGS. 19A-19C, top row shows GBP₃⁺ cells and bottom row shows GBP₃+ cells.

FIGS. 20A-20B depict characteristics of gold-immobilized cells. FIG. 20A depicts measured peroxide depletion by electrode-immobilized or planktonic cells, with concentration over time subtracted from the starting level, 100 μM. Bottom plot shows the consumption rate of each normalized to cell number, determined by starting optical density for planktonic cells or image analysis of fluorescently stained immobilized cells. All data for gold-immobilized samples is an average of four biological replicates with standard deviation indicated by error bars. FIG. 20B depicts the observed cell viability (% dead) of electrode-immobilized cells after exposure to varying electro-inducing charges, based on image analysis of live/dead fluorescence staining on-chip done by ImageJ. Means of at least biological triplicates and the corresponding standard deviations are shown. Bottom pictures show fluorescence images of PI-stained GBP₃-immobilized cells after electro-induction with the indicated charge or no induction.

FIGS. 21A-21B depict the characterization of LacZ reporter cells in response to acyl homoserine lactone (AHL) induction. In FIG. 21A, measurement of β-galactosidase activity (in Miller Units) for pAHL-LacZ+ cells is depicted as a response to an AHL dose up to 1.1 μM induction at 90 min. In FIG. 21B, β-galactosidase activity measurements are depicted as a timecourse without induction (0 μM) or with 1.1 μM AHL introduced at 0 min. All data represent the averages of technical triplicates, with standard deviation reported.

FIGS. 22A-22B depict the bioelectronic interface. FIG. 22A depicts the average surface area of working electrodes shown in the experiment associated with FIG. 9D. FIG. 22B depicts the cell coverage for this experiment.

FIGS. 23A-23C depict electronic information flow through a single bi-directionally-connected cell type. FIG. 23A depicts a schematic of information flow from the electrode, through the cell (peroxide induction of LacZ, and enzymatic PAP generation) and back out to the electrode, where PAP oxidation provides an electronic output. FIG. 23B depicts electronic output, quantified by DPV peak current of PAP oxidation, from cells with or without exogenous peroxide induction, measured over time. FIG. 23C depicts control-normalized charge input (peroxide formation) and cell electronic output (PAP oxidation peak current), from electrode-immobilized cells. Systems were set up with differing electrode surface areas, as indicated, with other system parameters remaining the same.

FIG. 24 depicts timecourse levels of extracellular GMCSF and DsRed secreted by Actuator cells either uninduced (−) or induced with 1.1 μM acyl homoserine lactone (+).

FIG. 25 depicts a correlation between p-aminophenol concentration and electrochemical measurements (peak height of differential pulse voltammogram (DPV).

### DETAILED DESCRIPTION

Some embodiments provide compositions comprising at least one bacterial cell that comprises one or more exogenous plasmids, wherein at least one plasmid comprises a heterologous nucleic acid sequence that encodes a therapeutic agent operably linked to an non-constitutive and/or inducible promoter; such non-constitutive and/or inducible promoter modulates the expression of the nucleic acid encoding the therapeutic agent in the presence of at least one stimulus. In some embodiments, the inducible and non-constitutive promoter only induces the expression of the nucleic acid encoding the therapeutic agent in the presence of a certain density or number of bacterial cells comprising the inducible promoter and/or the nucleic acid that encodes the therapeutic agent. When such a bacterial cell reproduces in vivo (either within the disclosed systems or administered without the system), such as when the bacterial cell is implanted or in the gut of a mammal, the clonally derived progeny of the at least one bacterial cells may reach a certain density or number that such density or number triggers the expression of the therapeutic molecule or signaling molecule. The ability of a bacterial cell to modulate expression of a gene in the presence of a certain number or density of cells is quorum sensing. In some embodiments, the non-constitutive or inducible promoter has only a single stimulus that is a certain number or density of cells. In some embodiments, the non-constitutive and/or inducible promoter responds to at least one, two, three, four, five or more stimuli but none of the stimuli require administration or exposure of the stimulus or stimuli external to the subject. In some embodiments, the stimulus or stimuli necessary to induce expression of the nucleic acid encoding the therapeutic molecule or signaling molecule requires exposure to or administration of a voltage or current before, simultaneously to, or after administration of the at least one bacterial cell.

Specific preferred embodiments of the present invention have been described here in sufficient detail to enable those skilled in the art to practice the full scope of invention. However it is to be understood that many possible variations of the present invention, which have not been specifically described, still fall within the scope of the present invention and the appended claims. Hence these descriptions given herein are added only by way of example and are not intended to limit, in any way, the scope of this invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of “Consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively.

The term “more than 2” as used herein is defined as any whole integer greater than the number two, e.g. 3, 4, or 5.

The term “plurality” as used herein is defined as any amount or number greater or more than 1.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

As used herein, the terms “administering” and “administration” refer to any method of providing a disclosed signaling molecule or therapeutic molecule to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent based upon exposure of the device or composition to an inducer and the location of where the device may be localized within the subject. In various embodiments, a preparation can be administered therapeutically; that is, administered to treat an existing disease or condition, such as a gastrointestinal disorder. In further various embodiments, a preparation can be administered prophylactically; that is, administered for prevention of a disease or condition.

The terms “amino acid” refer to a molecule containing both an amino group and a carboxyl group bound to a carbon which is designated the a-carbon. Suitable amino acids include, without limitation, both the D- and L-isomers of the naturally-occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs including non-natural analogs.

The term “bioreactor” refers to an enclosure or partial enclosure in which cells are cultured, optionally in suspension. In some embodiments, the bioreactor refers to an enclosure or partial enclosure in which cells are cultured where said cells may be in liquid suspension, or alternatively may be growing in contact with, on, or within another non-liquid substrate including but not limited to a solid growth support material. In some embodiments, the solid growth support material, or solid substrate, comprises at least one or a combination of: silica, plastic, metal, hydrocarbon, or gel. The disclosure relates to a system comprising a bioreactor comprising one or a plurality of culture vessels into which neuronal cells may be cultured in the presence or cellular growth media.

The terms “cell culture medium” refer to a liquid or gel designed to support the growth of microorganisms, cells. In some embodiments, the cells are prokaryotic cells or populations of cells disclosed herein.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a cell (e.g., a bacterial cell). In some embodiments contacting includes contacting a bacterial cell to a solid substrate.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores or vessels or indentations that allow diffusion of protein, nucleic acid, nutrients (such as heavy metals and hormones) antibiotics, and/or other cell culture medium components through the pores. In some embodiments, the vessels are not more than about 0.1, 0.5 1.0, 2, 3, 4, 5, 6, 7, 8, 9, 10, 15, 20, 25, 30, 40, 50 millimeters wide. In some embodiments, the culture vessel comprises a polymer or coating on a first portion of the vessel (typically a defined bottom) that facilitates binding with cellular proteins or extracellular proteins. In some embodiments, each vessel or pore or compartment within which a disclosed cell or population of cells is positioned is free of a redox mediator. In some embodiments, the culture vessel is designed to contain a hydrogel or hydrogel matrix and various culture mediums. In some embodiments, the culture vessel consists of or consists essentially of a plastic with a layer or coating of metal, such as gold. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated bacterial cells.

Disease, disorder, and condition are used interchangeably herein. “Disease” or “condition” refer to a state of being or health status of a patient or subject capable of being treated with a compound, pharmaceutical composition, or method provided herein. In some embodiments, the disease is a disease related to (e.g., characterized by) modulation of NO neuron activity. In some embodiments, the disease is a gastrointestinal intestinal disorder. Such disorder include gastritis, gastroenteritis, celiac disease, Crohn's disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome. In some embodiments, the disorder is chosen from those disclosed in Table Y.

The term “electrical stimulation” refers to a process in which the cells are being exposed to an electrical current of either alternating current (AC) or direct current (DC). The current may be introduced into the solid substrate or applied via the cell culture media or other suitable components of the cell culture system. In some embodiments, the electrical stimulation is provided to the disclosed device or system by positioning one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the cell culture system.

The terms “exogenous gene” or “exogenous nucleic acid” (interchangeable) means a nucleic acid that has been recombinantly introduced into a cell, which encodes the synthesis of RNA and/or protein. In some embodiments, the exogenous gene is introduced by transformation. In some embodiments, the exogenous gene is introduced into the cell by electroporation. A transformed cell may be referred to as a recombinant cell, into which additional exogenous gene(s) may be introduced. The exogenous gene put into the host species may be taken from a different species (this is called heterologous), or it may naturally occur within the same species (this is homologous as defined below). Therefore, exogenous genes subsume homologous genes that are integrated within or introduced to regions of the genome, episome, or plasmid that differ from the locations where the gene naturally occurs. Multiple copies of the exogenous gene may be introduced into the cell. An exogenous gene may be present in more than one copy within the host cell or transformed cell. In some embodiments, the microorganism comprises between and including 1 and 10,000 copies of the nucleic acid that encodes an exogenous protein. In some embodiments, the microorganism comprises between and including 1 and 1,000 copies of the nucleic acid that encodes an exogenous protein. In some embodiments, the microorganism comprises between and including 1 and 10,000 copies of the nucleic acid that encodes an exogenous protein. In some embodiments, the microorganism comprises between and including 1 and 1,000 copies of the nucleic acid that encodes an exogenous protein. In some embodiments, the microorganism comprises between and including from about 1 to about 500 copies of the nucleic acid that encodes an exogenous protein. In some embodiments, the exogenous gene is maintained by a cell as an insertion into the genome or as an episomal molecule. In some embodiments, the microorganism comprises no more than 1, 2, 3, 4, 5, 6, 7, 8, 9, 10, 20, 30, 40, 50, 60, 70, 80, 90, 100, or 1000 copies of the one or more nucleic acids that encode one or more exogenous proteins.

The terms “functional fragment” means any portion of a polypeptide or nucleic acid sequence from which the respective full-length polypeptide or nucleic acid relates that is of a sufficient length and has a sufficient structure to confer a biological affect that is similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence upon which the sequence is derived. The present invention also comprises functional fragments of nucleotide sequences that encode a polypeptide capable of enzymatic activity, substrate activity, polarization activity, toxin activity, or antidote to toxin activity as disclosed herein in an animal. In some embodiments, the functional fragment are DNA or amino acid fragments selected from at least one of the various encoding nucleotide sequences of the present disclosure, including SEQ ID NO: 1 through 18 and can be any of the following described DNA fragments, as it applies to the specific encoding nucleic acid sequence provided herein. In some embodiments, DNA fragments can comprise 30 or more, 45 or more, 60 or more, 75 or more, 90 or more, 120 or more, 150 or more, 180 or more, 210 or more, 240 or more, 270 or more, 300 or more, 360 or more, 420 or more, 480 or more, 540 or more, 600 or more, 660 or more, 720 or more, 780 or more, 840 or more, 900 or more, 960 or more, 1020 or more, 1080 or more, 1140 or more, 1200 or more, 1260 or more, 1320 or more, 1380 or more, 1440 or more, 1500 or more, 1560 or more, 1620 or more, 1680 or more, or 1740 or more, 1800 or more, 2000 or more, 2100 or more, 2200 or more, 2300 or more, 2400 or more, 2500 or more, 2600 or more, 2700 or more, 2800 or more, 2900 or more, 3000 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10000 or more, 10100 or more nucleotides. In some embodiments, DNA fragments can comprise coding sequences for accessory proteins such as known ligands to tumor associated antigens expressed on the surface of tumor cells. In some embodiments, DNA fragments can comprise fewer than 60, fewer than 75, fewer than 90, fewer than 120, fewer than 150, fewer than 180, fewer than 210, fewer than 240, fewer than 270, fewer than 300, fewer than 360, fewer than 420, fewer than 480, fewer than 540, fewer than 600, fewer than 660, fewer than 720, fewer than 780, fewer than 840, fewer than 900, fewer than 960, fewer than 1020, fewer than 1080, fewer than 1140, fewer than 1200, fewer than 1260, fewer than 1320, fewer than 1380, fewer than 1440, fewer than 1500, fewer than 1560, fewer than 1620, fewer than 1680, or fewer than 1740, fewer than 1800, fewer than 1900, fewer than 2000, fewer than 2100, fewer than 2200, fewer than 2300, fewer than 2400, fewer than 2500, fewer than 2600, fewer than 2700, fewer than 2800, fewer than 2900, fewer than 3000, fewer than 4000, fewer than 5000, fewer than 6000, fewer than 7000, fewer than 8000, fewer than 9000, or fewer than 10000 nucleotides. In some embodiments, the functional fragments are nucleic acid fragments of SEQ ID NO: 1 through 20 and include one or more nucleic acid derivatives. In some embodiments, the functional fragments are nucleic acid fragments of SEQ ID NO: 1 through 20 and include more than about 5, 10, 15, 20, 25, or 30 nucleic acid derivatives.

As used herein, the term “genetic construct” is meant to refer to the DNA or RNA molecules that comprise a nucleotide sequence that encodes protein. The coding sequence includes initiation and termination signals operably linked to regulatory elements including a promoter and polyadenylation signal capable of directing expression in the cells of the individual to whom the nucleic acid molecule is administered.

The term “host cell” as used herein is meant to refer to a cell that can be used to express a nucleic acid, e.g., a nucleic acid of the disclosure. The host cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. Suitable prokaryotic cells are provided in Table Z. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “hybridize” as used herein is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “isolated” as used herein means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptides or molecules of the disclosure.

The term “pharmaceutically acceptable” as used herein refers to approved or approvable by a regulatory agency of the Federal or a state government or listed in the U.S. Pharmacopeia or other generally recognized pharmacopeia for use in animals, including humans.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” of signaling molecules or therapeutic molecules as used herein may comprise an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is from about 0 to about 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

The term “plastic” refers to biocompatible polymers comprising hydrocarbons. In some embodiments, the plastic is selected from the group consisting of: Polystyrene (PS), Poly acrylo nitrile (PAN), Poly carbonate (PC), polyvinylpyrrolidone, polybutadiene (PVP), Polyvinyl butyral (PVB), Poly vinyl chloride (PVC), Poly vinyl methyl ether (PVME), poly lactic-co-glycolic acid (PLGA), poly(1-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any of the foregoing polymers.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide. That is, e.g., a purified polypeptide is a polypeptide that is at least from about 70 to about 100% pure, i.e., the polypeptide is present in a composition wherein the polypeptide constitutes from about 70 to about 100% by weight of the total composition. In some embodiments of the disclosed methods, the method comprise a final step of purifying in vitro produced therapeutic molecules or signaling such that the recombinant products may be purified and useful for GLP or therapeutic standards. In some embodiments, the purified polypeptide is from about 75% to about 99% by weight pure, from about 80% to about 99% by weight pure, from about 90 to about 99% by weight pure, or from about 95% to about 99% by weight pure.

The term “seeding” as used herein is defined as transferring an amount of cells into a new culture vessel. The amount may be defined and may use volume or number of cells as the basis of the defined amount. The cells may be part of a suspension.

The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores or vessels of a size and shape sufficient to allow diffusion or non-active transport of proteins, nutrients, and gas through the volume of the vessel and any other vessels in fluid communication with the vessel. In some embodiments, the vessel size is no more than about 500, 300, 200, 100, 10, 9, 8, 7, 6, 5, 4, 3, 2, or 1 microns in width or diameter. One of ordinary skill could determine how big of a vessel size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprising vessels of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that a polymer or metal coat the interior surface of one or more vessels and define a cylindrical or substantially cylindrical interior chamber or rectangular volume; and wherein the opening is positioned at one end of the vessel in order to facilitate manipulation or addition of reagents. In some embodiments, the base comprises one or a plurality of vessels of a size and shape sufficient to allow diffusion of protein, nutrients, and oxygen through the solid substrate in the presence of the cell culture medium. In some embodiments, the solid substrate comprises a plastic base with a vessel size of no more than 100 microns in diameter and comprises at least one layer of metal; metal can be functionalize to accept and to facilitate immobilization of the disclosed cells. In some embodiments, the bacterial cells are positioned within a vessel in solution and contained in suspension; and the base comprises a predetermined shape around which at least one coating or polymer physically adhere or chemically bond. In some embodiments, the solid substrate comprises at least one compartment defined at least in part by the shape of an interior surface of the solid substrate and accessible from a point outside of the solid substrate by an opening, optionally positioned at one end of the solid substrate or positioned within a pattern within the solid substrate. In embodiments, where the solid substrate comprises a hollow interior portion defined by at least one interior surface, the cells in suspension may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface solid substrate and encourages directional growth of the cells away from the opening. In the case of electrostimulus, the degree of containment and shape of the at least one compartment are conducive to position of at least one electrically conductive material and/or at least one electrode, the electrically conductive material capable of receiving and transmuting an electrical signal from the vessel to other equipment operably connected to the solid substrate and in an electrical circuit. In some embodiments, the vessel contains a first population of bacterial cells and at least one electrode is positioned proximate to the vessel or within the vessel at a distance from the cells sufficient to create electron flux in the vessel. In some embodiments, the electrode is placed from about 0.01 to about 15 millimeters from the population of bacterial cells. When and if electrodes are placed at to near the bacterial cells at a distance sufficient to conduct charge from the electrode to the bacterial cells, the charge can stimulate the bacterial cells to release their payload, which in some cases, can be a therapeutic molecule or signaling molecule. The signaling molecule may be able to create an electroactive molecule when oxidized or reduced, thereby allowing the bacterial cells to propagate a definitive and measurable electrical signal.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

As used herein, “patient in need thereof” or “subject in need thereof” refers to a living organism suffering from or prone to a disease or condition that can be treated by administration of at least one composition, vaccine or pharmaceutical composition disclosed herein, including, for example, a gastrointestinal disorder. Non-limiting examples include humans, other mammals, such as bovines, rats, mice, dogs, monkeys, goat, sheep, cows, deer, and other non-mammalian animals. In embodiments, a patient in need thereof or subject in need thereof is human.

The term “therapeutic effect” as used herein is meant to refer to some extent of relief of one or more of the symptoms of a disorder (e.g., a neoplasia or tumor) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a cancer or tumor). “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disorder. It is appreciated that, although not precluded, treating a disorder or condition does not require that the disorder, condition, or symptoms associated therewith be completely eliminated.

The terms “prophylaxis” or “prevention” means impeding the onset or recurrence of a disorder or one or more symptoms associated with a disorder.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although, in some embodiments, nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the nucleotide sequence encoding one or more antigens is free of modified nucleotide analogs. In some embodiments, the nucleotide sequence encoding one or more antigens comprises from about 1 to about 20 nucleic acid modifications. In some embodiments, the nucleotide sequence encoding one or more antigens comprises from about 1 to about 50 nucleic acid modifications. In some embodiments, the nucleotide sequence encoding one or more antigens independently comprise from about 1 to about 100 nucleic acid modifications.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also is a plasmid comprising one or more nucleotide sequences that encode one or a plurality of neoantigens. In some embodiments, the disclosure relates to a pharmaceutical composition comprising a first, second, third or more nucleic acid molecules, each of which encoding one or a plurality of neoantigens and at least one of each plasmid comprising one or more of the Formulae disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “conservative” amino acid substitutions may be defined as set out in Tables A, B, or C below. The vaccines, compositions, pharmaceutical compositions and method may comprise nucleic acid sequences comprising one or more conservative substitutions. In some embodiments, the compositions, devices and methods comprise nucleic acid sequences that retain from about 70% sequence identity to about 99% sequences identity to the sequence identification numbers disclosed herein but comprise one or more conservative substitutions. Conservative substitutions of the present disclosure include those wherein conservative substitutions (from either nucleic acid or amino acid sequences) have been introduced by modification of polynucleotides encoding polypeptides. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities to its target. In some embodiments, the target is a cell expressing β-catenin. Exemplary conservative substitutions are set out in Table A.

TABLE A
Conservative Substitutions I

| Side Chain Characteristics | Amino Acid |
|---|---|
| Aliphatic | |
| Non-polar | G A P I L V F |
| Polar - uncharged | C S T M N Q |
| Polar - charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | A L I V P |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the inhibitors described herein are intended to include nucleic acids and, where the inhibitors include polypeptide, polypeptides bearing one or more insertions, deletions, or substitutions, or any combination thereof, of amino acid residues as well as modifications other than insertions, deletions, or substitutions of amino acid residues.

“Exposing” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be an inducer as described herein (such as OxyR) and an electrical charge or ion. In some embodiments contacting includes allowing a signaling molecule described herein to interact with a protein or enzyme that is involved in a signaling pathway.

As used herein, “more than one” or “two or more” of the aforementioned amino acid substitutions means 2, 3, 4, 5, 6, 7, 8, 9, 10 or more of the recited amino acid or nucleic acid substitutions. In some embodiments, “more than one” means 2, 3, 4, or 5 of the recited amino acid substitutions or nucleic acid substitutions. In some embodiments, “more than one” means 2, 3, 4 or more of the recited amino acid substitutions or nucleic acid substitutions. In some embodiments, “more than one” means 2, 3 or 4 of the recited amino acid substitutions or nucleic acid substitutions. In some embodiments, “more than one” means 2 or more of the recited amino acid substitutions or nucleic acid substitutions. In some embodiments, “more than one” means 2 of the recited amino acid substitutions or nucleic acid substitutions.

The “percent identity” or “percent homology” (used interchangeably) of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length within a query sequence that either match or satisfy some positive-valued threshold score T when aligned with a word of the same length in a database sequence. T is referred to as the neighborhood word score threshold (Altschul et al., 1997). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The phrase “stringent hybridization conditions” or “stringent conditions” as used herein is meant to refer to conditions under which a nucleic acid molecule will hybridize another nucleic acid molecule, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g. 10 to 50 nucleotides) and at least about 600 C for longer probes, primers or oligonucleotides. Stringent conditions may also be achieved with the addition of destabilizing agents, such as formamide. In some of the disclosed methods, the bacterial cells are transformed with the disclosed plasmids or nucleic acid sequences. In some embodiments, the transformation step is achieved by subcloning the plasmid fragments in plasmids using PCR techniques that require stringent conditions of thermocycling.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least about 60%, about 80% or about 85%, and about 90%, about 95% or about 99% identical at the amino acid level or nucleic acid to the sequence used for comparison.

As used herein, the term “non-pathogenic” refers to a bacteria that is not capable of causing a disease or disorder in an animal when administered to an animal, including a human. In some embodiments, the microorganism is incapable of causing a disease or disorder when administered to a mammal. In some embodiments, the microorganism is a non-pathogenic microorganism incapable of causing a disease or disorder when administered to a human or domesticated animal (such as a dog, cat, horse, sheep, cow, goat, pig, etc.).

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence in the disclosed systems.

The disclosure relates to a composition comprising a vector. A “vector” is a nucleic acid molecule that can be used to introduce a nucleic acid sequence subcomponent into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), in the form of an RNA, DNA or hybrid RNA/DNA molecule comprising viral genome promoter sequences are operably linked to the expressible nucleotide sequence. In some embodiments, the expressible nucleotide sequence is introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

### Device and Systems

The present disclosure provides devices, methods, and systems involving production, maintenance, and electrostimulation of cells in microengineered configurations designed to manufacture and isolate recombinant protein, release recombinant protein or deliver protein or signaling molecules in an in vitro setting or to a localized component of a subject, such as a human. In some embodiments, the present disclosure relates to a device for production of recombinant protein or a device for release of recombinant protein in vivo, in which non-pathogenic bacterial cells are compartmentalized within a vessel of a device and stimulated by electricity, to release real-time signaling molecules or therapeutic molecules in an organism. In some embodiments, the device or system is free of a redox mediator.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 1, or a functional fragment thereof.

In some embodiments, a bacterial cell comprises a nucleic acid comprising at least 70%, at least about 75%, at least about 80%, at least about 85%, at least about 90%, at least about 95%, at least about 96%, at least about 97%, at least about 98% or at least about 99% sequence identity to SEQ ID NO: 2, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 3, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 4, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 5, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 6, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 7, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 8, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 9, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 10, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 11, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 12, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 13, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 14, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 15, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 16, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 17, or a functional fragment thereof.

In some embodiments, a bacterial cell comprises a nucleic acid comprising at least about 70%, at least about 75%, at least about 80%, at least about 85%, at least about 90%, at least about 95%, at least about 96%, at least about 97%, at least about 98% or at least about 99% sequence identity to SEQ ID NO: 18 or a functional fragment thereof.

The disclosure relates to a device, the device comprising one or a series of vessels or wells comprising at least a first bacterial cell population. In some embodiments, the first bacterial cell population comprising a bacterial cell comprising a nucleic acid molecule or molecules, wherein the nucleic acid molecule or molecule comprise: (i) a first nucleic acid sequence comprising a first promoter operably linked to a second nucleic acid sequence encoding an inducer; and (ii) a third nucleic acid sequence comprising a second promoter operably linked to a fourth nucleic acid sequence encoding a therapeutic sequence or a signaling sequence. In some embodiments the first nucleic acid sequence comprises at least about 70%, at least about 75%, at least about 80%, at least about 85%, at least about 90%, at least about 95%, at least about 96%, at least about 97%, at least about 98% or at least about 99% homology to SEQ ID NO: 1 or a functional fragment thereof and the second nucleic acid encoding the inducer comprises at least about 70%, at least about 75%, at least about 80%, at least about 85%, at least about 90%, at least about 95%, at least about 96%, at least about 97%, at least about 98% or at least about 99% homology to SEQ ID NO: 9 or a functional fragment thereof. In some embodiments, the promoter encoding the inducer is constitutively active. In some embodiments, the third nucleic acid sequence comprises at least about 70%, at least about 75%, at least about 80%, at least about 85%, at least about 90%, at least about 95%, at least about 96%, at least about 97%, at least about 98% or at least about 99% homology to SEQ ID NO: 2 or a functional fragment thereof. In some embodiments, the therapeutic molecule is a cytokine.

Some embodiments include a system. The system can comprise a bioreactor operable to vitally support one or more microorganisms. Meanwhile, the bioreactor can comprise a bioreactor cavity means for containing the microorganism(s) and a fluidic support medium, a parameter sensing means for monitoring a cavity environment condition at the bioreactor, and a bioreactor mixing means for mixing the microorganism(s). Further, the bioreactor can be autoclaved one or more times to sterilize the bioreactor, and the bioreactor can be folded up and/or rolled up. In some embodiments, the microorganism(s) can comprise at least one bacterial cell disclosed herein. In some embodiments, the bioreactor can comprise an organic carbon material delivery means for supplying organic carbon material to the microorganism(s). In these or other embodiments, the bioreactor can comprise a pressure regulation means for limiting a bioreactor cavity pressure of the bioreactor cavity. In these or other embodiments, the bioreactor can comprise a filtration means for filtering a supply of at least one of a gas, a nutritional media, or the fluidic support medium. In these or other embodiments, the bioreactor is configured so that the bioreactor is able to be autoclaved to sterilize the bioreactor before the bioreactor vitally supports the microorganism(s). In these or other embodiments, the bioreactor can be operable to vitally support one or more first microorganisms of the microorganism(s) and one or more second microorganisms of the microorganism(s) at different times, and the bioreactor can be autoclaved to sterilize the bioreactor after the bioreactor vitally supports the first microorganism(s) and before the bioreactor vitally supports the second microorganism(s).

Some embodiments include a system. The system can comprise a support structure operable to mechanically support a first bioreactor. The support structure can comprise a first frame and a second frame together being operable to mechanically support the first bioreactor in interposition between the first frame and the second frame. The first frame can maintain a first set point temperature of the first bioreactor when the first bioreactor is vitally supporting one or more first microorganisms and when the support structure is mechanically supporting the first bioreactor. Further, the first bioreactor can be operable to vitally support the first microorganism(s). In some embodiments, the first bioreactor can comprise a first bioreactor cavity configured to contain the first microorganism(s) and a first fluidic support medium, and can comprise one or more first bioreactor walls at least partially forming the first bioreactor cavity. Also, the first bioreactor wall(s) can comprise at least one first bioreactor wall material and the at least one first bioreactor wall material can be flexible.

In some embodiments, the system can comprise the first bioreactor, the first bioreactor can be autoclaved one or more times to sterilize the first bioreactor, and/or the first bioreactor can be folded up and/or rolled up. In these or other embodiments, the first bioreactor can comprise one or more bioreactor fittings in communication with the first bioreactor cavity, one or more gas delivery devices disposed within the first bioreactor cavity, and one or more flexible tubes disposed within the first bioreactor cavity. Further, the bioreactor fitting(s) can comprise at least one gas delivery fitting, the gas delivery device(s) can inject gas into the first bioreactor cavity to mix the first microorganism(s), and the flexible tube(s) can comprise at least one gas delivery tube coupling the gas delivery device(s) to the gas delivery fitting(s). In these or other embodiments, the first microorganism(s) is a bacterial cell disclosed herein comprising at least one promoter responsive to an electrochemical signal, such as presence of an oxidized form of OxyR. In these or other embodiments, the second frame can maintain the first set point temperature of the first bioreactor when the first bioreactor is vitally supporting the first microorganism(s) and when the support structure is mechanically supporting the first bioreactor. In these or other embodiments, the first frame can comprise two or more first frame rails, each first frame rail of the two or more first frame rails can comprise a first frame rail conduit, each first frame rail conduit of the two or more first frame rail conduits can convey a temperature maintenance fluid to exchange thermal energy between the first bioreactor and the temperature maintenance fluid in order to maintain the first set point temperature of the first bioreactor when the first bioreactor is vitally supporting the first microorganism(s), and the two or more first frame rails can mechanically support the first bioreactor. In these or other embodiments, the two or more first frame rails can receive the temperature maintenance fluid in parallel. In these or other embodiments, the two or more first frame rails can comprise stainless steel, and the temperature maintenance fluid can comprise water. In these or other embodiments, the support structure can comprise a floor gap underneath one of the first frame or the second frame to permit the first bioreactor to bulge into the floor gap when the support structure is mechanically supporting the first bioreactor. In these or other embodiments, the system can further comprise at least one light source mechanically supported by the support structure and operable to illuminate the first microorganism(s) when the first bioreactor is vitally supporting the first microorganism(s) and when the support structure is mechanically supporting the first bioreactor. In some embodiments, the bioreactor comprises an electrode at or proximate to the culture of bacterial cells.

In some embodiments, the bacterial cells express no less than about 3×10⁻⁷ micrograms per bacterial cell. In some embodiments, the bacterial cells express no less than about 3.7×10⁻⁷ micrograms per bacterial cell. In some embodiments, the bacterial cells express no less than about 3.8×10⁻⁷ micrograms per bacterial cell. In some embodiments, the bacterial cells express no less than about 3.9×10⁻⁷ micrograms per bacterial cell. In some embodiments, the bacterial cells express no less than about 4.0×10⁻⁷ micrograms per bacterial cell. In some embodiments, the bacterial cells express no less than about 4.1×10⁻⁷ micrograms per bacterial cell. In some embodiments, the bacterial cells express no less than about 4.5×10⁻⁷ micrograms per bacterial cell. In some embodiments, the bacterial cells express no less than about 4.7×10⁻⁷ micrograms/per cell.

Smaller devices such as a micro-sized bioreactors are contemplated by this disclosure. In some embodiments, the disclosure relates to a device comprising a vessel comprising one or more populations of bacterial cells can be manufactured and, if desired, implanted into an organism. In some embodiments, the device comprises a bacterial population from E. coli that are

In some embodiments, the device is a capsule comprising a closed bacterial system, wherein the capsule comprises a biocompatible surface that enables diffusion across the surface. In some embodiments, the capsule comprises a cavity of about 100 microliters, about 120 microliters, 130 microliters, or 200 microliters or more, wherein the cavity comprises one, two, three or more bacterial cell populations disclosed herein.

In some embodiments, and under exposure to an inducer, and at an optical density of about 1, at least one of the bacterial cell populations would produce about 0.02, 0.03, 0.0350, 0.0375 mg every 24 hours. In some embodiments, after exposure to one or more stimuli, the capsule would diffuse or release from about 0.01 mg to about 0.1 mg every 24 hours. In some embodiments, after exposure to one or more stimuli, the capsule would diffuse or release from about 0.005 mg to about 0.5 mg every 24 hours.

### Inducers

The disclosure relates to a device or system comprising a solid support or substrate that comprises vessels or wells comprising one or a plurality of bacterial cells. In some embodiments, the one or plurality of bacterial cells comprise an inducible promoter responsive to an inducer. Inducers using the system may be Hydrogen peroxide and peroxide. In some embodiments, methods in which the methods that involve a step of electrostimulation comprise a step of exposing the bacterial cell or plurality of cells to oxygen or peroxide. In some embodiments, the system or device comprises the presence of an autoinducer such as below:

### Autoinducer Structures

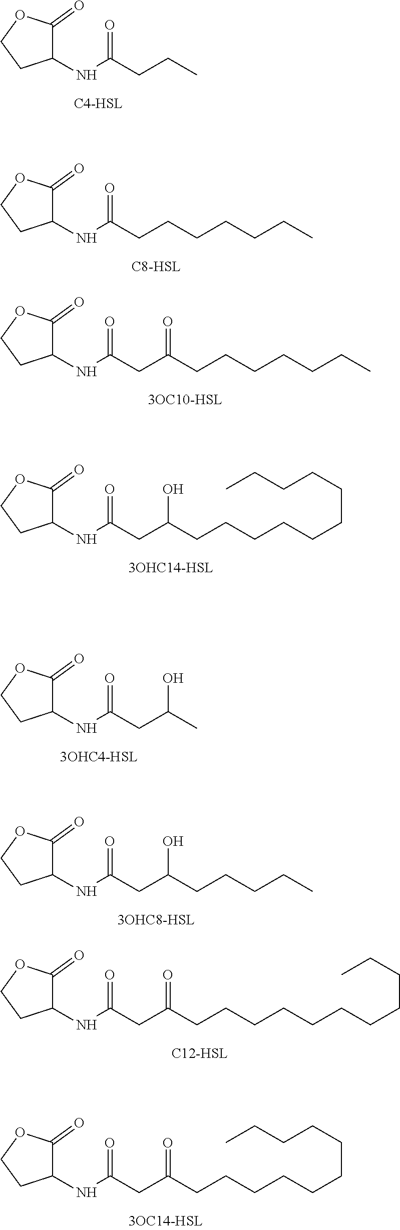

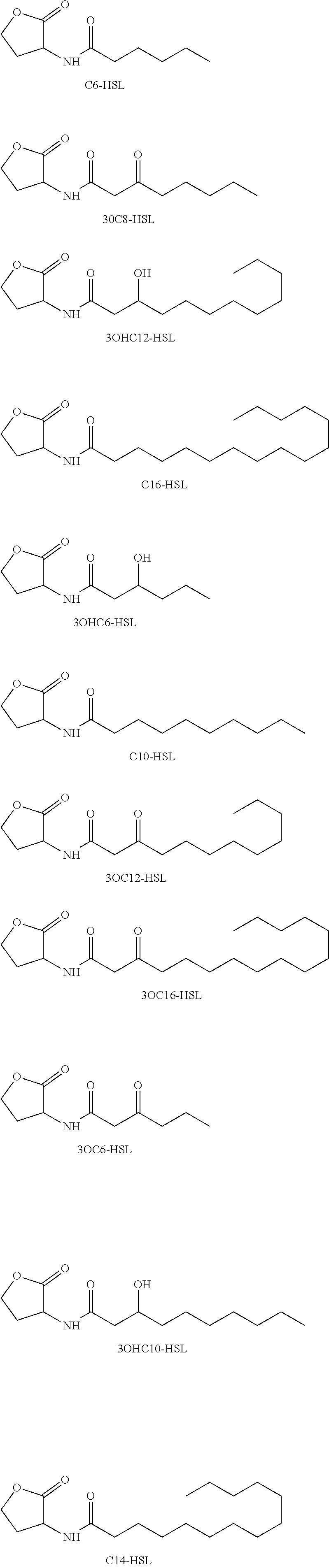

### Methods

The disclosure provides for methods of producing a recombinant amino acid in one or a plurality of bacterial cells in a culture vessel comprising a solid substrate, said method comprising: (a) contacting a first bacterial cell or a first population of isolated bacterial cells with the solid substrate, said substrate comprising at least one exterior surface, at least one interior surface and at least one interior chamber defined by the interior surface and at least one opening; (b) applying a cell medium into the culture vessel with a volume of cell medium sufficient to cover the at least one interior chamber; and (c) exposing the first bacterial cells or first bacterial cell population with an inducer for a time period sufficient to stimulate expression of the recombinant amino acid; wherein the first cell or first population of cells comprises a nucleic acid molecule comprising an expressible nucleic acid sequence encoding the amino acid operably linked to a regulatory sequence specific for association with the inducer. In some embodiments, the method further comprises exposing the first bacterial cells or first bacterial cell population to a second stimulus to express a transmembrane domain protein in a sufficient amount to disrupt the cell wall of the bacteria and release the recombinant amino acid.

For purposes of therapy, the device disclosed herein can be utilizes as an implant in an organism, such as a human. The solid substrate can house or compartmentalize bacterial cells in the vessels or wells within the disclosed device. The device can be made of biocompatible plastic or biocompatible material other than plastic and implanted at a site of administration. As an example, the disclosure relates to a method of treating a gastrointestinal disorder in a subject by inducing a first bacterial cell population to an inducer nested on a device. If the bacterial cells and the well within they sit or are immobilized are part of an electrical circuit, methods of treating a subject can be performed by inducing expression and inducing release of a therapeutic molecule encoded by a nucleic acid operably linked to the pOxyR or pOxyS promoter sequences. In this way bacterial cells which are a component of the disclosed device may be exposed to electrical charge or voltage drops and release the therapeutic molecules in therapeutically effective amounts. In some embodiments, methods of treatment of a subject comprise administering to a subject comprising the disclosed device an electrostimulation of a magnitude sufficient to cause release of a therapeutically effective amount of a therapeutic molecule. In some embodiments, the therapeutic molecule is a cytokine, such as GMCSF. In some embodiments, the cytokine is IL-10, IL-16, IL-17, IL-12 or IL-2. In some embodiments, the therapeutic molecule is any therapeutic molecule disclosed in Table Y.

The disclosure further relates to a method of treating Crohn's disease in a subject comprising administering to a subject in need of treatment an electrostimulation sufficient to oxidize OxyR species in a first population of bacterial cells, such that the bacterial cells release a therapeutically effective amount of therapeutic molecule, such as GMCSF, into the subject. In some embodiments, the first population of bacterial cells comprise a pOxyR promoter sequence operably linked to the nucleic acid sequence that encodes GMCSF or a functional fragment or functional variant thereof. The disclosure further relates to a method of treating a disorder or disease of Table Y in a subject comprising administering to a subject in need of treatment an electrostimulation sufficient to oxidize OxyR in a first population of bacterial cells, such that the bacterial cells release a therapeutically effective amount of therapeutic molecule in the presence of an oxidized OxyR species. In such methods the first bacterial population comprises a vector comprising a nucleic acid sequence encoding the therapeutic molecule or amino acid and a regulatory sequence operably linked to the nucleic acid sequence encoding the therapeutic molecule. In some embodiments, the first bacterial population comprises a nucleic acid sequence that encodes OxyR and a nucleic acid sequence encoding the therapeutic molecule operably linked to the pOxyS promoter, respectively. In some embodiments, the device or composition disclosed herein comprises a first population of bacterial cells that comprise a nucleic acid sequence encoding OxyR and a second bacterial population that comprises a nucleic acid sequence encoding the therapeutic molecule operably linked to the a pOxyS promoter. In such a non-limiting example, the one bacterial cell population constitutively expresses the OxyR protein, an electrical stimulus creates oxidized species of the OxyR protein, and the oxidized OxyR activates the pOxyS promoter of the second population, which in turn causes expression of the therapeutic protein. Under conditions in which the bacterial population disrupts its cell walls, the population encoding a therapeutic molecule can be released into its microenvironment. The microenvironment may by an in vitro system or bioreactor used to manufacture and, optionally, isolate a recombinant protein in batches or deliver such a molecule into an organism.

In some embodiments, the disclosure relates to a pharmaceutical composition comprising a therapeutic molecule or signaling molecule disclosed herein. In some embodiments, the pharmaceutical composition comprises a therapeutically effective dose of the at least one bacterial cell. As used herein the terms “therapeutically effective dose” means the number of cells per dose administered to a subject in need thereof sufficient in number to ameliorate or reduce the burden of disease symptoms of the subject. In some embodiments, the terms “therapeutically effective dose” means the number of cells per dose administered to a subject in need thereof sufficient in number to reduce the symptoms of a gastrointestinal disorder.

In some embodiments, the pharmaceutical composition disclosed herein, wherein the therapeutically effective dose is from about 1×10⁴ to about 1×10⁷ bacterial cells. In some embodiments, the therapeutically effective dose is from about 1×10¹ to about 1×10⁷ bacterial cells. In some embodiments, the therapeutically effective dose is from about 1×10² to about 1×10⁷ bacterial cells. In some embodiments, the therapeutically effective dose is from about 1×10³ to about 1×10⁷ bacterial cells. In some embodiments, the therapeutically effective dose is from about 1×10⁴ to about 1×10⁷ bacterial cells. In some embodiments, the therapeutically effective dose is from about 1×10⁵ to about 1×10⁷ bacterial cells. In some embodiments, the therapeutically effective dose is from about 1×10⁶ to about 1×10⁷ bacterial cells.

In some embodiments, the therapeutically effective dose is from about 1×10¹ to about 1×10⁶ bacterial cells. In some embodiments, the therapeutically effective dose is from about 1×10² to about 1×10⁶ bacterial cells. In some embodiments, the therapeutically effective dose is from about 1×10³ to about 1×10⁶ bacterial cells. In some embodiments, the therapeutically effective dose is from about 1×10⁴ to about 1×10⁶ bacterial cells. In some embodiments, the therapeutically effective dose is from about 1×10⁵ to about 1×10⁶ bacterial cells.

In some embodiments, the therapeutically effective dose is from about 1×10¹ to about 1×10⁵ bacterial cells. In some embodiments, the therapeutically effective dose is from about 1×10² to about 1×10⁵ bacterial cells. In some embodiments, the therapeutically effective dose is from about 1×10³ to about 1×10⁵ bacterial cells. In some embodiments, the therapeutically effective dose is from about 1×10⁴ to about 1×10⁵ bacterial cells. In some embodiments, the therapeutically effective dose is from about 1×10¹ to about 1×10⁴ bacterial cells. In some embodiments, the therapeutically effective dose is from about 1×10² to about 1×10⁴ bacterial cells. In some embodiments, the therapeutically effective dose is from about 1×10¹ to about 1×10³ bacterial cells. In some embodiments, the therapeutically effective dose is from about 1×10³ to about 1×10⁴ bacterial cells. In some embodiments, the therapeutically effective dose is from about 1×10² to about 1×10³ bacterial cells. In some embodiments, the therapeutically effective dose is from about 1×10¹ to about 1×10² bacterial cells. In some embodiments, the therapeutically effective dose is from about 1×10^{o} to about 1×10² bacterial cells. In some embodiments, the therapeutically effective dose is from about 1×10^{o} to about 1×10⁸ bacterial cells. In some embodiments, the therapeutically effective dose is from about 1×10^{o} to about 1×10⁹ bacterial cells. In some embodiments, the therapeutically effective dose of bacterial cells is more than 1×10⁹ bacterial cells. In some embodiments, the therapeutically effective dose of bacterial cells is less than 10 cells.

In some embodiments, the composition or device disclosed herein comprises at least one non-pathogenic bacterial cell, wherein the non-pathogenic bacterial cell comprises at least a first and a second nucleic acid sequence, the first nucleic acid sequence comprising at least one non-constitutive promoter operably linked to the second nucleic acid sequence, the second nucleic acid encoding at least one inducer, wherein the non-constitutive promoter is an inducible promoter responsive to the responsive to at least one stimuli and the at least one stimuli comprises the presence of a certain density or a certain number of bacterial cells comprising the first and second nucleic acid sequences. In some embodiments, the bacterial cells comprise at least two inducible promoters, a first promoter responsive to the presence of a certain density or certain number of bacterial cells and a second promoter, such as the pOxyS promoter responsive to the presence of oxidized species of protein. In any such devices or methods in which such devices comprise such bacterial cells, electrostimulation causes expression of a therapeutic molecule or signaling molecule operably linked to the second promoter, and secretion of the therapeutic molecule is achieved by expression of a pore-forming protein in the bacterial cell wall, such as TolAIII, which is operably linked to the first promoter.

In some embodiments, the therapeutic or signaling molecules are the following:

GMCSF sequence (a therapeutic molecule)
MKKTAIAIAVALAGFATVAQAAAMAAPARSPSPSTQPWEHVNAIQEARRL
LNLSRDTAAEMNETVEVISEMFDLQEPTCLQTRLELYKQGLRGSLTKLKG
PLTMMASHYKQHCPPTPETSCATQIITFESFKENLKDFLLVIPFDCWEPV
QEAAAGSEQKLISEEDLKASGADHHHHHH
DsRed Express II sequence (a biomarker)
MDSTENVIKPFMRFKVHMEGSVNGHEFEIEGEGEGKPYEGTQTAKLQVTK
GGPLPFAWDILSPQFQYGSKVYVKHPADIPDYKKLSFPEGFKWERVMNFE
DGGVVTVTQDSSLQDGTFIYHVKFIGVNFPSDGPVMQKKTLGWEPSTERL
YPRDGVLKGEIHKALKLKGGGHYLVEFKSIYMAKKPVKLPGYYYVDSKLD
ITSHNEDYTVVEQYERAEARHHLFQ

### Electrodes

In some embodiments, the device, system disclosed herein comprise one or more electrodes. In some embodiments, the one or more electrodes transmit current variation generated by the reaction between the bacterial cells and inducers that are added to the system or recombinantly produced by at least one bacterial cell population in the device or system. In some embodiments, the electrode transmits current variation generated by a battery source to equipment necessary to provide a readout of the stimuli of which the present promoter sequences are responsive. For instance, in the case of a spectrophotometer to measure absorbance of a reactant vessel in the device. In some embodiments, the electrodes comprise metal. In some embodiments, the electrodes comprise a Ag/AgCl alloy. In some embodiments, the electrode is a carbon scaffold upon which a metal is deposited. In some embodiments, the electrodes comprise a carbon scaffold of carbon nanotubes. Electrode structures which are suitable for the present disclosure and methods for the production of such structures have already been suggested in biosensor technology for other purposes. In this regard, reference is made to U.S. Pat. No. 6,645,359 and its content is incorporated herein by reference in its entirety. Electrodes or Electrically conductive tracks are created or isolated on first surface. Tracks represent the electrodes of biosensor. As used herein, the phrase “electrode set” is a set of at least two electrodes, for example, about 2 to about 200, or from about 3 to about 10 electrodes. These electrodes may, for example, be a working (or measuring) electrode and an auxiliary electrode. In some embodiments, tracks cooperate to form an interdigitated electrode array positioned within the periphery of recesses and leads that extend from array and between recesses or vessels toward end. In some embodiments, the device or composition comprises a solid substrate with at least two vessels into which bacterial cells are seeded and electrodes are positioned. Tracks are constructed from electrically conductive materials. Non-limiting examples of electrically-conductive materials include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys, oxides, or metallic compounds of these elements.

Preferably, tracks include gold, platinum, palladium, iridium, or alloys of these metals, since such noble metals and their alloys are unreactive in biological systems. In some embodiments, the track is a working electrode made of silver and/or silver chloride, and track is an auxiliary electrode that is also made of silver and/or silver chloride and is substantially the same size as the working electrode.

Tracks are isolated from the rest of the electrically conductive surface by laser ablation. Techniques for forming electrodes on a surface using laser ablation are known. Techniques for forming electrodes on a surface using laser ablation are known. See, for example, U.S. patent application Ser. No. 09/411,940, filed Oct. 4, 1999, and entitled “LASER DEFINED FEATURES FOR PATTERNED LAMINATES AND ELECTRODE”, the disclosure of which is expressly incorporated herein by reference. Tracks are preferably created by removing the electrically conductive material from an area extending around the electrodes. Therefore, tracks are isolated from the rest of the electrically-conductive material on a surface by a gap having a width of about 5 microns to about 500 microns, preferably the gap has a width of about 100 microns to about 200 microns.

Alternatively, it is appreciated that tracks may be created by laser ablation alone on bottom substrate. Further, tracks may be laminated, screen-printed, or formed by photolithography. Multi-electrode arrangements are also possible in accordance with this disclosure. For example, it is contemplated that a biosensor may be formed that includes an additional electrically conductive track. In a three-electrode arrangement, the first track is a working electrode, the second is a counter electrode, and the third electrode is a reference electrode. It is also appreciated that an alternative three-electrode arrangement is possible where tracks are working electrodes and a third electrode is provided as an auxiliary or reference electrode. It is appreciated that the number of tracks, as well as the spacing between tracks in array may vary in accordance with this disclosure and that a number of arrays may be formed as will be appreciated by one of skill in the art. In some embodiments, the electrodes are embedded on or attached to a solid support or solid substrate and part of an electric circuit that connects a display, a computer storage memory, and a computer processor to the device.

Micro-electrode arrays are structures generally having two electrodes of very small dimensions, typically with each electrode having a common element and electrode elements or micro-electrodes. If “interdigitated” the arrays are arranged in an alternating, finger-like fashion (See, e.g., U.S. Pat. No. 5,670,031). These are a sub-class of micro-electrodes in general. Interdigitated arrays of micro-electrodes, or IDAs, can exhibit desired performance characteristics; for example, due to their small dimensions, IDAs can exhibit excellent signal to noise ratios as calculated by the disclosed algorithm.

Interdigitated arrays have been disposed on non-flexible substrates such as silicon or glass substrates, using integrated circuit photolithography methods. ID As have been used on non-flexible substrates because ID As have been considered to offer superior performance properties when used at very small dimensions, e.g., with feature dimensions in the 1-3 micrometer range. At such small dimensions, the surface structure of a substrate (e.g., the flatness or roughness) becomes significant in the performance of the IDA. Because non-flexible substrates, especially silicon, can be processed to an exceptionally smooth, flat, surface, these have been used with IDAs. In some embodiments, the at least one electrode is a component of any IDA disclosed herein.

In some embodiments, the disclosure relates device comprising an array with multiple electrodes at or proximate to one or more arrayed vessels within a device. In some embodiments, each vessel in which the bacterial cells are positioned and cultured are in fluid communication with one another, such that inducers and sources of electrostimulation can address the multiple vessels selectively. In the embodiments that involve stimulation of electricity or current, at least one electrode operably connected to a power source such as a battery can elicit an electric signal through the cell culture medium and create charge for an inducer such as OxyR, converting the OxyR into an oxidized species.

In some embodiments, the electrodes that provide an electrical stimulus for the electrosensitive bacterial cells are position from about 0.1 to about 10 millimeters from the cell medium or immobilized bacterial cells. In some embodiments the device comprises at least two electrodes, an anode and cathode, positioned across the culture well or vessel electronically and operably connect to a circuit from which a power source create a current.

Devices and compositions of the disclosure relate to devices or compositions that comprise one, two or three bacterial cells populations. The bacterial cells or bacterial cell populations may comprise two, three, four or more exogenous nucleic acid sequences. In some embodiments, the bacterial cells comprise a first nucleic acid sequence encoding an inducer, such as OxyR or a functional fragment thereof, operably linked to a second nucleic acid sequence that is a constitutive promoter. In some embodiments, the constitutive promoter is proD or SEQ ID NO:1, or a functional fragment thereof. Some embodiments include a device comprising bacterial cells comprise a first nucleic acid sequence encoding an inducer, such as OxyR or a functional fragment thereof, operably linked to a second nucleic acid sequence that is a constitutive promoter; a third nucleic acid sequence encoding a therapeutic molecule or signaling molecule operably linked to a four nucleic acid sequence that is a second promoter. In some embodiments, the second promoter is a pOxyS promoter or functional fragment thereof. In some embodiments, the bacterial cell or bacterial cell populations comprise nucleic acid sequences disclosed in Table X or functional fragments of the nucleic acid sequences disclosed in Table X.

TABLE X
Organism Table

| | | Inducer | Expression | Functional |
|---|---|---|---|---|
| Strain Name | SEQ IDs | input | output | effect |
| Escherichia coli | SEQ ID NO. 20 + | Hydrogen | Constitutive | Continuous |
| for electrogenetically- | SEQ ID NO. 1 + | peroxide | expression of an | electrode |
| activated signal routing | SEQ ID NO. 2 + | | adhesion domain | immobilization |
| | SEQ ID NO. 3 + | | (AIDAc) fused | capability via |
| | SEQ. ID NO. 9 + | | to gold-binding | AIDAc |
| | SEQ ID NO. 11 + | | peptide (GBP₃) | embedding in |
| | SEQ ID NO. 17; | | and His6 peptide; | outer membrane |
| | SEQ ID NO. 21 + | | Peroxide- | for surface |
| | SEQ ID NO. 4 + | | induced LasI | display of gold- |
| | SEQ ID NO. 5 + | | AHL synthase | affinity peptide; |
| | SEQ ID NO. 6 + | | with LAA | peroxide- |
| | SEQ ID NO. 7 + | | degradation tag | triggered |
| | SEQ ID NO. 8 | | | transduction of |
| | | | | quorum sensing |
| | | | | signal AHL, |
| | | | | which can be |
| | | | | electrochemically |
| | | | | controlled. |
| Escherichia coli | SEQ ID NO. 20 + | Hydrogen | Constitutive | Continuous |
| for electrogenetically- | SEQ ID NO. 1 + | peroxide | expression of an | electrode |
| activated fluorescence | SEQ ID NO. 2 + | | adhesion domain | immobilization |
| | SEQ ID NO. 3 + | | (AIDAc) fused | capability via |
| | SEQ. ID NO. 9 + | | to a gold- | AIDAc |
| | SEQ ID NO. 10 + | | binding peptide | embedding in |
| | SEQ ID NO. 17; | | (GBP₃); | outer membrane |
| | SEQ ID NO. 21 + | | Peroxide-induced | for surface |
| | SEQ ID NO. 4 + | | Superfolder | display of gold- |
| | SEQ ID NO. 5 + | | green | affinity peptide; |
| | SEQ ID NO. 6 + | | fluorescent | peroxide- |
| | SEQ ID NO. 7 + | | protein with | triggered |
| | SEQ ID NO. 8 | | degradation tag | production of |
| | | | (sfGFP-LAA) | transient |
| | | | | fluorescence, |
| | | | | which can be |
| | | | | electrochemically |
| | | | | controlled. |
| Escherichia coli | SEQ ID NO. 20 + | Hydrogen | Constitutive | Continuous |
| for electrogenetically- | SEQ ID NO. 1 + | peroxide | expression of an | electrode |
| activated quantification | SEQ ID NO. 2 + | | adhesion domain | immobilization |
| of expression level | SEQ ID NO. 3 + | | (AIDAc) fused | capability via |
| | SEQ. ID NO. 9 + | | to a gold- | AIDAc |
| | SEQ ID NO. 12 + | | binding peptide | embedding in |
| | SEQ ID NO. 17; | | (GBP₃); | outer membrane |
| | SEQ ID NO. 21 + | | Peroxide- | for surface |
| | SEQ ID NO. 4 + | | induced β- | display of gold- |
| | SEQ ID NO. 5 + | | galactosidase | affinity peptide; |
| | SEQ ID NO. 6 + | | enzyme with LAA | peroxide- |
| | SEQ ID NO. 7 + | | degradation tag | triggered β-gal |
| | SEQ ID NO. 8 | | | catalysis of |
| | | | | substrates into |
| | | | | chromogenic |
| | | | | products, which |
| | | | | can be |
| | | | | electrochemically |
| | | | | controlled. |
| Escherichia coli | SEQ ID NO. 22 + | Acyl homoserine | β-galactosidase | β-galactosidase- |
| AHL-actuated strain for | SEQ ID NO. 12 + | lactone (AHL): | enzyme | catalyzed |
| electrochemical signal | SEQ ID NO. 13 + | N-3-oxo- | | cleavage of redox |
| verification | SEQ ID NO. 14 + | dodecanoyl-L- | | mediator |
| | SEQ ID NO. 15 | Homoserine | | substrate (PAPG) |
| | | lactone | | into readily- |
| | | | | oxidized product |
| | | | | (PAP(r)) for |
| | | | | electrochemical |
| | | | | detection |
| Escherichia coli | SEQ ID NO. 22 + | Acyl homoserine | Constitutive | Porous cell |
| AHL-actuated strain with | SEQ ID NO. 13 + | lactone (AHL): | DSRedExpress2 | barrier due to |
| pump-then-burst capability | SEQ ID NO. 14 + | N-3-oxo- | production; | TolAIII pore |
| | SEQ ID NO. 15 + | dodecanoyl-L- | AHL-induced | embedded in |
| | SEQ ID NO. 15 + | Homoserine | granulocyte | membrane; |
| | SEQ ID NO. 18 + | lactone | macrophage | extracellular |
| | SEQ ID NO. 19 | | cell stimulating | passage of |
| | | | factor (GMCSF) | expressed |
| | | | and TolAIII | proteins |
| | | | membrane pore | (GMCSF, |
| | | | | DsRedExpress2) |

TABLE Y
Applications of Engineered Strains/Communities

| | Disorder | | |
|---|---|---|---|
| Strain(s) | Treated/Application | Inducer | Product Released or Secreted |
| E. coli pAHL-GMCSF | Mechanism for | Acyl homoserine | dsRedExpressII expressed at |
| | rapid bacterial | lactone (AHL) | high levels intracellularly. AI- |
| | secretion of high | (induced from about | 1 induces formation of porins |
| | titer recombinant | 100-500 nM of AHL | causing rapid release of |
| | proteins. | in solution) | dsRedExpressII. |
| | Architecture | | High levels (greater than 1.5 |
| | includes | | mg/mL dsRedExpressII) |
| | intracellular | | accumulate in cultures. |
| | expression of | | We estimate that expression |
| | dsRedExpressII (a | | of a recombinant protein in |
| | representative | | place of dsRedExpressII |
| | recombinant | | could produce from about |
| | protein) and | | 0.01 to about 0.1 mg per cell |
| | inducible | | in a 100 uL capsule (see |
| | expression of a | | calculations below). This |
| | porin protein | | would be compared to dosing |
| | allowing secretion | | of 8 μg/kg per day used in |
| | of dsRedExpressII. | | human study (0.56 mg per |
| | | | day for a 70 kg person) |
| | | | (Dieckgraefe B K, Korzenik |
| | | | J R. Treatment of active |
| | | | Crohn's disease with |
| | | | recombinant human |
| | | | granulocyte-macrophage |
| | | | colony-stimulating |
| | | | factor. Lancet 2002; 360: 1478- |
| | | | 1480). |
| E. coli pOxyR-lasI-laa + | Chrohn's Disease | Electrodes that | GMCSF released as |
| E. coli pAHL-LacZ + | User-controlled, | generate hydrogen | therapeutic. Betagalactosidase |
| E. coli pAHL-GMCSF | electronic (remote) | peroxide. | (lacZ) released and measured |
| In a capsule or device | activation of GM- | | using electrodes to confirm |
| with electrodes | CSF therapeutic | | successful induction and |
| | production. | | activation of system. |
| | Includes | | |
| | mechanism for | | |
| | electronic feedback | | |
| | that activation was | | |
| | completed. | | |
| E. coli pOxyR-lasI-laa + | Treatment of | Electrode that | Range of possible |
| E. coli pAHL-LacZ + | disorders in the | generates hydrogen | therapeutics |
| E. coli pAHL-Therapeutic | digestive system by | peroxide | (see table below). |
| In a capsule or device | release of a | | |
| with electrodes | molecular | | |
| | therapeutic (in | | |
| | place of GM-CSF | | |
| | above) for possible | | |
| | treatment of a range | | |
| | of conditions (see | | |
| | table below). | | |
| E. coli pOxyR-lasI-laa | Modulation of Host | Electrodes that | AHL (3OC12HSL) signaling |
| In a capsule or device | Microbiome | generate hydrogen | molecule (from about 20 to |
| with electrodes | through generation | peroxide. | about 300 nM AHL generated |
| | of signaling | | in solution) by cells adhered |
| | molecules. | | to electrode. This is on par |
| | | | with the amount generated |
| | | | naturally by PAO1 |
| | | | (Pseudomonas aeruginosa) |
| | | | and in theory could modulate |
| | | | microbiome. |
| E. coli pOxyR- | Modulation of Host | Electrodes that | Potential Signaling |
| SignalingMolecule | Microbiome though | generate hydrogen | Molecules for Release: |
| In a capsule or device | generation of | peroxide. | Autoinducer-2 |
| with electrodes | signaling molecules | | Additional AHL molecules |
| | (other than | | Redox active molecules, for |
| | 3OC12HSL as | | instance pyocyanin |
| | above). Could | | |
| | include a range of | | |
| | molecules. | | |
| | Note that | | |
| | microbiome could | | |
| | include human GI | | |
| | tract microbiome or | | |
| | others such as the | | |
| | plant microbiome. | | |

### Additional Applications for Disease Treatment by Replacing GM-CSF:

| | | Therapeutic range or | |
|---|---|---|---|
| | Therapeutic molecule/protein | concentration | |
| Disease | produced | produced | Reference |
| Phenylketonuria | Enzymes for phenylalanine | Maximal activity | Isabella et al. 2018 |
| | (Phe) degradation to | achieved by | https://doi.org/10.1038/nbt. |
| | phenylpyruvate (PP) and | probiotic: | 4222 |
| | trans-cinnamate (TCA): | 2.88 ± 0.05 and | |
| | - phenylalanine | 13.68 ± 0.56 μmol | |
| | ammonia lyase | per h per 10° cells for | |
| | - Phe importer PheP | TCA and PP | |
| | - L-amino acid | respectively | |
| | deaminase | | |
| IBD | IL-27 (sequence: | While treatment with | Hanson 2015 |
| | MKKKIISAILMSTVILSAAAPLSG | recombinant IL-27 (1 | https://doi.org/10.1053%2F |
| | VYAGYTETALVALSQPRVQCHA | mg or 500 ng) had no | j.gastro.2013.09.060 |
| | SRYPVAVDCSWTPLQAPNSTRST | effect on disease | |
| | SFIATYRLGVATQQQSQPCLQRS | activity index (DAI), | |
| | PQASRCTIPDVHLFSTVPYMLNV | IL27-expressing cells | |
| | TAVHPGGASSSLLAFVAERIIKPD | decreased DAI by | |
| | PPEGVRLRTAGQRLQVLWHPPA | half | |
| | SWPFPDIFSLKYRLRYRRRGASH | | |
| | FRQVGPIEATTFTLRNSKPHAKY | | |
| | CIQVSAQDLTDYGKPSDWSLPG | | |
| | QVESAPHKPSRGSGSGGSGGSGS | | |
| | GKLPTDPLSLQELRREFTVSLYL | | |
| | ARKLLSEVQGYVHSFAESRLPGV | | |
| | NLDLLPLGYHLPNVSLTFQAWH | | |
| | HLSDSERLCFLATTLRPFPAMLG | | |
| | GLGTQGTWTSSEREQLWAMRLD | | |
| | LRDLHRHLRFQVLAAGFKCSKE | | |
| | EEDKEEEEEEEEEEKKLPLGALG | | |
| | GPNQVSSQVSWPQLLYTYQLLH | | |
| | SLELVLSRAVRDLLLLSLPRRPGS | | |
| | AWDS | | |
| | L. lactis usp45 secretion | | |
| | signal mouse EBI3 aa 18-228 | | |
| | Linker Mouse p28 aa 30-234 ) | | |
| Inflammatory | IL-10 | Cell lysate fraction | Benbouziane, B., Ribelles, |
| bowel disease | | was reported to | P., Aubry, C., Martin, R., |
| | | contain 40.72 ng/mL | Kharrat, P., Riazi, A., . . . & |
| | | of IL-10. | Bermúdez-Humaran, L. G. |
| | | | (2013). Development of a |
| | | | Stress-Inducible Controlled |
| | | | Expression (SICE) system |
| | | | in Lactococcus lactis for |
| | | | the production and delivery |
| | | | of therapeutic molecules at |
| | | | mucosal surfaces. Journal |
| | | | of Biotechnology, 168(2), |
| | | | 120-129. |
| H. pylori | artilysin (endolysin/holin | Minimal inhibitory | Xu, D., Zhao, S., Dou, J., |
| infection | fusion protein) | concentration was | Xu, X., Zhi, Y., & Wen, L. |
| | | reported to be 100 - | (2021). Engineered |
| | | 500 ug/mL | endolysin-based |
| | | | “artilysins” for controlling |
| | | | the gram-negative |
| | | | pathogen Helicobacter |
| | | | pylori. AMB Express, |
| | | | 11(1), 1-9. |
| Colitis | (R)-3 -hydroxybutyrate | 1.2 uM was the | Yan, X., Liu, X. Y., Zhang, |
| | | reported (in vivo) | D., Zhang, Y. D., Li, Z. H., |
| | | dosage of 3HB with | Liu, X., & Chen, G. Q. |
| | | an initial seeding of | (2021). Construction of a |
| | | 5 × 10^10 cells | sustainable 3- |
| | | | hydroxybutyrate-producing |
| | | | probiotic Escherichia coli |
| | | | for treatment of colitis. |
| | | | Cellular & Molecular |
| | | | Immunology, 18(10), |
| | | | 2344-2357. |

The disclosure also provides for methods of inducing electrostimulative release of a signaling molecule or therapeutic protein from a bacterial cell comprising: (a) growing one or more bacterial cells of a first population of cells in the systems disclosed herein; (b) and introducing one or more stimuli to the one or more bacterial cells. In some embodiments, the stimuli is performed through creating a voltage drop at an electrode positioned proximate to or within the vessel or well comprising the disclosed bacterial population or bacterial cell populations. In some embodiments, the one or more stimuli is a readout of quorum sensing, wherein, the presence of a receptor density activate a promoter operably linked to a protein that disrupts a cell wall of the bacteria. In some embodiments, the protein that disrupts the cell wall is TolAIII or functional fragment thereof.

In some embodiments, the methods of treatment or methods of manufacturing a recombinant protein comprise a step of electrostimulating the bacterial cell or bacterial cell population within the device by way of an electrode that from about 0.1 to about 1.5 volts of electricity. In some embodiments, the methods of treatment comprise a step of electrostimulating the device with from about 0.5 to about 1.5 volts of electricity. In some embodiments, the methods of treatment comprise a step of electrostimulating the device with from about 0.5 to about 2.5 volts of electricity. In some embodiments, the methods of treatment comprise a step of electrostimulating the device with from about 0.3 to about 2 volts of electricity. In some embodiments, the methods of treatment comprise a step of electrostimulating the device with from about 0.5 to about 3.5 volts of electricity. In some embodiments, the methods of treatment comprise a step of electrostimulating the device with from about 0.5 to about 4 volts of electricity. In some embodiments, the methods of treatment comprise a step of electrostimulating the device with from about 0.5 to about 4.5 volts of electricity. In some embodiments, the methods of treatment comprise a step of electrostimulating the device with from about 0.5 to about 5 volts of electricity. In some embodiments, the methods of treatment comprise a step of electrostimulating the device with from about 0.5 to about 5.5 volts of electricity. In some embodiments, the methods of treatment comprise a step of electrostimulating the device with from about 0.5 to about 6 volts of electricity. In some embodiments, the methods of treatment comprise a step of electrostimulating the device with from about 0.5 to about 6.5 volts of electricity. In some embodiments, the methods of treatment comprise a step of electrostimulating the device with from about 0.5 to about 7 volts of electricity. In some embodiments, the methods of treatment comprise a step of electrostimulating the device with from about 0.5 to about 7.5 volts of electricity. In some embodiments, the methods of treatment comprise a step of electrostimulating the device with from about 0.5 to about 8 volts of electricity. In some embodiments, the methods of treatment comprise a step of electrostimulating the device with from about 0.5 to about 8.5 volts of electricity. In some embodiments, the methods of treatment comprise a step of electrostimulating the device with from about 0.5 to about 9 volts of electricity. In some embodiments, the methods of treatment comprise a step of electrostimulating the device with from about 0.5 to about 9.5 volts of electricity. In some embodiments, the methods of treatment comprise a step of electrostimulating the device with from about 0.5 to about 10 volts of electricity. In some embodiments, the methods of treatment comprise a step of electrostimulating the device with from about 1.0 to about 1.5 volts of electricity. In some embodiments, the methods of treatment comprise a step of electrostimulating the device with from about 1.0 to about 2 volts of electricity. In some embodiments, the methods of treatment comprise a step of electrostimulating the device with from about 1.0 to about 2.5 volts of electricity. In some embodiments, the methods of treatment comprise a step of electrostimulating the device with from about 1.0 to about 3 volts of electricity. In some embodiments, the methods of treatment comprise a step of electrostimulating the device with from about 1.0 to about 3.5 volts of electricity. In some embodiments, the methods of treatment comprise a step of electrostimulating the device with from about 1.0 to about 4 volts of electricity. In some embodiments, the methods of treatment comprise a step of electrostimulating the device with from about 0.5 to about 4.5 volts of electricity. In some embodiments, the methods of treatment comprise a step of electrostimulating the device with from about 1.0 to about 5 volts of electricity. In some embodiments, the methods of treatment comprise a step of electrostimulating the device with from about 1.0 to about 5.5 volts of electricity. In some embodiments, the methods of treatment comprise a step of electrostimulating the device with from about 1.0 to about 6 volts of electricity. In some embodiments, the methods of treatment comprise a step of electrostimulating the device with from about 1.0 to about 6.5 volts of electricity. In some embodiments, the methods of treatment comprise a step of electrostimulating the device with from about 0.5 to about 7 volts of electricity. In some embodiments, the methods of treatment comprise a step of electrostimulating the device with from about 1.0 to about 7.5 volts of electricity. In some embodiments, the methods of treatment comprise a step of electrostimulating the device with from about 1.0 to about 8 volts of electricity. In some embodiments, the methods of treatment comprise a step of electrostimulating the device with from about 1.0 to about 8.5 volts of electricity. In some embodiments, the methods of treatment comprise a step of electrostimulating the device with from about 1.0 to about 9 volts of electricity. In some embodiments, the methods of treatment comprise a step of electrostimulating the device with from about 1.0 to about 9.5 volts of electricity. In some embodiments, the methods of treatment comprise a step of electrostimulating the device with from about 0.1 to about 10 volts of electricity. In some embodiments, this electrostimulating step is performed in a microenvironment free of an electron redox mediator. In some embodiments, the redox mediator any cyano-derived electron mediator, such as pyocyanin or ferricyanide.

The disclosure further relates to a method of inducing release or burst of therapeutic molecule or signaling molecule comprising: (i) exposing the one or plurality of bacterial cell populations to a first stimulus (such as an electric charge) that stimulates expression of a nucleic acid sequence encoding a therapeutic molecule; and subsequently (ii) exposing the one or plurality of bacterial cell populations to a second stimulus (such as a quorum of number or density of bacterial cells) to activate expression of at least a domain of an exogenously regulated transmembrane protein, such as TolAIII, that creates pores within the cellular membrane of the one or plurality of bacterial cells. In some embodiments, the bacterial cell population or bacterial cell comprises a nucleic acid sequence that encodes a pore-forming protein or variant thereof. In some embodiments, the pore-forming protein is a Pore-forming toxin (PFT) identified in Rosado, et. al., Cell Microbiol. 2008 September; 10(9): 1765-1774, and incorporated by reference in its entirety. In some embodiments the bacterial cell expresses a nucleic acid sequence encoding a bacterial transmembrane protein domain, such as TolAIII, operably linked to a quorum sensing regulatory sequence or promoter such as SEQ ID NO:13 or a variant thereof that comprises at least about 70% sequence identity to SEQ ID NO:13. In some embodiments, the bacterial cell population comprises a nucleic acid sequence comprising at least about 70% sequence identity to SEQ ID NO: 19 or a variant or functional fragment thereof operably linked to a quorum sensing regulatory sequence or promoter such as SEQ ID NO:13 or a variant thereof that comprises at least about 70% sequence identity to SEQ ID NO:13. In some embodiments, the bacterial cell population comprises: (i) a nucleic acid sequence comprising at least about 70% sequence identity to SEQ ID NO: 19 or a variant or functional fragment thereof operably linked to a quorum sensing regulatory sequence or promoter such as SEQ ID NO:13 or a variant thereof that comprises at least about 70% sequence identity to SEQ ID NO:13; and (ii) a nucleic acid sequence encoding a therapeutic molecule or signaling molecule (at least about 70% sequence identity to a disclosed therapeutic molecule or a variant or functional fragment thereof) operably linked to a constitutive or inducible promoter such as SEQ ID NO:1 or a variant thereof that comprises at least about 70% sequence identity to SEQ ID NO:1. Under correct bacterial cell number or density, a bacterial cell or plurality of bacterial cells expressing the therapeutic molecule or signaling molecule constitutively or periodically can release the recombinantly made molecule after expression of SEQ ID NO:19 or a variant or functional fragment thereof.

Some embodiments include a method of isolating a protein or manufacturing a recombinant protein. The method can comprise providing a bioreactor operable to vitally support one or more disclosed microorganisms. Further, providing the bioreactor can comprise: providing one or more bioreactor walls, the bioreactor wall(s) comprising at least one bioreactor wall material and the at least one bioreactor wall material being flexible; and coupling together the bioreactor wall(s) so that the bioreactor wall(s) at least partially form a bioreactor cavity configured to contain the microorganism(s) and a fluidic support medium. In these or other embodiments, at least one of (a) when the microorganism(s) are E. coli, the bioreactor can vitally support the microorganism(s) such that an average therapeutic molecule or signaling molecule produce from about 0.01 to about 0.1 mg of protein per 24-hour cycle. In these or other embodiments, coupling together the bioreactor wall(s) so that the bioreactor wall(s) at least partially form the bioreactor cavity can comprise bonding together by heat welding the bioreactor wall(s) so that the bioreactor wall(s) at least partially form the bioreactor cavity. Further, the at least one bioreactor wall material can comprise a polymer material.

The method can comprise a step of: inoculating a bioreactor with one or more first microorganisms and a first fluidic support medium, the bioreactor comprising one or more bioreactor walls at least partially forming a bioreactor cavity, the bioreactor being configured to be at least one of folded up or rolled up, the bioreactor wall(s) comprising at least one bioreactor wall material, and the at least one bioreactor wall material being flexible; and vitally supporting the first microorganism(s) with the bioreactor such that the bioreactor produces about 1.0, 1.1, 1.2, 1.3, 1.4, 1.5, 1.6, 1.7, 1.8, 1.9 mg of protein per milliliter of fluid in the vessel or bioreactor cavity per day.

The following examples are meant to be non-limiting examples of how to make and use the embodiments disclosed in this application. Any publications disclosed in the examples or the body of the specification are incorporated by reference in their entireties, except for any statements contradictory to the express disclosure herein, subject matter disclaimers or disavowals, and except to the extent that the incorporated material is inconsistent with the express disclosure herein, in which case the language in this disclosure controls. Incorporation by reference of the following shall not be considered an admission by the applicant that the incorporated materials are prior art to the present disclosure, nor shall any document be considered material to patentability of the present disclosure.

### EXAMPLES

### Example 1

### 1. Bioelectronic Signal Transduction Through OxyR-Based Electrogenetics

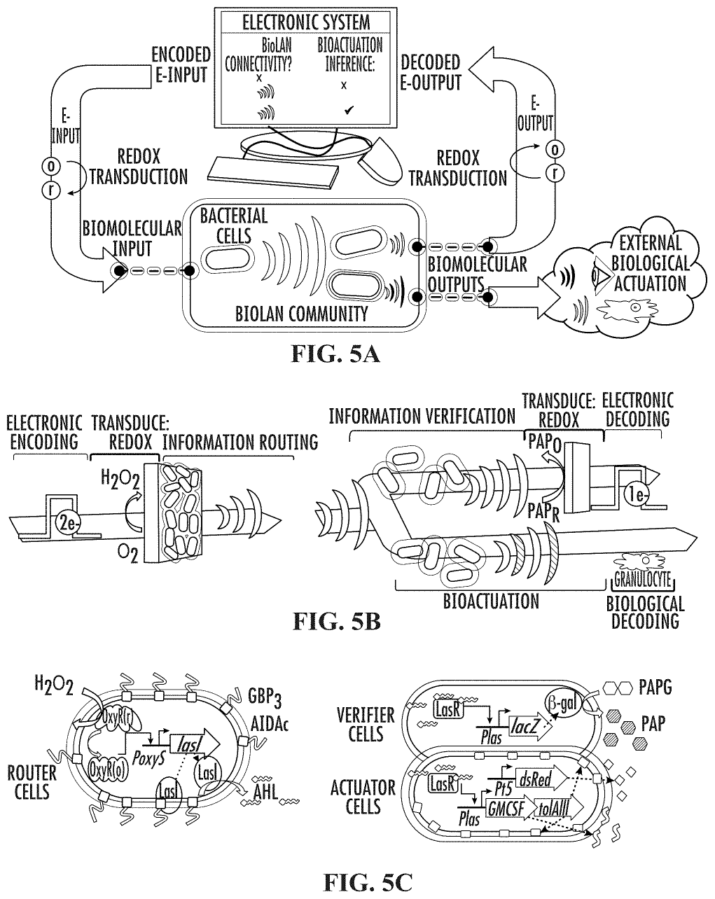

In the present work, electrode-generated redox signals, without mediators and with surface-engineered bacteria, initiate transduction and, when coupled with a second, recognition-based redox event, propagate and validate information flow between the electronic system and a community of engineered microbial cells (denoted BioLAN, FIG. 5A), is created. We use the principles of synthetic biology to coopt native redox signaling for modular circuitry that coordinates information processing across populations.

The electrochemically “plugged-in” BioLAN serves as an embedded, biological local area network that transmits electronic information. The BioLAN's community nature provides several advantages—signal propagation, spatial organization, and division of labor across populations^{16, 17}. Each member is engineered for specific signal processing tasks—signal Routing, Verification, and bio-Actuation (FIGS. 5A-5C). Initially, electronic input is redox-gated for electronic-to-bio signal transduction. The electrochemical reduction of molecular oxygen to hydrogen peroxide (henceforth, ‘peroxide’) is paired with a peroxide-inducible genetic circuit derived from the OxyR oxidative stress response regulon¹⁸. Once repurposed as an electrogenetic gate, cells are able to translate redox input into an orthogonal molecular cue (acyl homoserine lactone (AHL) from Pseudomonas aeruginosa) for information routing (FIG. 5B). Importantly, these Routing cells are “hardwired” to the active electrode via engineered surface adhesion. This enables direct, uniform interception of the peroxide signal and its transmission as AHL to the remaining BioLAN constituents.

The BioLAN includes two mobile populations of AHL recipients that interpret and relay signals downstream; thus a signal pathway from “encoded” electronic input to multiplexed genetic activation is established via AHL (FIG. 5C). Verifier cells confirm the relayed information providing an electronically-measured response through expression of β-galactosidase (β-gal) and its cleavage of 4-aminophenyl-β-D-galactopyranoside (PAPG) to the electronically-detected p-aminophenol (PAP). Bioactuator cells are also induced by AHL, directing the synthesis and secretion of a hard-to-detect therapeutic, granulocyte-macrophage colony stimulating factor (GMCSF)²¹. Importantly, the electronic output from Verifier cells serves to indicate (i) successful signal propagation and information transfer into the BioLAN, and, because their signal-receiving genetic circuitry is identical to the Actuator cells, (ii) the synthesis of GMCSF. Thus, BioLAN signaling both commands and confirms putative delivery of the therapeutic. That is, bidirectional (i.e., electronic to bio to electronic) communication across a bioelectronics interface can be established by taking advantage of the redox-specific biological processing of electrochemically-encoded and decoded information. The resulting bioelectronics platform, including an electronically “plugged-in” microbial BioLAN, fulfills the fundamental requirements for implementing remote biological functions within networked biohybrid devices.

To encode and transduce electronic information for cells, we developed an electrogenetic system using the native transcriptional activator, OxyR. E. coli rapidly uptakes and enzymatically degrades peroxide upon exposure, and its transient intracellular presence oxidizes OxyR (OxyR(o))^{22, 23}. This can elicit a strong native regulatory response, including upregulation of oxyS from the PoxyS promoter^{24, 25}. For electrogenetic system development, peroxide can be electrochemically generated from oxygen under physiological conditions²⁶.

### Methods

Chemicals. 4-Aminophenyl β-d-galactopyranoside (PAPG), 4-aminophenol (PAP), and ortho-Nitrophenyl-β-galactoside (ONPG), propidium iodide (PI) were from Sigma-Aldrich. PAPG was dissolved in deioinized water and PAP and ONPG were dissolved in 0.1 M phosphate buffer (PB). Agar, KCl, LB broth (Miller), M9 salts, glucose, glycerol, casamino acids, magnesium sulfate (MgSO₄), calcium chloride (CaCl₂), 3-(N-morpholino) propane sulfonic acid (MOPS), and hydrogen peroxide (H₂O₂, 30%) were from Sigma Aldrich. N-3-oxo-dodecanoyl-L-Homoserine lactone (AHL) from Cayman Chemicals. Biological fixatives included formaldehyde (ThermoFisher Scientific) and paraformaldehyde (Electron Microscopy Sciences).

Cell culture and media. Unless otherwise indicated, cells were grown overnight in LB at 37° C., 250 rpm shaking, inoculated at 1-2% in LB or M9 media, and grown until the indicated cell density (OD600). M9 media consisted of 1×M9 salts, 0.4% glucose, 0.2% casamino acids, 2 mM MgSO₄, 0.1 mM CaCl₂ and, optionally, 100 mM MOPS.

Molecular biology and genetic engineering. All enzymes, competent cells, and reagents were from New England Biolabs (NEB), used according to provided protocols. Polymerase chain reactions (PCR) used Q5 polymerase and primers in Table 4 (gene sequences in Table 3). DpnI digestion, polynucleotide kinase phosphorylation, T4 ligations, Gibson assembly, and E. coli chemical transformation were performed using NEB products protocols. DNA clean-up, gel extraction, and plasmid prep kits were from Zymo Research and used provided protocols. Synthetic gene fragments GBP3 and proD were from Integrated DNA Technologies (IDT). Final plasmid constructs were sequence-verified. Constructs included pOxyRS-sfGFP-laa, pOxyRS-LacZ-laa, pOxyRS-LacZ, pOxyRS-LasI-laa, pOxyRS-LasI, pBla-GBP₃, pBla-His6-AIDA, pBla-Linker-AIDA, pAHL-LacZ, and pAHL-GMCSF.

Binding of GBP₃⁺ cells on chip. Cells were grown overnight in LB medium, diluted in fresh medium, and grown until mid-log OD (unless otherwise indicated). Electrodes or pre-cut gold chips were cleaned sequentially in acetone, isopropanol, and water solutions, then dried with a nitrogen gas stream, and treated with ozone for 1 h using a PSD Series UV ozone system (Novascan Technologies, Boone, Iowa). The immobilization protocol was customized to each strain for at least 40% surface coverage. Small volumes (50-100 μL) of cultures were either applied directly to the surfaces of face-up gold chips to achieve edge-to-edge surface coverage, or alternatively, first centrifuged (8000 rpm, 3 min) for 4-fold concentration prior to application. After a 30 minute RT static incubation, the cells were washed in PBS face up (each chip placed inside a well of a 6-well culture plate) for 15-30 minutes at 150 rpm. Cell-covered electrode chips were incubated in PBS at RT until next steps.

Fluorescent cell staining. To visualize electrode-immobilized bacteria, cells were fluorescently stained using a Live/Dead Baclight Bacterial Viability Kit (Molecular Probes). Syto9 and propidium iodide (PI), were used alone or in combination. In each case, the reagents were diluted between 667 and 1000-fold in PBS, then applied to the cell-covered surfaces and incubated in the dark for 45 min, undisturbed, to stain immobilized cells. The, chips were the rinsed and stored in PBS. Initial characterization of surface display protein sequence (Linker-AIDA, His-AIDA, or GBP3-AIDA protein) contribution to bacterial immobilization was determined by live cell imaging, where only the Syto9 stain was applied. For subsequent analyses of immobilized cell density, a fixative solution (prepared at 2%) was first applied for 30 min to preserve the samples, followed by a PBS rinse and application of a PI solution prior to imaging. To quantify viability of cells bound to electrode chips, Syto9 and PI were applied in combination, followed by the fixative solution, with PBS rinsing in between steps. In this case, both green and red fluorescence images were captured. To calculate percent of dead cells, the total cell area of PI-stained cells in the image was divided by the PI+Syto-9 stained cell area. The fluorescence images were converted to 8-bit, consistently thresholded, and the cell areas were calculated using ImageJ (NIH). For dead-only staining of planktonic cultures, PI was used at 0.5 mg/ml in 0.85% NaCl. After 0.5-2 mL of the cell culture (depending on cell density) was pelleted, 50 μl of the PI solution was added, cells were re-suspended, and incubated in the dark for 30 minutes. After a PBS rinse, cell fluorescence was read on the flow cytometer or imaged by the fluorescence microscope.

Fluorescence microscopy. An Olympus BX53 microscope with the 49002-BX3; ET-EGFP/FITC/CY2 470/40X, BS495, 525/50M filter cube was used to visualize sfGFP-producing and cells stained with Syto9 fluorescent dye. For PI-stained cells the 49008-BX3; ET-MCHERRY/TXRED 560/40X BS585, 630/75M filter cube was used. Additional imaging utilized either an Olumpus BX60 for green fluorescence or a Zeiss Axio Observer 7 (Carl Zeiss AG, Oberkochen, Germany) with Colibri LED illumination and a Filter Set 43 HE Cy3 for red fluorescence. Composite fluorescence images were obtained using a Zeiss LSM700 confocal microscope.

Peroxide electrochemical setup and generation. For electrochemical peroxide generation a gold-patterned silicon wafer chip (with electrode dimensions 1 cm², unless otherwise indicated) was used as a working electrode. A coiled platinum wire (BaSi) with surface area larger than that of the working electrode was used as the counter electrode. An Ag/AgCl (BaSi) reference electrode was used. Using sufficient volumes of the indicated solution or media, the electrodes were completely submerged, with the working and reference electrodes in a separate glass vial than the counter electrode, connected by two salt bridges. The working electrode solution was undisturbed (or, where indicated, stirred with a 7 mm stir bar and a mini magnetic stirrer). The electrodes were connected to a potentiostat (either 700-series CH Instruments or BioLogic VMP3). Chronoamperometry, poised at −0.4V for the indicated duration, was performed to generate hydrogen peroxide. The endpoint charge was recorded for each run.

Agar salt bridges consisted of 6 inch-long 1.2 mm OD, 0.9 mm ID glass capillary tubes bent into a U shape after brief heating under a Bunsen burner. A 3% agar solution with 1 M potassium chloride (KCl) was heated and added into the bent capillary tube. Tubes were cooled by immersion in a 3 M KCl solution and stored in 3 M KCl at 4° C.

Hydrogen peroxide determination. The Pierce Quantitative Peroxide Assay Kit (Aqueous) (ThermoFisher Scientific) was used to quantify peroxide according to manufacturer's instructions. Briefly, the working reagent (WR) was prepared by mixing 1 volume of Reagent A with 100 volumes of Reagent B, with at least 200 μl prepared for each sample to be assayed. 10 volumes of the WR were added to 1 volume of sample (typically 200 μl WR to 20 μl sample) into a well of a clear bottomed 96 well plate. The reaction was mixed and incubated for 15-20 minutes, after which a Spectramax M3 plate reader was used to measure the absorbance at 595 nm. Sample peroxide concentration was calculated by comparison to a standard curve (dilutions of 30% peroxide) performed the same day.

Quantification of electrochemical peroxide generation rate. Using experimentally-obtained data for peroxide concentration and charge, a characteristic linear relationship was established for both stirring and non-stirring conditions (FIGS. 15A-15E). The efficiency of oxygen reduction to peroxide by the supplied charge was determined by comparing the actual peroxide level to the theoretical yield. The charge was converted to moles of electrons using Faraday's constant (96485 C mol⁻¹) and accounting for two electrons required to produce one hydrogen peroxide molecule. The oxygen reduction efficiency was found to be constant at 0.59 regardless of stirring conditions (FIG. 15F).

Quantification of cell peroxide consumption rates. Cells were grown overnight as above. To quantify consumption of peroxide, T7Express E. coli with the pOxyRS-LacZ-laa plasmid were diluted to optical densities (measured at 600 nm) of 0.025, 0.05, and 0.1, each with 100 μM peroxide in 3 mL of LB medium in 15 mL culture tubes in a 37° C. incubator at 250 rpm. 20 μl aliquots were assayed at each timepoint, and peroxide levels were compared to a standard curve using the Pierce Quantification Peroxide Assay Kit.

For quantification of peroxide consumption from electrode-immobilized cells, the bacteria were first assembled onto gold-coated wafer chips (150 mm²) as described in Methods. After rinsing superfluous bacteria from the chips, each was submerged in 2 mL M9 medium supplemented with antibiotics and 100 μM peroxide. The negative control was a sterile gold chip in the solution and the positive control contained suspended cells at an optical density (600 nm) of 0.025. All samples were incubated statically at 37° C. for 2 h, during which 20 μl aliquots were assayed for peroxide concentration at regular timepoints. At the end of the timecourse, the cell-immobilized chips were fixed with paraformaldehyde, followed by PI staining. Image analysis was used to quantify the exact electrode dimensions based on photographs and on-chip cell densities based on fluorescence imaging of the chip surfaces. The rate of peroxide accumulation for each sample was determined by normalizing the assay-measured number of peroxide molecules in solution to cell number.

Measurement of growth effects of peroxide. To quantify cell growth in the presence of peroxide, E. coli T7Express cells with the pOxyRS-sfGFP-laa and pBla-GBP₃-AIDA plasmids were cultured overnight as described above. The cells were diluted in the M9 media with antibiotics to an OD600 of 0.025. Either solution-based or electrochemically-generated peroxide was added to the cells at different concentrations, as indicated, in triplicates. A Bioscreen C machine (Growth Curves USA) was set up at 37° C., high shaking, with 400 μl per well, and recorded OD600 measurements every 15 minutes, using a Growth Rates program to calculate lag and doubling time.

Colony Forming Unit (CFU) determination. Treated or untreated cells were diluted at least 1000 times, and 100 μL of these dilutions were plated on LB+1.5% agar plates with the appropriate antibiotics. After an overnight incubation at 37° C., colonies were counted and CFU's were calculated.

Flow cytometry. Flow cytometry was used to quantify peroxide-induced sfGFP fluorescence intensity, live/dead cell ratios, and quantum dot labeling of cell surfaces. A BD Accuri C6 with an autosampler was used to measure sfGFP and live/dead cell numbers. Reported values are the mean fluorescence values in the FL-1 channel (green) or FL-3 (red). Quantum dot-labeling of cells was determined using a BD Canto II by first establishing a threshold for unlabeled cells using the 530/30 filter and then comparing above-threshold counts between GBP3− and + strains once mixed with quantum dots. All data is supported by at least 20,000 events and consistently gated across samples.

Miller assay. The Miller assay was performed at measurement timepoints according to standard protocols. Briefly, cells were lysed with chloroform and sodium dodecyl sulfate (SDS) to release β-galactosidase. The substrate ONPG was added and cleaved by β-galactosidase into a yellow molecule, o-nitrophenol. Absorbance of sample sets at 600, 550, and 420 nm was quantified by either a Spectramax M3 or BioTek Synergy plate reader. Absorbance at 600 nm was measured from 250 μL of culture sample prior to assay preparation and the absorbance at 420 and 550 nm were measured from 200 μL of the sample after assaying for β-galactosidase activity. Miller Units were calculated as per the standard protocol.

Electrochemical PAP measurement. PAP was detected electrochemically through cyclic voltammetry (CV), chronoamperometry (CA), and differential pulse voltammetry (DPV). All measurements were performed with a CHI Instruments 700-series (CH instruments, Inc.) or VMP3 (BioLogic Science Instruments) using gold working electrode (2 or 3 mm diameter, CH Instruments, Inc.), a coiled platinum counter electrode with working area larger than that of the working electrode (BASi), and an Ag/AgCl reference electrode (BASi). CVs were run from −0.15 to 0.3 V at a scan rate of 50 mV/s. The current at the reduction peak was used to measure PAP concentration. DPV was run from −0.16 to 0.36 V, at 2 mV step increments, with a 50 mV amplitude, 0.5 s pulse width, and a 0.5 s pulse period. The DPV peak height was calculated either automatically or manually for calibration to PAP concentration.

General and co-culture electro-induction setup. The same electrochemical setup used for peroxide generation was used for electro-induction, and was placed inside of a mini-incubator set at 37° C. Cells were added to a final OD600 of 0.025 in the working electrode vial, or were pre-assembled onto the working electrode. M9 medium with the appropriate antibiotics was used. Peroxide was generated via chronoamperometry as indicated above, with voltage application for a specified duration (eg 300 s). For planktonic cells, the entire volume was then pipetted into a culture tube, and placed in a 37° C. shaking incubator at 250 rpm, from which samples were removed at indicated time intervals. For electrode-assembled cells, the working electrode was placed in a culture tube with fresh media for shaking incubation at 37° C. and 250 rpm. In experiments that electrochemically probed β-galactosidase induction, PAPG was added after electro-induction and prior to incubation at 5 mg/mL. For co-cultures, The Router cells were inoculated to an OD600 of 0.025 in the working electrode solution for planktonic co-cultures or pre-assembled directly on the working electrode, which was submerged in the co-culture. In double-culture experiments, the Verifier cells were co-inoculated to a final OD600 of 0.1. In triple-culture experiments, Verifier and Actuator cells were inoculated to a final OD600 of 0.075 and 0.025, respectively (FIG. 10B).

AHL quantification. AHL quantification was performed by bioluminescence assay. AHL reporter cells JLD271 pAL105 were grown overnight in LB at 37° C. shaker and 250 rpm with the appropriate antibiotics. The following day, standard AHL solutions (concentration range) were prepared in LB. The reporter cells were diluted 2500× in LB with the appropriate antibiotics. To each, 90 μL of diluted reporter cells and 10 μl of the standard dilutions were mixed in a 5 mL culture tube and prepared in duplicate. Experimental conditioned media samples were prepared similarly after sterile-filtering and diluting between 5 and 10× to maintain a linear assay range. Culture tubes with reporter cells and conditioned media samples were incubated at 30° C. and 250 rpm shaking for 3 hours. Luminescence was measured with a GloMax®-Multi Jr (Promega, Madison, Wis., USA). AHL concentration of each sample was calculated using the standard curve.

Electrode chip fabrication. Gold electrodes were prepared by cutting gold-coated silicon wafers (Ted Pella) or purchased (Platypus Technologies). Gold coating was 50-100 nm in thickness with a 5 nm sublayer of chromium or titanium. Alternatively, gold electrode arrays were patterned onto silicon wafers. First, metal deposition was performed on standard 4 inch silicon wafers using a Denton thermal evaporator (Denton Vacuum LLC, Moorestown, N.J., USA), with metal deposition rates at 2-3 Å/s. Specifically, a 50 nm chromium adhesion layer was evaporated, followed by 200 nm gold. Next, photolithography utilized direct writing of photoresist via a DWL66fs laser writer (Heidelberg Instruments, Heidelberg, Germany), guided by a laser exposure map designed in AutoCAD (Autodesk, San Rafael, Calif., USA). Photoresist spin-coating and development steps were performed using an EVG120 Automated resist processing system (EV Group, Sankt Florian am Inn, Austria). The patterned wafer was post-processed by etching, photoresist stripping, and cutting individual electrodes with a DAD dicing saw (DISCO, Tokyo, Japan).

Protein structure determination. Protein modeling and structural predictions were performed using Phyre2. This approach referenced the 4MEE crystal structure³ available through the RCSB Protein Data Bank (rcsb.org) in order to generate a homology model of GBP3-His-AIDAc protein used in this work.

Protein secretion analysis. The target proteins DsRedExpress2 and GMCSF were analyzed. The supernatants of sample aliquots were recovered after centrifugation (6000 g, 2 min) to separate bacteria and subdivided fluorescence analysis (stored chilled) or immunoassaying (stored frozen). The relative fluorescence intensity of samples was measured using a BioTek Synergy platereader to determine DsRedExpress2 levels; additional quantification to correlate fluorescence to protein concentration was performed using a Bioanalyzer 2100 (Agilent Technologies, Santa Clara, Calif.). The His-tagged GMCSF peptide was quantified using a His Tag ELISA Detection Kit (GenScript) according to the provided protocol and 5-10 fold dilutions of frozen samples.

Nanoparticle labeling of bacterial surfaces. For quantum dot labeling, CdSe/ZnS core-shell type quantum dots (Sigma Aldrich, carboxylic acid functionalized, fluorescence λₑₘ 520 nm) were diluted 100-fold into bacterial cultures (OD at 600 nm=1) resuspended in PBS at a 10× dilution. After incubating 1 h at room temperature, the cell cultures were rinsed twice prior to flow cytometry. For gold nanoparticle labeling, 20 nm gold nanoparticles (AuNPs, Ted Pella, #15705-20) were used to label the gold binding peptide constitutively expressed by E. coli cells. Transmission electron microscopy (TEM) was performed after labeling cells with Au NPs. Cells were diluted in a 2:1 mixture of PBS and water to an optical density at 600 nm of 0.28. The cells were then mixed with a thousand-fold excess of gold nanoparticles and incubated for one hour. The samples were prepared onto surface-treated copper grids covered with holey carbon films using a drop-casting method. The samples were viewed with a JEOL 2100F transmission electron microscope operated at 200 kV.

Model and simulations. The peroxide, promotor, and protein expression models were developed using first order reaction kinetics to describe OxyR kinetics and Hill functions to describe OxyR activation of gene expression. Models were implemented using Microsoft Excel when no spatial resolution was needed. Spatial dynamics were implemented in Matlab using the built-in PDESolver to solve the diffusion equation. Details are available in Table 5.

Implementation of modeled spatial dynamics. To understand the spatial dynamics of peroxide generation and consumption at the electrode with the cells either absent, present on the electrodes, or present in solution, the diffusion equation was solved in Matlab's built in PDESolver function. The box size was 3.5 mm per side with a one millimeter square electrode located in the center of one face of the box. The geometry was generated using the GeometryFromMesh function and 1 micron increments. Peroxide was generated at the electrode for 300 s and then stopped, reflecting a typical pulse increment. The overall simulation was run for 3600 s. Peroxide consumption was either absent (cell absence), occurring only on the electrode (immobilized cells), or in solution (peroxide pulse into a bulk culture). The details are provided in Table 5 and Table 6.

Cloning of peroxide-inducible genetic constructs. To construct a plasmid with a hydrogen peroxide-inducible circuit, the plasmid pOxyRS-sfgfp-laa was first engineered. First, the empty vector pBR322 was amplified with primers pBR322-1 and pBR322-2. The OxyR-pOxyS region was isolated from a boiled colony of E. coli MG1655 using OxyRS-1 and OxyRS-2. sfGFPlaa was isolated from pTGX using sfGFPlaa-1 and sfGFPlaa-2. After DpnI treatment, fragments were ligated by Gibson Assembly. This plasmid product, pOxy-sfGFPlaaV1, linearized with primers pOxyGFP-1 and pOxyGFP-2, and proD, amplified with primers proD-1 and proD-2, were ligated by Gibson Assembly to yield the final construct, pOxy-sfGFPlaa. Other genes of interest, lacZ and lasI, were substituted into the POxy construct, to create variants including and excluding the ssRA degradation tag (pOxy-LacZ, pOxy-LacZlaa, pOxy-LasI, and pOxy-LasIlaa). Here, pOxy-sfGFPlaa was linearized by PCR using PoxyAssem-R and PoxyAssem-F (to exclude the ssrA laa tag) or ssrA-F (to include the ssrA laa tag). Likewise, lacZ was amplified from pET-LacZ (Table 1) using PoxyLacZ-F and PoxyLacZ-R (for lacZ product) or LacZ-ssrA-R (for lacZlaa product); lasI was amplified from pLasI (Table 1) using PoxyLasI-F and PoxyLasI-R (for lasI product) or LasI-ssrA-R (for lasIlaa product). Each amplified gene was ligated into a corresponding pOxy or pOxy-laa backbone by Gibson Assembly to yield the final constructs and transformed into E. coli strain NEB10β (Table 2). Sequences of listed PCR primers are denoted in Table 4.

Cloning of surface display genetic constructs. To develop a plasmid construct for surface display of a gold-binding peptide, pGBP3 was engineered. First, the AIDA fusion gene with its preceding signal peptide was amplified from pET-Venus-AIDA (Table 1) using SigPep_pBla-ovhg-F and AIDA_pBla-ovhg-R primers. The host plasmid, pBla was linearized by PCR using pBla_5end-R and pBla_3end-F and the template pGB-FimH (Table 1) to open an insertion site at the bla promoter. The signal peptide-AIDA fragment was inserted into pBla by Gibson Assembly to achieve pBla-AIDA for constitutive AIDA expression under the bla promoter. Gbp₃ was purchased from IDT with a 5′ extension, TGCATTTGCAGTCGAC, preceding the ATG start codon. As such, GBP3assem-F and GBP3assem-R were used to amplify the gene. Additionally, the pBla-AIDA plasmid was linearized by PCR for GBP3 insertion downstream of the signal peptide using SurfDispIns-R and SurfDispIns-F; the two fragments were ligated by Gibson Assembly to yield pGBP3 for expression of the fusion gene: signal peptide-GBP3-linker-AIDA. Variations of pGBP3 were prepared as control surface display constructs. pHis was prepared by plasmid PCR using GSHis6-F and SurfDispIns-R; pLinker was prepared by plasmid PCR using KpnI-Linker-F and SurfDispIns-R. Each PCR was followed by phosphorylation, ligation, and transformation of the DNA product. Final plasmid constructs pGBP3, pHis, and pLinker were cotransformed with pET-LacZ or pOxy plasmids (pOxy-sfGFPlaa, pOxy-LacZlaa, pOxy-LasIlaa) in E. coli strains NEB10β, BL21(DE3), or T7 Express (Table 2). Sequences of listed PCR primers are denoted in Table 4.

Cloning of acyl homoserine lactone reporter genetic constructs. Acyl homoserine lactone responsive genetic constructs were developed using pAHL_reporter_Red_Green as a plasmid backbone (Table 1). To develop pAHL-LacZ, the sfGFP reporter gene was first replaced with a counterpart containing additional restriction endonuclease recognition sequences. Sfgfp was modified by PCR to include a SpeI site at the 5′ flank and both SacI and BamHI sites at the 3′ flank using primers sfGFP SpeI-F and sfGFP BamHISacI-R. Both the plasmid backbone and sfgfp insert were digested with SpeI and SacI and ligated to yield pAHL_reporter_Red_Green*. Next, LacZ was amplified using LacZ_SpeI-F and LacZ_BamHI-R, then inserted into the pAHL_reporter_Red_Green* backbone by digesting each component with SpeI and BamHI and ligation to yield pAHL-LacZ. Alternatively, the pAHL-GMCSF construct was prepared by Gibson Assembly. Assembled components included the GMCSF-TolAIII construct, amplified by PCR from pRM102 (Table 1) with pAHL-TolAIII-F and pAHL-TolAIII-R, and vector pAHL_reporter_Red_Green vector, linearized by PCR using pAHLassem-F and pAHLassem-R primers. pAHL-LacZ was transformed into an LW7 host and pAHL-GMCSF was transformed into a NEB10-β host (Table 2). Sequences of listed PCR primers are denoted in Table 4.

Model of hydrogen peroxide consumption by E. coli. Bacterial consumption of hydrogen peroxide was modeled as a pair of differential equations (Equations (S1-2)). As shown in FIG. 11B, under the conditions studied here, E. coli cells are the dominant consumer of hydrogen peroxide (H₂O₂) and, in the absence of bacteria, the peroxide concentration is nearly constant. Across the range of cell densities examined here, peroxide consumption by the cells can be modeled as a simple first order equation (Equation (S1)). Because of the time scale of the peroxide dissipation, it is also necessary to consider the continued growth and division of the bacteria (Equation (S2)). Rate constants k₁ and k_{double} are reported in Table 5. k₁ is obtained from fitting the peroxide concentration over time in FIG. 11B, using the approximation of 10⁹ cells per 1 OD600 unit. In FIG. 16C, the doubling time ranged from ca. 45 to 60 min and the rate constant (k_{double}) was taken from the higher end of this range.

$$\frac{d [ {H}_{2} {O}_{2} ]}{dt} = - {k}_{1} [ cells ] [ {H}_{2} {O}_{2} ] \qquad ( S1 )$$

$$\frac{d [ cells ]}{dt} = {k}_{double} [ cells ] \qquad ( S2 )$$

Model of hydrogen peroxide diffusion from an electrode. Matlab was used to simulate the spatial distribution of hydrogen peroxide during its electrochemical generation. The diffusion of hydrogen peroxide (H₂O₂) from the electrode was modeled with the diffusion equation (S3), taking 8.8×10⁻⁶ cm²·s⁻¹ as the diffusion constant of hydrogen peroxide in water.

$$\frac{\partial [ {H}_{2} {O}_{2} ]}{\partial t} = D {∇}^{2} [ {H}_{2} {O}_{2} ] \qquad ( S3 )$$

For the flux at the boundary, no hydrogen peroxide is generated or consumed except at the gold electrode surface. The flux of hydrogen peroxide generated at the electrodes using non-stirring conditions was taken from the measurements of hydrogen peroxide production in our system (FIG. 15A-15F), using the smallest electrode size implemented in this work (1 mm²) as the surface area unit; using a different electrode size would give very similar results. For conditions where cells are present in solution or at the electrode, an additional consumption term is included (Equation (S1)). Model parameters and their derivations from experiments are listed in Table 6. Model of OxyR-regulated protein expression. The behavior of the OxyR promoter was modeled as a set of first order reactions where OxyR is oxidized by peroxide and returns to the reduced state at a rate proportional to the amount of oxidized OxyR. The oxidized (OxyR(o)) and reduced (OxyR(r)) fractions sum to a constant value, normalized to 1 in this model. This results in a single differential equation to describe the OxyR oxidation state, Equation (S4). Rate constants are reported in FIGS. 15A-15F and are consistent with literature⁵³. Since the same promoter system regulates all protein expression systems modeled here, the parameters are kept constant across all protein models. OxyR(o) levels are shown in FIG. 11C with 10 min timepoints and 25 μM data shown in FIG. 6B and FIG. 6C, respectively.

$$\frac{d [ OxyR ( o ) ]}{dt} = - \frac{d [ Oxy R ( r ) ]}{dt} = {k}_{2} [ Oxy R ( r ) ] [ {H}_{2} {O}_{2} ] - {k}_{3} [ OxyR ( o ) ] \qquad ( S4 )$$

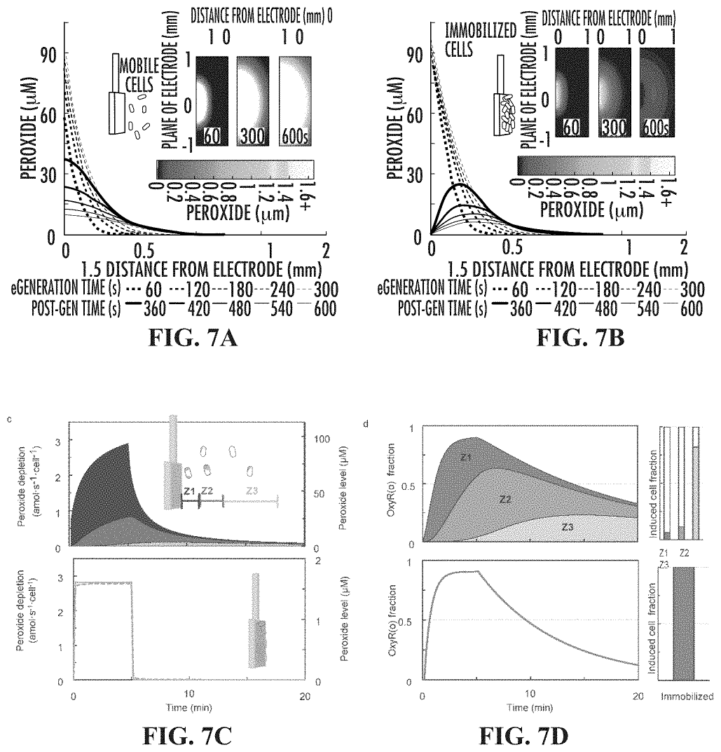

To model OxyR oxidation as a consequence of peroxide uptake during its electrochemical generation (FIGS. 7A-7B), the consumption rate of peroxide by the cells rather than the bulk peroxide concentration was used to calculate the rate of OxyR oxidation. This can be related to the bulk concentration model using Equation (S1), resulting in Equation (S5).

$$\frac{d [ OxyR ( o ) ]}{dt} = - \frac{d [ Oxy R ( r ) ]}{dt} = \frac{- {k}_{2} [ Oxy R ( r ) ] \frac{d [ {H}_{2} {O}_{2} ]}{dt}}{{k}_{1} [ cells ]} - {k}_{3} [ OxyR ( o ) ] \qquad ( S5 ) ]$$

The expression of three different proteins under the OxyR promoter was modeled (β-gal, sfGFP, and LasI). For the activity of the OxyR promoter itself, a Hill equation was used, modified by a proportionality constant to adjust for differences between the assays used to experimentally quantify the individual protein levels (k₇). The specific equations for the proteins are listed below, with the rate constants summarized in Table 5.

$$sfGFP : \frac{d [ GFP ]}{dt} = {k}_{7 \_ gfp} ( \frac{{k}_{3 gfp} [ O x yR ( o ) ]}{{K}_{3 gfp} + [ O x yR ( o ) ]} - {k}_{{6}_{gfp}} ) \qquad ( S6 )$$

$$β ‐ gal : \frac{d [ β gal ]}{dt} = {k}_{{7}_{-} gfp} ( \frac{{k}_{{3}_{β gal}} [ OxyR ( o ) ]}{{k}_{{3}_{β gal}} + [ OxyR ( o ) ]} + {k}_{{5}_{β g a l}} - {k}_{double} [ β gal ] ) \qquad ( S7 )$$

$$LasI : \frac{d [ LasI ]}{dt} = {k}_{{7}_{-} lasI} ( \frac{{k}_{3 \_ lasI} [ OxyR ( o ) ]}{{k}_{3 \_ lasI} + [ OxyR ( o ) ]} - {k}_{6 \_ lasI} ) \qquad ( S8 )$$

$$AHL : \frac{d [ AHL ]}{dt} = {k}_{7 \_ AHL} ( [ LasI ] + {k}_{{5}_{-} A H L} ) \qquad ( S9 )$$

The constants k₃ and K₃ were fit to the β-gal expression levels at different hydrogen peroxide amounts (FIGS. 12A-12F). For the β-gal, its ssrA degradation tag was ineffective in accelerating protein degradation, leading to an assumption of slow degradation compared to the timescale of the experiment. However, the protein is diluted as cell growth and division occurs, and this effect is included in the model (FIG. 12A). Additionally, there is a low level of leaky expression of the β-gal even without any addition of peroxide, and a parameter fit from the β-gal activity without peroxide is included to reflect this (k₅). Both the experimental data and modeled fits for dose-response between 0 and 100 μM peroxide and a timecourse of 180 min are plotted in FIG. 12B. The experimental data and resulting fit were each saturation-normalized (to the maximum value in Miller Units) and plotted in FIGS. 12B-12C.

For sfGFP and LasI, K₃ was taken from the β-gal expression since the promoter behavior should be similar across all three proteins; K_{3_GFP} was adjusted slightly. For protein degradation, sfGFP and LasI both have active ssrA degradation tags while that of β-gal is ineffective. A zero order reaction was used to represent the degradation kinetics expected at high protein concentrations, where the degradation machinery is expected to be saturated (FIG. 12A). This constant, k₆, was fit from the experimental AHL levels produced by LasI at 25 μM peroxide induction (FIG. 12E).

For sfGFP, the expected fluorescence intensities over time are displayed in arbitrary units that are equivalent to the median fluorescence intensities of experimental flow cytometry data (FIG. 12D and FIG. 13). Further, the expected fluorescent population fraction (% of total population) was calculated based on these trends using an error function. For a normally distributed function with mean (μ) and standard deviation (σ), the fraction of the function above a given threshold (τ) is given by Equation (S10).

$$Fr [ X > τ ] = 0. 5 + 0.5 erf ( \frac{{k}_{{8}_{GFP}} ( μ ) - τ}{σ \sqrt{2}} ) \qquad ( S10 )$$

The logarithm of fluorescence in experimental flow cytometry data was approximately normally distributed with a standard deviation of 0.35 in a base 10 logarithm (FIG. 13). τ was fit to the data to scale for both the overall fluorescence intensity and the measurement threshold. The μ values were taken from modeled sfGFP expression profiles (FIG. 12C) at 45 min, corresponding to the time where experimental data was also collected (FIG. 13). The experimental data and resulting fit were each saturation-normalized (to 100%) and plotted in FIG. 6B.

In the case of the LasI, the assay used in these experiments does not directly reflect the LasI concentration but rather the AHL produced by the LasI. This was modeled as a first order reaction in protein concentration assuming a large excess of the AHL precursor with the reaction constant k_{7_AHL} subsuming both the LasI concentration (arbitrarily scaled using k_{7_Last}) and its reaction rate; this constant is scaled LasI, based on the amount of AHL produced when 25 μM hydrogen peroxide is used to induce LasI expression (FIG. 12E) Additionally, baseline AHL is included as k_{5_AHL}, which is attributed to noise between basal AHL formation and bioluminescence in the detection assay (FIG. 12F). The experimental data and fits for LasI and AHL are reported in FIG. 12E; additionally, AHL data were normalized to the maximum concentration value and plotted in FIG. 6C.

TABLE 1
Plasmids used in this study.

| Plasmid name | Description | Source |
|---|---|---|
| pBR322 | | |
| pOxyRS-sfGFP-laa | pBR322 vector (Amp^{R}). OxyR | Adapted from |
| | transcriptional regulator constitutively | |
| | expressed by proD promoter. | |
| | sfGFP (with LAA ssRA tag) expression under | Rubens et al.²⁷ |
| | control of p_{oxyS} promoter. | |
| pOxyRS-LacZ-laa | Same as pOxyRS-sfgfp-laa but with the LacZ | This work |
| | protein instead of sfgfp. | |
| pOxyRS-LacZ | Same as pOxyRS-lacZ-laa, but without the | This work |
| | LAA ssRA tag. | |
| pOxyRS-LasI-laa | Same as pOxyRS-sfgfp-laa but with the lasI | This work |
| | protein instead of sfgfp. | |
| pOxyRS-LasI | Same as pOxyRS-laSi-laa, but without the | This work |
| | LAA ssRA tag. | |
| pGB-FimH | pACYC vector (Cm^{R}). Constitutive | Terrell et al.^{30, 54} |
| | expression of fimH under p_{bla}. | |
| pBla-Linker-AIDA | pACYC vector (Cm^{R}). Constitutive | This work |
| | expression under p_{bla} of AIDAc | |
| | with an N-terminal translocation sequence and | |
| | 54 amino acid linker (Terrell, Larrson et al). | |
| pBla-His6-AIDA | Same as pBlaLinker-AIDA with an N-terminal 6x | This work |
| | histidine tag. | |
| pBla-GBP3-AIDA | Same as pBlaHis6-AIDA with an N-terminal gold | This work |
| | binding peptide (3 repeats of MHGKTQATSGTIQS | |
| | amino acid sequence)³². | |
| pAHL_reporter_Red_Green | pET21a vector. Sfgfp production in response to | Rhoads et al.⁵⁵ |
| | AHL and constitutive dsRedExpress2. AmpR. | |
| pLasI | pET200 derivate, containing lasI under a | Stephens et al.⁴⁹ |
| | T7promoter, Km^{r} | |
| pAHL-LacZ | pAHL_reporter_Red_Green with lacZ replacing | This work |
| | the sfgfp. | |
| pAL105 | lasR⁺lasI::luxCDABE; Tet^{r} p15A origin | Lindsay et al.⁵⁶ |
| pRM102 | pET200 derivative, containing ompA-gmcsf- | McKay et al.²¹ |
| | cmyc-his6 and rbp-tolAIII expressed | |
| | polycistronically under the T7lac promoter, | |
| | Km^{r} | |
| pAHL-GMCSF | Same as pAHL-lacZ with genes for GMCSF and | This work |
| | TolAIII porin replacing lacZ, expressed in | |
| | tandem under pₗₐₛ. | |

TABLE 2
Cells used in this study.

| Cell name | Genotype | Source |
|---|---|---|
| NEB10β | Δ(ara-leu) 7697 araD139 fhuA ΔlacX74 | New England Biolabs |
| | galK16 galE15 e14- ϕ80dlacZΔM15 recA1 | |
| | relA1 endA1 nupG rpsL (Str^{R}) rph | |
| | spoT1 Δ(mrr-hsdRMS-mcrBC) | |
| LW7 | ZK126 ΔluxS::Kan | Wang et al.⁵⁷ |
| BL21 (DE3) | fhuA2 [lon] ompT gal (λ DE3) [dcm] | New England Biolabs |
| | ΔhsdS λ DE3 = λ sBamHIo ΔEcoRI-B | |
| | int::(lacI::PlacUV5::T7 gene1) i21 Δnin5 | |
| T7Express | fhuA2 lacZ::T7 gene1 [lon] ompT gal sulA11 | New England Biolabs |
| | R(mcr-73::miniTn10--Tet^{S})2 [dcm] | |
| | R(zgb-210::Tn10--Tet^{S}) endA1 | |
| | Δ(mcrC-mrr)114::IS10 | |
| JLD271 | E. coli K-12 ΔlacX74 sdiA271::Cam | Lindsay et al.⁵⁶ |

TABLE 3
Sequences of relevant genetic parts used in this study.

| Name | Sequence |
|---|---|
| SEQ ID NO. 1 | AAAGTTAAACAAAATTATTTGTAGAGGGAAACCGTTGTGGTCTCCCTGAATATA |
| proD promoter | TTATACGAGCCTTATGCATGCCCGTAAAGTTATCCAGCAACCACTCATAGACCT |
| | AGGGCAGCAGATAGGGACGACGTGGTGTTAGCTGTG |
| SEQ ID NO. 2 | TATCCATCCTCCATCGCCACGATAGTTCATGGCGATAGGTAGAATAGCAATGAA |
| PoxyS promoter | CGATTATCCCTATCAAGCATTCTGACTGATAATTGCTCACA |
| SEQ ID NO. 3 | CGAATTCATTAAAGAGGAGAAAGGTACC |
| Poxys Spacer + | |
| RBS | |
| SEQ ID NO. 4 | CGCGGAACCCCTATTTGTTTATTTTTCTAAATACATTCAAATATGTATCCGCTCA |
| B1a promoter + | TGAGACAATAACCCTGATAAATGCTTCAATAATATTGAAAAAGGAAGAGT |
| RBS | |
| SEQ ID NO. 5 | ATGAATAAGGCCTACAGTATCATTTGGAGCCACTCCAGACAGGCCTGGATTGTG |
| AIDAc | GCCTCAGAGTTAGCCAGAGGACATGGTTTTGTCCTTGCAAAAAATACACTGCTG |
| translocation | GTATTGGCGGTTGTTTCCACAATCGGAAATGCATTTGCA |
| signal peptide | |
| SEQ ID NO. 6 | ATGCACGGTAAGACACAAGCTACCAGTGGGACTATCCAGTCCATGCATGGAAA |
| GBP3-His6 | AACCCAAGCGACATCTGGAACGATTCAGAGCATGCATGGGAAAACTCAGGCAA |
| | CGTCGGGTACAATCCAATCA |
| SEQ ID NO. 7 | GTGAATAACAATGGAAGCATTGTCATTAATAACAGCATTATAAACGGGAATATT |
| AIDAc Linker | ACGAATGATGCTGACTTAAGTTTTGGTACAGCAAAGCTGCTCTCTGCTACAGTG |
| | AATGGTAGTCTTGTTAATAACAAAAATATCATTCTTAATCCTACAAAAGAAAGT |
| SEQ ID NO. 8 | ATAGGTAATACTCTTACCGTGTCAAATTATACTGGGACACCGGGAAGTGTTATT |
| AIDAc | TCTCTTGGTGGTGTGCTTGAAGGAGATAATTCACTTACGGACCGTCTGGTGGTG |
| | AAAGGTAATACCTCTGGTCAAAGTGACATCGTTTATGTCAATGAAGATGGCAGT |
| | GGTGGTCAGACGAGAGATGGTATTAATATTATTTCTGTAGAGGGAAATTCTGAT |
| | GCAGAATTCTCTCTGAAGAACCGCGTAGTTGCCGGAGCTTATGATTACACACTG |
| | CAGAAAGGAAACGAGAGTGGGACAGATAATAAGGGATGGTATTTAACCAGTCA |
| | TCTTCCCACATCTGATACCCGGCAATACAGACCGGAGAACGGAAGTTATGCTAC |
| | CAATATGGCACTGGCTAACTCACTGTTCCTCATGGATTTGAATGAGCGTAAGCA |
| | ATTCAGGGCCATGAGTGATAATACACAGCCTGAGTCTGCATCCGTGTGGATGAA |
| | GATCACTGGAGGAATAAGCTCTGGTAAGCTGAATGACGGGCAAAATAAAACAA |
| | CAACCAATCAGTTTATCAATCAGCTCGGGGGGGATATTTATAAATTCCATGCTG |
| | AACAACTGGGTGATTTTACCTTAGGGATTATGGGAGGATACGCGAATGCAAAA |
| | GGTAAAACGATAAATTACACGAGCAACAAAGCTGCCAGAAACACACTGGATGG |
| | TTATTCTGTCGGGGTATACGGTACGTGGTATCAGAATGGGGAAAATGCAACAGG |
| | GCTCTTTGCTGAAACTTGGATGCAATATAACTGGTTTAATGCATCAGTGAAAGG |
| | TGACGGACTGGAAGAAGAAAAATATAATCTGAATGGTTTAACCGCTTCTGCAGG |
| | TGGGGGATATAACCTGAATGTGCACACATGGACATCACCTGAAGGAATAACAG |
| | GTGAATTCTGGTTACAGCCTCATTTGCAGGCTGTCTGGATGGGGGTTACACCGG |
| | ATACACATCAGGAGGATAACGGAACGGTGGTGCAGGGAGCAGGGAAAAATAAT |
| | ATTCAGACAAAAGCAGGTATTCGTGCATCCTGGAAGGTGAAAAGCACCCTGGA |
| | TAAGGATACCGGGCGGAGGTTCCGTCCGTATATAGAGGCAAACTGGATCCATA |
| | ACACTCATGAATTTGGTGTTAAAATGAGTGATGACAGCCAGTTGTTGTCAGGTA |
| | GCCGAAATCAGGGAGAGATAAAGACAGGTATTGAAGGGGTGATTACTCAAAAC |
| | TTGTCAGTGAATGGCGGAGTCGCATATCAGGCAGGAGGTCACGGGAGCAATGC |
| | CATCTCCGGAGCACTGGGGATAAAATACAGCTTCTGA |
| SEQ ID NO. 9 | ATGAATATTCGTGATCTTGAGTACCTGGTGGCATTGGCTGAACACCGCCATTTTC |
| OxyR | GGCGTGCGGCAGATTCCTGCCACGTTAGCCAGCCGACGCTTAGCGGGCAAATTC |
| | GTAAGCTGGAAGATGAGCTGGGCGTGATGTTGCTGGAGCGGACCAGCCGTAAA |
| | GTGTTGTTCACCCAGGCGGGAATGCTGCTGGTGGATCAGGCGCGTACCGTGCTG |
| | CGTGAGGTGAAAGTCCTTAAAGAGATGGCAAGCCAGCAGGGCGAGACGATGTC |
| | CGGACCGCTGCACATTGGTTTGATTCCCACAGTTGGACCGTACCTGCTACCGCA |
| | TATTATCCCTATGCTGCACCAGACCTTTCCAAAGCTGGAAATGTATCTGCATGA |
| | AGCACAGACCCACCAGTTACTGGCGCAACTGGACAGCGGCAAACTCGATTGCG |
| | TGATCCTCGCGCTGGTGAAAGAGAGCGAAGCATTCATTGAAGTGCCGTTGTTTG |
| | ATGAGCCAATGTTGCTGGCTATCTATGAAGATCACCCGTGGGCGAACCGCGAAT |
| | GCGTACCGATGGCCGATCTGGCAGGGGAAAAACTGCTGATGCTGGAAGATGGT |
| | CACTGTTTGCGCGATCAGGCAATGGGTTTCTGTTTTGAAGCCGGGGCGGATGAA |
| | GATACACACTTCCGCGCGACCAGCCTGGAAACTCTGCGCAACATGGTGGCGGCA |
| | GGTAGCGGGATCACTTTACTGCCAGCGCTGGCTGTGCCGCCGGAGCGCAAACGC |
| | GATGGGGTTGTTTATCTGCCGTGCATTAAGCCGGAACCACGCCGCACTATTGGC |
| | CTGGTTTATCGTCCTGGCTCACCGCTGCGCAGCCGCTATGAGCAGCTGGCAGAG |
| | GCCATCCGCGCAAGAATGGATGGCCATTTCGATAAAGTTTTAAAACAGGCGGTT |
| | TAA |
| SEQ ID NO. 10 | ATGAGCAAAGGAGAAGAACTTTTCACTGGAGTTGTCCCAATTCTTGTTGAATTA |
| sfGFP | GATGGTGATGTTAATGGGCACAAATTTTCTGTCCGTGGAGAGGGTGAAGGTGAT |
| | GCTACAAACGGAAAACTCACCCTTAAATTTATTTGCACTACTGGAAAACTACCT |
| | GTTCCGTGGCCAACACTTGTCACTACTCTGACCTATGGTGTTCAATGCTTTTCCC |
| | GTTATCCGGATCACATGAAACGGCATGACTTTTTCAAGAGTGCCATGCCCGAAG |
| | GTTATGTACAGGAACGCACTATATCTTTCAAAGATGACGGGACCTACAAGACGC |
| | GTGCTGAAGTCAAGTTTGAAGGTGATACCCTTGTTAATCGTATCGAGTTAAAGG |
| | GTATTGATTTTAAAGAAGATGGAAACATTCTTGGACACAAACTCGAGTACAACT |
| | TTAACTCACACAATGTATACATCACGGCAGACAAACAAAAGAATGGAATCAAA |
| | GCTAACTTCAAAATTCGCCACAACGTTGAAGATGGTTCCGTTCAACTAGCAGAC |
| | CATTATCAACAAAATACTCCAATTGGCGATGGCCCTGTCCTTTTACCAGACAAC |
| | CATTACCTGTCGACACAATCTGTCCTTTCGAAAGATCCCAACGAAAAGCGTGAC |
| | CACATGGTCCTTCTTGAGTTTGTAACTGCTGCTGGGATTACACATGGCATGGATG |
| | AGCTCTACAAA |
| SEQ ID NO. 11 | ATGATCGTACAAATTGGTCGGCGCGAAGAGTTCGATAAAAAACTGCTGGGCGA |
| LasI | GATGCACAAGTTGCGTGCTCAAGTGTTCAAGGAGCGCAAAGGCTGGGACGTTA |
| | GTGTCATCGACGAGATGGAAATCGATGGTTATGACGCACTCAGTCCTTATTACA |
| | TGTTGATCCAGGAAGATACTCCTGAAGCCCAGGTTTTCGGTTGCTGGCGAATTC |
| | TCGATACCACTGGCCCCTACATGCTGAAGAACACCTTCCCGGAGCTTCTGCACG |
| | GCAAGGAAGCGCCTTGCTCGCCGCACATCTGGGAACTCAGCCGTTTCGCCATCA |
| | ACTCTGGACAGAAAGGCTCGCTGGGCTTTTCCGACTGTACGCTGGAGGCGATGC |
| | GCGCGCTGGCCCGCTACAGCCTGCAGAACGACATCCAGACGCTGGTGACGGTA |
| | ACCACCGTAGGCGTGGAGAAGATGATGATCCGTGCCGGCCTGGACGTATCGCG |
| | CTTCGGTCCGCACCTGAAGATCGGCATCGAGCGCGCGGTGGCCTTGCGCATCGA |
| | ACTCAATGCCAAGACCCAGATCGCGCTTTACGGGGGAGTGCTGGTGGAACAGC |
| | GACTGGCGGTTTCA |
| SEQ ID NO. 12 | ATGACCATGATTACGGATTCACTGGCCGTCGTTTTACAACGTCGTGACTGGGAA |
| LacZ | AACCCTGGCGTTACCCAACTTAATCGCCTTGCAGCACATCCCCCTTTCGCCAGCT |
| | GGCGTAATAGCGAAGAGGCCCGCACCGATCGCCCTTCCCAACAGTTGCGCAGCC |
| | TGAATGGCGAATGGCGCTTTGCCTGGTTTCCGGCACCAGAAGCGGTGCCGGAAA |
| | GCTGGCTGGAGTGCGATCTTCCTGAGGCCGATACTGTCGTCGTCCCCTCAAACT |
| | GGCAGATGCACGGTTACGATGCGCCCATCTACACCAACGTGACCTATCCCATTA |
| | CGGTCAATCCGCCGTTTGTTCCCACGGAGAATCCGACGGGTTGTTACTCGCTCA |
| | CATTTAATGTTGATGAAAGCTGGCTACAGGAAGGCCAGACGCGAATTATTTTTG |
| | ATGGCGTTAACTCGGCGTTTCATCTGTGGTGCAACGGGCGCTGGGTCGGTTACG |
| | GCCAGGACAGTCGTTTGCCGTCTGAATTTGACCTGAGCGCATTTTTACGCGCCG |
| | GAGAAAACCGCCTCGCGGTGATGGTGCTGCGCTGGAGTGACGGCAGTTATCTGG |
| | AAGATCAGGATATGTGGCGGATGAGCGGCATTTTCCGTGACGTCTCGTTGCTGC |
| | ATAAACCGACTACACAAATCAGCGATTTCCATGTTGCCACTCGCTTTAATGATG |
| | ATTTCAGCCGCGCTGTACTGGAGGCTGAAGTTCAGATGTGCGGCGAGTTGCGTG |
| | ACTACCTACGGGTAACAGTTTCTTTATGGCAGGGTGAAACGCAGGTCGCCAGCG |
| | GCACCGCGCCTTTCGGCGGTGAAATTATCGATGAGCGTGGTGGTTATGCCGATC |
| | GCGTCACACTACGTCTGAACGTCGAAAACCCGAAACTGTGGAGCGCCGAAATC |
| | CCGAATCTCTATCGTGCGGTGGTTGAACTGCACACCGCCGACGGCACGCTGATT |
| | GAAGCAGAAGCCTGCGATGTCGGTTTCCGCGAGGTGCGGATTGAAAATGGTCTG |
| | CTGCTGCTGAACGGCAAGCCGTTGCTGATTCGAGGCGTTAACCGTCACGAGCAT |
| | CATCCTCTGCATGGTCAGGTCATGGATGAGCAGACGATGGTGCAGGATATCCTG |
| | CTGATGAAGCAGAACAACTTTAACGCCGTGCGCTGTTCGCATTATCCGAACCAT |
| | CCGCTGTGGTACACGCTGTGCGACCGCTACGGCCTGTATGTGGTGGATGAAGCC |
| | AATATTGAAACCCACGGCATGGTGCCAATGAATCGTCTGACCGATGATCCGCGC |
| | TGGCTACCGGCGATGAGCGAACGCGTAACGCGAATGGTGCAGCGCGATCGTAA |
| | TCACCCGAGTGTGATCATCTGGTCGCTGGGGAATGAATCAGGCCACGGCGCTAA |
| | TCACGACGCGCTGTATCGCTGGATCAAATCTGTCGATCCTTCCCGCCCGGTGCA |
| | GTATGAAGGCGGCGGAGCCGACACCACGGCCACCGATATTATTTGCCCGATGTA |
| | CGCGCGCGTGGATGAAGACCAGCCCTTCCCGGCTGTGCCGAAATGGTCCATCAA |
| | AAAATGGCTTTCGCTACCTGGAGAGACGCGCCCGCTGATCCTTTGCGAATACGC |
| | CCACGCGATGGGTAACAGTCTTGGCGGTTTCGCTAAATACTGGCAGGCGTTTCG |
| | TCAGTATCCCCGTTTACAGGGCGGCTTCGTCTGGGACTGGGTGGATCAGTCGCT |
| | GATTAAATATGATGAAAACGGCAACCCGTGGTCGGCTTACGGCGGTGATTTTGG |
| | CGATACGCCGAACGATCGCCAGTTCTGTATGAACGGTCTGGTCTTTGCCGACCG |
| | CACGCCGCATCCAGCGCTGACGGAAGCAAAACACCAGCAGCAGTTTTTCCAGTT |
| | CCGTTTATCCGGGCAAACCATCGAAGTGACCAGCGAATACCTGTTCCGTCATAG |
| | CGATAACGAGCTCCTGCACTGGATGGTGGCGCTGGATGGTAAGCCGCTGGCAA |
| | GCGGTGAAGTGCCTCTGGATGTCGCTCCACAAGGTAAACAGTTGATTGAACTGC |
| | CTGAACTACCGCAGCCGGAGAGCGCCGGGCAACTCTGGCTCACAGTACGCGTA |
| | GTGCAACCGAACGCGACCGCATGGTCAGAAGCCGGGCACATCAGCGCCTGGCA |
| | GCAGTGGCGTCTGGCGGAAAACCTCAGTGTGACGCTCCCCGCCGCGTCCCACGC |
| | CATCCCGCATCTGACCACCAGCGAAATGGATTTTTGCATCGAGCTGGGTAATAA |
| | GCGTTGGCAATTTAACCGCCAGTCAGGCTTTCTTTCACAGATGTGGATTGGCGA |
| | TAAAAAACAACTGCTGACGCCGCTGCGCGATCAGTTCACCCGTGCACCGCTGGA |
| | TAACGACATTGGCGTAAGTGAAGCGACCCGCATTGACCCTAACGCCTGGGTCGA |
| | ACGCTGGAAGGCGGCGGGCCATTACCAGGCCGAAGCAGCGTTGTTGCAGTGCA |
| | CGGCAGATACACTTGCTGATGCGGTGCTGATTACGACCGCTCACGCGTGGCAGC |
| | ATCAGGGGAAAACCTTATTTATCAGCCGGAAAACCTACCGGATTGATGGTAGTG |
| | GTCAAATGGCGATTACCGTTGATGTTGAAGTGGCGAGCGATACACCGCATCCGG |
| | CGCGGATTGGCCTGAACTGCCAGCTGGCGCAGGTAGCAGAGCGGGTAAACTGG |
| | CTCGGATTAGGGCCGCAAGAAAACTATCCCGACCGCCTTACTGCCGCCTGTTTT |
| | GACCGCTGGGATCTGCCATTGTCAGACATGTATACCCCGTACGTCTTCCCGAGC |
| | GAAAACGGTCTGCGCTGCGGGACGCGCGAATTGAATTATGGCCCACACCAGTG |
| | GCGCGGCGACTTCCAGTTCAACATCAGCCGCTACAGTCAACAGCAACTGATGGA |
| | AACCAGCCATCGCCATCTGCTGCACGCGGAAGAAGGCACATGGCTGAATATCG |
| | ACGGTTTCCATATGGGGATTGGTGGCGACGACTCCTGGAGCCCGTCAGTATCGG |
| | CGGAATTCCAGCTGAGCGCCGGTCGCTACCATTACCAGTTGGTCTGGTGTCAAA |
| | AATAA |
| SEQ ID NO. 13 | AAATCTATCTCATTTGCTAGTTATAAAATTATGAAATTTGCATAAATTCTTCA |
| Las promoter | |
| SEQ ID NO. 14 | ATGGCCTTGGTTGACGGTTTTCTTGAGCTGGAACGCTCAAGTGGAAAATTGGAG |
| LasR | TGGAGCGCCATCCTGCAGAAGATGGCGAGCGACCTTGGATTCTCGAAGATCCTG |
| | TTCGGCCTGTTGCCTAAGGACAGCCAGGACTACGAGAACGCCTTCATCGTCGGC |
| | AACTACCCGGCCGCCTGGCGCGAGCATTACGACCGGGCTGGCTACGCGCGGGTC |
| | GACCCGACGGTCAGTCACTGTACCCAGAGCGTACTGCCGATTTTCTGGGAACCG |
| | TCCATCTACCAGACGCGAAAGCAGCACGAGTTCTTCGAGGAAGCCTCGGCCGCC |
| | GGCCTGGTGTATGGGCTGACCATGCCGCTGCATGGTGCTCGCGGCGAACTCGGC |
| | GCGCTGAGCCTCAGCGTGGAAGCGGAAAACCGGGCCGAGGCCAACCGTTTCAT |
| | GGAGTCGGTCCTGCCGACCCTGTGGATGCTCAAGGACTACGCACTGCAGAGCGG |
| | TGCCGGACTGGCCTTCGAACATCCGGTCAGCAAACCGGTGGTTCTGACCAGCCG |
| | GGAGAAGGAAGTGTTGCAGTGGTGCGCCATCGGCAAGACCAGTTGGGAGATAT |
| | CGGTTATCTGCAACTGCTCGGAAGCCAATGTGAACTTCCATATGGGAAATATTC |
| | GGCGGAAGTTCGGTGTGACCTCCCGCCGCGTAGCGGCCATTATGGCCGTTAATT |
| | TGGGTCTTATTACTCTCTGATAATAA |
| SEQ ID NO. 15 | CTCGAGAAATCATAAAAAATTTATTTGCTTTGTTAGCGGAGAAGAATTATAATA |
| mT5 promoter | GATTCAATTGTGAGCGGATAACAA |
| SEQ ID NO. 16 | ATGGATAGCACTGAGAACGTCATCAAGCCCTTCATGCGCTTCAAGGTGCACATG |
| DsRedExpress2 | GAGGGCTCCGTGAACGGCCACGAGTTCGAGATCGAGGGCGAGGGCGAGGGCAA |
| | GCCTTACGAGGGCACCCAGACCGCCAAGCTGCAGGTGACCAAGGGCGGCCCCC |
| | TGCCCTTCGCCTGGGACATCCTGTCCCCCCAGTTCCAGTACGGCTCCAAGGTGTA |
| | CGTGAAGCACCCCGCCGACATCCCCGACTACAAGAAGCTGTCCTTCCCCGAGGG |
| | CTTCAAGTGGGAGCGCGTGATGAACTTCGAGGACGGCGGCGTGGTGACCGTGA |
| | CCCAGGACTCCTCCCTGCAGGACGGCACCTTCATCTACCACGTGAAGTTCATCG |
| | GCGTGAACTTCCCCTCCGACGGCCCCGTAATGCAGAAGAAGACTCTGGGCTGGG |
| | AGCCCTCCACCGAGCGCCTGTACCCCCGCGACGGCGTGCTGAAGGGCGAGATCC |
| | ACAAGGCGCTGAAGCTGAAGGGCGGCGGCCACTACCTGGTGGAGTTCAAGTCA |
| | ATCTACATGGCCAAGAAGCCCGTGAAGCTGCCCGGCTACTACTACGTGGACTCC |
| | AAGCTGGACATCACCTCCCACAACGAGGACTACACCGTGGTGGAGCAGTACGA |
| | GCGCGCCGAGGCCCGCCACCACCTGTTCCAGTGA |
| SEQ ID NO. 17 | GCAGCGAACGACGAAAATTACGCCCTTGCAGCGTGATAATAA |
| LAA ssRA tag | |
| SEQ ID NO. 18 | ATGAAAAAAACTGCTATCGCTATCGCTGTTGCTCTGGCTGGTTTCGCTACTGTTG |
| GMCSF | CTCAGGCGGCGGCCATGGCTGCACCCGCCCGCTCGCCCAGCCCCAGCACACAGC |
| | CCTGGGAGCATGTGAATGCCATCCAGGAGGCCCGGCGTCTCCTGAACCTGAGTA |
| | GAGACACTGCTGCTGAGATGAATGAAACAGTAGAAGTCATCTCAGAAATGTTTG |
| | ACCTCCAGGAGCCGACCTGCCTACAGACCCGCCTGGAGCTGTACAAGCAGGGC |
| | CTGCGGGGCAGCCTCACCAAGCTCAAGGGCCCCTTGACCATGATGGCCAGCCAC |
| | TACAAACAGCACTGCCCTCCAACCCCGGAAACTTCCTGTGCAACCCAGATTATC |
| | ACCTTTGAAAGTTTCAAAGAGAACCTGAAGGACTTTCTGCTTGTCATCCCCTTTG |
| | ACTGCTGGGAGCCAGTCCAGGAGGCGGCCGCTGGATCCGAACAAAAGCTGATC |
| | TCAGAAGAAGACCTAAAGGCCTCGGGGGCCGATCACCATCATCACCATCATTGA |
| SEQ ID NO. 19 | ATGAACATGAAAAAACTGGCTACCCTGGTTTCCGCTGTTGCGCTAAGCGCCACC |
| TolAIII | GTTAGTGCGAATGCGATGGCAAAAGACACCATCGCGGCCGCAGAGGCAGATGA |
| | TATTTTCGGTGAGCTAAGCTCTGGTAAGAATGCACCGAAAACGGGGGGAGGGG |
| | CGAAAGGGAACAATGCTTCGCCTGCCGGGAGTGGTAATACTAAAAACAATGGC |
| | GCATCAGGGGCCGATATCAATAACTATGCCGGGCAGATTAAATCTGCTATCGAA |
| | AGTAAGTTCTATGACGCATCGTCCTATGCAGGCAAAACCTGTACGCTGCGCATA |
| | AAACTGGCACCCGATGGTATGTTACTGGATATCAAACCTGAAGGTGGCGATCCC |
| | GCACTTTGTCAGGCTGCGTTGGCAGCAGCTAAACTTGCGAAGATCCCGAAACCA |
| | CCAAGCCAGGCAGTATATGAAGTGTTCAAAAACGCGCCATTGGACTTCAAACCG |
| | TAA |
| SEQ ID NO. 20 | ATGACCCAGTCACGTAGCGATAGCGGAGTGTATACTGGCTTAACTATGCGGCAT |
| pBR322 | CAGAGCAGATTGTACTGAGAGTGCACCATATGCGGTGTGAAATACCGCACAGA |
| backbone | TGCGTAAGGAGAAAATACCGCATCAGGCGCTCTTCCGCTTCCTCGCTCACTGAC |
| | TCGCTGCGCTCGGTCGTTCGGCTGCGGCGAGCGGTATCAGCTCACTCAAAGGCG |
| | GTAATACGGTTATCCACAGAATCAGGGGATAACGCAGGAAAGAACATGTGAGC |
| | AAAAGGCCAGCAAAAGGCCAGGAACCGTAAAAAGGCCGCGTTGCTGGCGTTTT |
| | TCCATAGGCTCCGCCCCCCTGACGAGCATCACAAAAATCGACGCTCAAGTCAGA |
| | GGTGGCGAAACCCGACAGGACTATAAAGATACCAGGCGTTTCCCCCTGGAAGC |
| | TCCCTCGTGCGCTCTCCTGTTCCGACCCTGCCGCTTACCGGATACCTGTCCGCCT |
| | TTCTCCCTTCGGGAAGCGTGGCGCTTTCTCATAGCTCACGCTGTAGGTATCTCAG |
| | TTCGGTGTAGGTCGTTCGCTCCAAGCTGGGCTGTGTGCACGAACCCCCCGTTCA |
| | GCCCGACCGCTGCGCCTTATCCGGTAACTATCGTCTTGAGTCCAACCCGGTAAG |
| | ACACGACTTATCGCCACTGGCAGCAGCCACTGGTAACAGGATTAGCAGAGCGA |
| | GGTATGTAGGCGGTGCTACAGAGTTCTTGAAGTGGTGGCCTAACTACGGCTACA |
| | CTAGAAGGACAGTATTTGGTATCTGCGCTCTGCTGAAGCCAGTTACCTTCGGAA |
| | AAAGAGTTGGTAGCTCTTGATCCGGCAAACAAACCACCGCTGGTAGCGGTGGTT |
| | TTTTTGTTTGCAAGCAGCAGATTACGCGCAGAAAAAAAGGATCTCAAGAAGATC |
| | CTTTGATCTTTTCTACGGGGTCTGACGCTCAGTGGAACGAAAACTCACGTTAAG |
| | GGATTTTGGTCATGAGATTATCAAAAAGGATCTTCACCTAGATCCTTTTAAATTA |
| | AAAATGAAGTTTTAAATCAATCTAAAGTATATATGAGTAAACTTGGTCTGACAG |
| | TTACCAATGCTTAATCAGTGAGGCACCTATCTCAGCGATCTGTCTATTTCGTTCA |
| | TCCATAGTTGCCTGACTCCCCGTCGTGTAGATAACTACGATACGGGAGGGCTTA |
| | CCATCTGGCCCCAGTGCTGCAATGATACCGCGAGACCCACGCTCACCGGCTCCA |
| | GATTTATCAGCAATAAACCAGCCAGCCGGAAGGGCCGAGCGCAGAAGTGGTCC |
| | TGCAACTTTATCCGCCTCCATCCAGTCTATTAATTGTTGCCGGGAAGCTAGAGTA |
| | AGTAGTTCGCCAGTTAATAGTTTGCGCAACGTTGTTGCCATTGCTGCAGGCATC |
| | GTGGTGTCACGCTCGTCGTTTGGTATGGCTTCATTCAGCTCCGGTTCCCAACGAT |
| | CAAGGCGAGTTACATGATCCCCCATGTTGTGCAAAAAAGCGGTTAGCTCCTTCG |
| | GTCCTCCGATCGTTGTCAGAAGTAAGTTGGCCGCAGTGTTATCACTCATGGTTAT |
| | GGCAGCACTGCATAATTCTCTTACTGTCATGCCATCCGTAAGATGCTTTTCTGTG |
| | ACTGGTGAGTACTCAACCAAGTCATTCTGAGAATAGTGTATGCGGCGACCGAGT |
| | TGCTCTTGCCCGGCGTCAACACGGGATAATACCGCGCCACATAGCAGAACTTTA |
| | AAAGTGCTCATCATTGGAAAACGTTCTTCGGGGCGAAAACTCTCAAGGATCTTA |
| | CCGCTGTTGAGATCCAGTTCGATGTAACCCACTCGTGCACCCAACTGATCTTCA |
| | GCATCTTTTACTTTCACCAGCGTTTCTGGGTGAGCAAAAACAGGAAGGCAAAAT |
| | GCCGCAAAAAAGGGAATAAGGGCGACACGGAAATGTTGAATACTCATACTCTT |
| | CCTTTTTCAATATTATTGAAGCATTTATCAGGGTTATTGTCTCATGAGCGGATAC |
| | ATATTTGAATGTATTTAGAAAAATAAACAAATAGGGGTTCCGCGCACATTTCCC |
| | CGAAAAGTGCCACCTGACGTCTAAGAAACCATTATTATCATGACATTAACCTAT |
| | AAAAATAGGCGTATCACGAGGCCCTTTCGTCTTCAAGAATTCTCATGTTTGACA |
| | GCTTATCATCGATAAGCTTTGCGGTAGTTTATCACAGTTAAATTGCTAACGCAGT |
| | CAGGCACCGTGTATGAAATCTAACAATGCGCTCATCGTCATCCTCGGCACCGTC |
| | ACCCTGGATGCTGTAGGCATAGGCTTGGTTATGCCGGTACTGCCGGGCCTCTTG |
| | CGGGATATCGTCCATTCCGACAGCATCGCCAGTCACTATGGCGTGCTGCTAGCG |
| | CTATATGCGTTGATGCAATTTCTATGCGCACCCGTTCTCGGAGCACTGTCCGACC |
| | GCTTTGGCCGCCGCCCAGTCCTGCTCGCTTCGCTACTTGGAGCCACTATCGACTA |
| | CGCGATCATGGCGACCACACCCGTCCTGTGGATCCTCTACGCCGGACGCATCGT |
| | GGCCGGCATCACCGGCGCCACAGGTGCGGTTGCTGGCGCCTATATCGCCGACAT |
| | CACCGATGGGGAAGATCGGGCTCGCCACTTCGGGCTCATGAGCGCTTGTTTCGG |
| | CGTGGGTATGGTGGCAGGCCCCGTGGCCGGGGGACTGTTGGGCGCCATCTCCTT |
| | GCATGCACCATTCCTTGCGGCGGCGGTGCTCAACGGCCTCAACCTACTACTGGG |
| | CTGCTTCCTAATGCAGGAGTCGCATAAGGGAGAGCGTCGACCGATGCCCTTGAG |
| | AGCCTTCAACCCAGTCAGCTCCTTCCGGTGGGCGCGGGGCATGACTATCGTCGC |
| | CGCACTTATGACTGTCTTCTTTATCATGCAACTCGTAGGACAGGTGCCGGCAGC |
| | GCTCTGGGTCATTTTCGGCGAGGACCGCTTTCGCTGGAGCGCGACGATGATCGG |
| | CCTGTCGCTTGCGGTATTCGGAATCTTGCACGCCCTCGCTCAAGCCTTCGTCACT |
| | GGTCCCGCCACCAAACGTTTCGGCGAGAAGCAGGCCATTATCGCCGGCATGGCG |
| | GCCGACGCGCTGGGCTACGTCTTGCTGGCGTTCGCGACGCGAGGCTGGATGGCC |
| | TTCCCCATTATGATTCTTCTCGCTTCCGGCGGCATCGGGATGCCCGCGTTGCAGG |
| | CCATGCTGTCCAGGCAGGTAGATGACGACCATCAGGGACAGCTTCAAGGATCG |
| | CTCGCGGCTCTTACCAGCCTAACTTCGATCATTGGACCGCTGATCGTCACGGCG |
| | ATTTATGCCGCCTCGGCGAGCACATGGAACGGGTTGGCATGGATTGTAGGCGCC |
| | GCCCTATACCTTGTCTGCCTCCCCGCGTTGCGTCGCGGTGCATGGAGCCGGGCC |
| | ACCTCGACCTGAATGGAAGCCGGCGGCACCTCGCTAACGGATTCACCACTCCAA |
| | GAATTGGAGCCAATCAATTCTTGCGGAGAACTGTGAATGCGCAAACCAACCCTT |
| | GGCAGAACATATCCATCGCGTCCGCCATCTCCAGCAGCCGCACGCGGCGCATCT |
| | CGGGCAGCGTTGGGTCCTGGCCACGGGTGCGCATGATCGTGCTCCTGTCGTTGA |
| | GGACCCGGCTAGGCTGGCGGGGTTGCCTTACTGGTTAGCAGAATGAATCACCGA |
| | TACGCGAGCGAACGTGAAGCGACTGCTGCTGCAAAACGTCTGCGACCTGAGCA |
| | ACAACATGAATGGTCTTCGGTTTCCGTGTTTCGTAAAGTCTGGAAACGCGGAAG |
| | TCAGCGCCCTGCACCATTATGTTCCGGATCTGCATCGCAGGATGCTGCTGGCTA |
| | CCCTGTGGAACACCTACATCTGTATTAACGAAGCGCTGGCATTGACCCTGAGTG |
| | ATTTTTCTCTGGTCCCGCCGCATCCATACCGCCAGTTGTTTACCCTCACAACGTT |
| | CCAGTAACCGGGCATGTTCATCATCAGTAACCCGTATCGTGAGCATCCTCTCTC |
| | GTTTCATCGGTATCATTACCCCCATGAACAGAAATCCCCCTTACACGGAGGCAT |
| | CAGTGACCAAACAGGAAAAAACCGCCCTTAACATGGCCCGCTTTATCAGAAGC |
| | CAGACATTAACGCTTCTGGAGAAACTCAACGAGCTGGACGCGGATGAACAGGC |
| | AGACATCTGTGAATCGCTTCACGACCACGCTGATGAGCTTTACCGCAGCTGCCT |
| | CGCGCGTTTCGGTGATGACGGTGAAAACCTCTGACACATGCAGCTCCCGGAGAC |
| | GGTCACAGCTTGTCTGTAAGCGGATGCCGGGAGCAGACAAGCCCGTCAGGGCG |
| | CGTCAGCGGGTGTTGGCGGGTGTCGGGGCGCAGCC |
| SEQ ID NO. 21 | ATGACCCAGTCACGTAGCGATAGCGGAGTGTATACTGGCTTAACTATGCGGCAT |
| pACYC184 | CAGAGCAGATTGTACTGAGAGTGCACCATATGCGGTGTGAAATACCGCACAGA |
| backbone | TGCGTAAGGAGAAAATACCGCATCAGGCGCTCTTCCGCTTCCTCGCTCACTGAC |
| | TCGCTGCGCTCGGTCGTTCGGCTGCGGCGAGCGGTATCAGCTCACTCAAAGGCG |
| | GTAATACGGTTATCCACAGAATCAGGGGATAACGCAGGAAAGAACATGTGAGC |
| | AAAAGGCCAGCAAAAGGCCAGGAACCGTAAAAAGGCCGCGTTGCTGGCGTTTT |
| | TCCATAGGCTCCGCCCCCCTGACGAGCATCACAAAAATCGACGCTCAAGTCAGA |
| | GGTGGCGAAACCCGACAGGACTATAAAGATACCAGGCGTTTCCCCCTGGAAGC |
| | TCCCTCGTGCGCTCTCCTGTTCCGACCCTGCCGCTTACCGGATACCTGTCCGCCT |
| | TTCTCCCTTCGGGAAGCGTGGCGCTTTCTCATAGCTCACGCTGTAGGTATCTCAG |
| | TTCGGTGTAGGTCGTTCGCTCCAAGCTGGGCTGTGTGCACGAACCCCCCGTTCA |
| | GCCCGACCGCTGCGCCTTATCCGGTAACTATCGTCTTGAGTCCAACCCGGTAAG |
| | ACACGACTTATCGCCACTGGCAGCAGCCACTGGTAACAGGATTAGCAGAGCGA |
| | GGTATGTAGGCGGTGCTACAGAGTTCTTGAAGTGGTGGCCTAACTACGGCTACA |
| | CTAGAAGGACAGTATTTGGTATCTGCGCTCTGCTGAAGCCAGTTACCTTCGGAA |
| | AAAGAGTTGGTAGCTCTTGATCCGGCAAACAAACCACCGCTGGTAGCGGTGGTT |
| | TTTTTGTTTGCAAGCAGCAGATTACGCGCAGAAAAAAAGGATCTCAAGAAGATC |
| | CTTTGATCTTTTCTACGGGGTCTGACGCTCAGTGGAACGAAAACTCACGTTAAG |
| | GGATTTTGGTCATGAGATTATCAAAAAGGATCTTCACCTAGATCCTTTTAAATTA |
| | AAAATGAAGTTTTAAATCAATCTAAAGTATATATGAGTAAACTTGGTCTGACAG |
| | TTACCAATGCTTAATCAGTGAGGCACCTATCTCAGCGATCTGTCTATTTCGTTCA |
| | TCCATAGTTGCCTGACTCCCCGTCGTGTAGATAACTACGATACGGGAGGGCTTA |
| | CCATCTGGCCCCAGTGCTGCAATGATACCGCGAGACCCACGCTCACCGGCTCCA |
| | GATTTATCAGCAATAAACCAGCCAGCCGGAAGGGCCGAGCGCAGAAGTGGTCC |
| | TGCAACTTTATCCGCCTCCATCCAGTCTATTAATTGTTGCCGGGAAGCTAGAGTA |
| | AGTAGTTCGCCAGTTAATAGTTTGCGCAACGTTGTTGCCATTGCTGCAGGCATC |
| | GTGGTGTCACGCTCGTCGTTTGGTATGGCTTCATTCAGCTCCGGTTCCCAACGAT |
| | CAAGGCGAGTTACATGATCCCCCATGTTGTGCAAAAAAGCGGTTAGCTCCTTCG |
| | GTCCTCCGATCGTTGTCAGAAGTAAGTTGGCCGCAGTGTTATCACTCATGGTTAT |
| | GGCAGCACTGCATAATTCTCTTACTGTCATGCCATCCGTAAGATGCTTTTCTGTG |
| | ACTGGTGAGTACTCAACCAAGTCATTCTGAGAATAGTGTATGCGGCGACCGAGT |
| | TGCTCTTGCCCGGCGTCAACACGGGATAATACCGCGCCACATAGCAGAACTTTA |
| | AAAGTGCTCATCATTGGAAAACGTTCTTCGGGGCGAAAACTCTCAAGGATCTTA |
| | CCGCTGTTGAGATCCAGTTCGATGTAACCCACTCGTGCACCCAACTGATCTTCA |
| | GCATCTTTTACTTTCACCAGCGTTTCTGGGTGAGCAAAAACAGGAAGGCAAAAT |
| | GCCGCAAAAAAGGGAATAAGGGCGACACGGAAATGTTGAATACTCATACTCTT |
| | CCTTTTTCAATATTATTGAAGCATTTATCAGGGTTATTGTCTCATGAGCGGATAC |
| | ATATTTGAATGTATTTAGAAAAATAAACAAATAGGGGTTCCGCGCACATTTCCC |
| | CGAAAAGTGCCACCTGACGTCTAAGAAACCATTATTATCATGACATTAACCTAT |
| | AAAAATAGGCGTATCACGAGGCCCTTTCGTCTTCAAGAATTCTCATGTTTGACA |
| | GCTTATCATCGATAAGCTTTGCGGTAGTTTATCACAGTTAAATTGCTAACGCAGT |
| | CAGGCACCGTGTATGAAATCTAACAATGCGCTCATCGTCATCCTCGGCACCGTC |
| | ACCCTGGATGCTGTAGGCATAGGCTTGGTTATGCCGGTACTGCCGGGCCTCTTG |
| | CGGGATATCGTCCATTCCGACAGCATCGCCAGTCACTATGGCGTGCTGCTAGCG |
| | CTATATGCGTTGATGCAATTTCTATGCGCACCCGTTCTCGGAGCACTGTCCGACC |
| | GCTTTGGCCGCCGCCCAGTCCTGCTCGCTTCGCTACTTGGAGCCACTATCGACTA |
| | CGCGATCATGGCGACCACACCCGTCCTGTGGATCCTCTACGCCGGACGCATCGT |
| | GGCCGGCATCACCGGCGCCACAGGTGCGGTTGCTGGCGCCTATATCGCCGACAT |
| | CACCGATGGGGAAGATCGGGCTCGCCACTTCGGGCTCATGAGCGCTTGTTTCGG |
| | CGTGGGTATGGTGGCAGGCCCCGTGGCCGGGGGACTGTTGGGCGCCATCTCCTT |
| | GCATGCACCATTCCTTGCGGCGGCGGTGCTCAACGGCCTCAACCTACTACTGGG |
| | CTGCTTCCTAATGCAGGAGTCGCATAAGGGAGAGCGTCGACCGATGCCCTTGAG |
| | AGCCTTCAACCCAGTCAGCTCCTTCCGGTGGGCGCGGGGCATGACTATCGTCGC |
| | CGCACTTATGACTGTCTTCTTTATCATGCAACTCGTAGGACAGGTGCCGGCAGC |
| | GCTCTGGGTCATTTTCGGCGAGGACCGCTTTCGCTGGAGCGCGACGATGATCGG |
| | CCTGTCGCTTGCGGTATTCGGAATCTTGCACGCCCTCGCTCAAGCCTTCGTCACT |
| | GGTCCCGCCACCAAACGTTTCGGCGAGAAGCAGGCCATTATCGCCGGCATGGCG |
| | GCCGACGCGCTGGGCTACGTCTTGCTGGCGTTCGCGACGCGAGGCTGGATGGCC |
| | TTCCCCATTATGATTCTTCTCGCTTCCGGCGGCATCGGGATGCCCGCGTTGCAGG |
| | CCATGCTGTCCAGGCAGGTAGATGACGACCATCAGGGACAGCTTCAAGGATCG |
| | CTCGCGGCTCTTACCAGCCTAACTTCGATCATTGGACCGCTGATCGTCACGGCG |
| | ATTTATGCCGCCTCGGCGAGCACATGGAACGGGTTGGCATGGATTGTAGGCGCC |
| | GCCCTATACCTTGTCTGCCTCCCCGCGTTGCGTCGCGGTGCATGGAGCCGGGCC |
| | ACCTCGACCTGAATGGAAGCCGGCGGCACCTCGCTAACGGATTCACCACTCCAA |
| | GAATTGGAGCCAATCAATTCTTGCGGAGAACTGTGAATGCGCAAACCAACCCTT |
| | GGCAGAACATATCCATCGCGTCCGCCATCTCCAGCAGCCGCACGCGGCGCATCT |
| | CGGGCAGCGTTGGGTCCTGGCCACGGGTGCGCATGATCGTGCTCCTGTCGTTGA |
| | GGACCCGGCTAGGCTGGCGGGGTTGCCTTACTGGTTAGCAGAATGAATCACCGA |
| | TACGCGAGCGAACGTGAAGCGACTGCTGCTGCAAAACGTCTGCGACCTGAGCA |
| | ACAACATGAATGGTCTTCGGTTTCCGTGTTTCGTAAAGTCTGGAAACGCGGAAG |
| | TCAGCGCCCTGCACCATTATGTTCCGGATCTGCATCGCAGGATGCTGCTGGCTA |
| | CCCTGTGGAACACCTACATCTGTATTAACGAAGCGCTGGCATTGACCCTGAGTG |
| | ATTTTTCTCTGGTCCCGCCGCATCCATACCGCCAGTTGTTTACCCTCACAACGTT |
| | CCAGTAACCGGGCATGTTCATCATCAGTAACCCGTATCGTGAGCATCCTCTCTC |
| | GTTTCATCGGTATCATTACCCCCATGAACAGAAATCCCCCTTACACGGAGGCAT |
| | CAGTGACCAAACAGGAAAAAACCGCCCTTAACATGGCCCGCTTTATCAGAAGC |
| | CAGACATTAACGCTTCTGGAGAAACTCAACGAGCTGGACGCGGATGAACAGGC |
| | AGACATCTGTGAATCGCTTCACGACCACGCTGATGAGCTTTACCGCAGCTGCCT |
| | CGCGCGTTTCGGTGATGACGGTGAAAACCTCTGACACATGCAGCTCCCGGAGAC |
| | GGTCACAGCTTGTCTGTAAGCGGATGCCGGGAGCAGACAAGCCCGTCAGGGCG |
| | CGTCAGCGGGTGTTGGCGGGTGTCGGGGCGCAGCC |
| SEQ ID NO. 22 | TGGCGAATGGGACGCGCCCTGTAGCGGCGCATTAAGCGCGGCGGGTGTGGTGG |
| pET21 backbone | TTACGCGCAGCGTGACCGCTACACTTGCCAGCGCCCTAGCGCCCGCTCCTTTCG |
| | CTTTCTTCCCTTCCTTTCTCGCCACGTTCGCCGGCTTTCCCCGTCAAGCTCTAAAT |
| | CGGGGGCTCCCTTTAGGGTTCCGATTTAGTGCTTTACGGCACCTCGACCCCAAA |
| | AAACTTGATTAGGGTGATGGTTCACGTAGTGGGCCATCGCCCTGATAGACGGTT |
| | TTTCGCCCTTTGACGTTGGAGTCCACGTTCTTTAATAGTGGACTCTTGTTCCAAA |
| | CTGGAACAACACTCAACCCTATCTCGGTCTATTCTTTTGATTTATAAGGGATTTT |
| | GCCGATTTCGGCCTATTGGTTAAAAAATGAGCTGATTTAACAAAAATTTAACGC |
| | GAATTTTAACAAAATATTAACGTTTACAATTTCAGGTGGCACTTTTCGGGGAAA |
| | TGTGCGCGGAACCCCTATTTGTTTATTTTTCTAAATACATTCAAATATGTATCCG |
| | CTCATGAGACAATAACCCTGATAAATGCTTCAATAATATTGAAAAAGGAAGAGT |
| | ATGAGTATTCAACATTTCCGTGTCGCCCTTATTCCCTTTTTTGCGGCATTTTGCCT |
| | TCCTGTTTTTGCTCACCCAGAAACGCTGGTGAAAGTAAAAGATGCTGAAGATCA |
| | GTTGGGTGCACGAGTGGGTTACATCGAACTGGATCTCAACAGCGGTAAGATCCT |
| | TGAGAGTTTTCGCCCCGAAGAACGTTTTCCAATGATGAGCACTTTTAAAGTTCTG |
| | CTATGTGGCGCGGTATTATCCCGTATTGACGCCGGGCAAGAGCAACTCGGTCGC |
| | CGCATACACTATTCTCAGAATGACTTGGTTGAGTACTCACCAGTCACAGAAAAG |
| | CATCTTACGGATGGCATGACAGTAAGAGAATTATGCAGTGCTGCCATAACCATG |
| | AGTGATAACACTGCGGCCAACTTACTTCTGACAACGATCGGAGGACCGAAGGA |
| | GCTAACCGCTTTTTTGCACAACATGGGGGATCATGTAACTCGCCTTGATCGTTGG |
| | GAACCGGAGCTGAATGAAGCCATACCAAACGACGAGCGTGACACCACGATGCC |
| | TGCAGCAATGGCAACAACGTTGCGCAAACTATTAACTGGCGAACTACTTACTCT |
| | AGCTTCCCGGCAACAATTAATAGACTGGATGGAGGCGGATAAAGTTGCAGGAC |
| | CACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCC |
| | CTCCCGTATCGTAGTTATCTACACGACGGGGAGTCAGGCAACTATGGATGAACG |
| | AAATAGACAGATCGCTGAGATAGGTGCCTCACTGATTAAGCATTGGTAACTGTC |
| | AGACCAAGTTTACTCATATATACTTTAGATTGATTTAAAACTTCATTTTTAATTT |
| | AAAAGGATCTAGGTGAAGATCCTTTTTGATAATCTCATGACCAAAATCCCTTAA |
| | CGTGAGTTTTCGTTCCACTGAGCGTCAGACCCCGTAGAAAAGATCAAAGGATCT |
| | TCTTGAGATCCTTTTTTTCTGCGCGTAATCTGCTGCTTGCAAACAAAAAAACCAC |
| | CGCTACCAGCGGTGGTTTGTTTGCCGGATCAAGAGCTACCAACTCTTTTTCCGAA |
| | GGTAACTGGCTTCAGCAGAGCGCAGATACCAAATACTGTCCTTCTAGTGTAGCC |
| | GTAGTTAGGCCACCACTTCAAGAACTCTGTAGCACCGCCTACATACCTCGCTCT |
| | GCTAATCCTGTTACCAGTGGCTGCTGCCAGTGGCGATAAGTCGTGTCTTACCGG |
| | GTTGGACTCAAGACGATAGTTACCGGATAAGGCGCAGCGGTCGGGCTGAACGG |
| | GGGGTTCGTGCACACAGCCCAGCTTGGAGCGAACGACCTACACCGAACTGAGA |
| | TACCTACAGCGTGAGCTATGAGAAAGCGCCACGCTTCCCGAAGGGAGAAAGGC |
| | GGACAGGTATCCGGTAAGCGGCAGGGTCGGAACAGGAGAGCGCACGAGGGAG |
| | CTTCCAGGGGGAAACGCCTGGTATCTTTATAGTCCTGTCGGGTTTCGCCACCTCT |
| | GACTTGAGCGTCGATTTTTGTGATGCTCGTCAGGGGGGCGGAGCCTATGGAAAA |
| | ACGCCAGCAACGCGGCCTTTTTACGGTTCCTGGCCTTTTGCTGGCCTTTTGCTCA |
| | CATGTTCTTTCCTGCGTTATCCCCTGATTCTGTGGATAACCGTATTACCGCCTTTG |
| | AGTGAGCTGATACCGCTCGCCGCAGCCGAACGACCGAGCGCAGCGAGTCAGTG |
| | AGCGAGGAAGCGGAAGAGCGCCTGATGCGGTATTTTCTCCTTACGCATCTGTGC |
| | GGTATTTCACACCGCATATATGGTGCACTCTCAGTACAATCTGCTCTGATGCCGC |
| | ATAGTTAAGCCAGTATACACTCCGCTATCGCTACGTGACTGGGTCATGGCTGCG |
| | CCCCGACACCCGCCAACACCCGCTGACGCGCCCTGACGGGCTTGTCTGCTCCCG |
| | GCATCCGCTTACAGACAAGCTGTGACCGTCTCCGGGAGCTGCATGTGTCAGAGG |
| | TTTTCACCGTCATCACCGAAACGCGCGAGGCAGCTGCGGTAAAGCTCATCAGCG |
| | TGGTCGTGAAGCGATTCACAGATGTCTGCCTGTTCATCCGCGTCCAGCTCGTTGA |
| | GTTTCTCCAGAAGCGTTAATGTCTGGCTTCTGATAAAGCGGGCCATGTTAAGGG |
| | CGGTTTTTTCCTGTTTGGTCACTGATGCCTCCGTGTAAGGGGGATTTCTGTTCAT |
| | GGGGGTAATGATACCGATGAAACGAGAGAGGATGCTCACGATACGGGTTACTG |
| | ATGATGAACATGCCCGGTTACTGGAACGTTGTGAGGGTAAACAACTGGCGGTAT |
| | GGATGCGGCGGGACCAGAGAAAAATCACTCAGGGTCAATGCCAGCGCTTCGTT |
| | AATACAGATGTAGGTGTTCCACAGGGTAGCCAGCAGCATCCTGCGATGCAGATC |
| | CGGAACATAATGGTGCAGGGCGCTGACTTCCGCGTTTCCAGACTTTACGAAACA |
| | CGGAAACCGAAGACCATTCATGTTGTTGCTCAGGTCGCAGACGTTTTGCAGCAG |
| | CAGTCGCTTCACGTTCGCTCGCGTATCGGTGATTCATTCTGCTAACCAGTAAGGC |
| | AACCCCGCCAGCCTAGCCGGGTCCTCAACGACAGGAGCACGATCATGCGCACC |
| | CGTGGGGCCGCCATGCCGGCGATAATGGCCTGCTTCTCGCCGAAACGTTTGGTG |
| | GCGGGACCAGTGACGAAGGCTTGAGCGAGGGCGTGCAAGATTCCGAATACCGC |
| | AAGCGACAGGCCGATCATCGTCGCGCTCCAGCGAAAGCGGTCCTCGCCGAAAA |
| | TGACCCAGAGCGCTGCCGGCACCTGTCCTACGAGTTGCATGATAAAGAAGACA |
| | GTCATAAGTGCGGCGACGATAGTCATGCCCCGCGCCCACCGGAAGGAGCTGAC |
| | TGGGTTGAAGGCTCTCAAGGGCATCGGTCGAGATCCCGGTGCCTAATGAGTGAG |
| | CTAACTTACATTAATTGCGTTGCGCTCACTGCCCGCTTTCCAGTCGGGAAACCTG |
| | TCGTGCCAGCTGCATTAATGAATCGGCCAACGCGCGGGGAGAGGCGGTTTGCGT |
| | ATTGGGCGCCAGGGTGGTTTTTCTTTTCACCAGTGAGACGGGCAACAGCTGATT |
| | GCCCTTCACCGCCTGGCCCTGAGAGAGTTGCAGCAAGCGGTCCACGCTGGTTTG |
| | CCCCAGCAGGCGAAAATCCTGTTTGATGGTGGCATGCGAATTCTTATTTGTAGA |
| | GCTCGGTTAATTTCTCCTCTTGAATTCACTGGCCGTCGTTTTACAGGATCTTGTA |
| | CAGGGCCCTTAGAATTCCACCACCACCACCACCACTGAGATCCGGCTGCTAACA |
| | AAGCCCGAAAGGAAGCTGAGTTGGCTGCTGCCACCGCTGAGCAATAACTAGCA |
| | TAACCCCTTGGGGCCTCTAAACGGGTCTTGAGGGGTTTTTTGCTGAAAGGAGGA |
| | ACTATATCCGGAT |

TABLE 4
Primers used in this study.
Below, capital nucleotides indicate homology with
template.

| Oligomer Name | Sequence |
|---|---|
| SEQ ID NO. 23 | AAGCTTATCGATGATAAGCTG |
| pBR322-1 | |
| SEQ ID NO. 24 | TAATGCGGTAGTTTATCACAG |
| pBR322-2 | |
| SEQ ID NO. 25 | cttatcatcgataagcttTTAAACCGCCTGT |
| OxyRS-1 | TTTAAAAC |
| SEQ ID NO. 26 | cctttgctCATATGTATATCTCCTTCTTAAA |
| OxyRS-2 | GTTAAAC |
| SEQ ID NO. 27 | tatacatatgAGCAAAGGAGAAGAACTTTTC |
| sfGFP1aa-1 | |
| SEQ ID NO. 28 | tgataaactaccgcattaTTATTATCACGCT |
| sfGFP1aa-2 | GCAAGG |
| SEQ ID NO. 29 | TTCATTATCCATCCTCCATCGCCACGATAG |
| pOxyGFP-1 | |
| SEQ ID NO. 30 | TACCTGGTGGCATTGGCTG |
| pOxyGFP-2 | |
| SEQ ID NO. 31 | ccaatgccaccaggtaCTCAAGATCACGAAT |
| proD-1 | ATTCATGG |
| SEQ ID NO. 32 | ggaggatggataatgaaCACAGCTAACACCA |
| proD-2 | CGTC |
| SEQ ID NO. 33 | TAATGCGGTAGTTTATCACAGTTAAATTGCT |
| PoxyAssem-F | AACGCAG |
| SEQ ID NO. 34 | GGTACCTTTCTCCTCTTTAATGAATTCGTGT |
| PoxyAssem-R | GAG |
| SEQ ID NO. 35 | GCAGCGAACGACGAAAATTACGC |
| ssrA-F | |
| SEQ ID NO. 36 | gaggagaaaggtaccATGACCATGATTACGG |
| Poxy-LacZ-F | ATTCACTGGC |
| SEQ ID NO. 37 | gataaactaccgcaTTATTTTTGACACCAGA |
| Poxy-LacZ-R | CCAACTGGTAATGGTAG |
| SEQ ID NO. 38 | ttcgtcgttcgctgcTTTTTGACACCAGACC |
| LacZ-ssrA-R | AACTGGTAATGGTAG |
| SEQ ID NO. 39 | gaggagaaaggtaccATGATCGTACAAATTG |
| Poxy-LasI-F | GTCGGCGC |
| SEQ ID NO. 40 | gataaactaccgcaTTATGAAACCGCCAGTC |
| Poxy-LasI-R | GCTG |
| SEQ ID NO. 41 | ttcgtcgttcgctgcTGAAACCGCCAGTCGC |
| LasI-ssrA-R | TG |
| SEQ ID NO. 42 | aatattgaaaaaggaagagtATGAATAAGGC |
| SigPep_pBla- | CTACAGTATCATTTGGAGCCA |
| ovhg-F | |
| SEQ ID NO. 43 | tttgacagcttatcatcgatGCTAGCTCAGA |
| AIDA_pBla-ovhg- | AGCTGTATTTTATCCCCAGTGCTC |
| R | |
| SEQ ID NO. 44 | ATCGATGATAAGCTGTCAAACATGAGAATTA |
| pBla_3end -F | CAAC |
| SEQ ID NO. 45 | ACTCTTCCTTTTTCAATATTATTGAAGCATT |
| pBla_5end -R | TATCAGGGTTATTG |
| SEQ ID NO. 46 | TGCATTTGCAGTCGACATGCACG |
| GBP3assem-F | |
| SEQ ID NO. 47 | CATTGTTATTCACGGTACCATGATGGTG |
| GBP3assem-R | |
| SEQ ID NO. 48 | TGCAAATGCATTTCCGATTGTGGAAACAACC |
| SurfDispIns-R | G |
| SEQ ID NO. 49 | GGTACCGTGAATAACAATGGAAGCATTGTCA |
| SurfDispIns-F | TTAATAAC |
| SEQ ID NO. 50 | GGTAGCGGCAGCCACCATC |
| GSHis6-F | |
| SEQ ID NO. 51 | GGTACCGTGAATAACAATGGAAGCATTGTCA |
| KpnI-Linker-F | TTAATAAC |
| SEQ ID NO. 52 | GAATTCGCATGCCACCATCAAAC |
| pAHLassem-F | |
| SEQ ID NO. 53 | CTTCACTTCCTCCAAATAG |
| pAHLassem-R | |
| SEQ ID NO. 54 | tgcatactagtATGAGCAAAGGAGAAGAACT |
| sfGFP_SpeI-F | TTTCACTGG |
| SEQ ID NO. 55 | ttcttagagctcggatccatccaTGCCATGT |
| sfGFP_BamHISacI-R | GTAATCCCAG |
| SEQ ID NO. 56 | atttggaggaagtgaagATGAAAAAAACTGC |
| pAHL-TolAIII-F | TATCGC |
| SEQ ID NO. 57 | ggtggcatgcgaattcTTACGGTTTGAAGTC |
| pAHL-TolAIII-R | CAATG |
| SEQ ID NO. 58 | agcatactagtATGACCATGATTACGGATTC |
| LacZ SpeI-F | ACTG |
| SEQ ID NO. 59 | tcttaggatccTTATTTTTGACACCAGACCA |
| LacZ_BamHI-R | ACTG |

TABLE 5
Rate constants for modeling protein kinetics.

| Rate Constant | Value | |
|---|---|---|
| k₁ | 2.2 | OD⁻¹ · min⁻¹ |
| k₂ | 0.015 | μM⁻¹ · min⁻¹ |
| k₃ | 14 | min⁻¹ |
| k₄ | 0.018 [OxyR(o)]⁻¹min⁻¹ | |
| k₅_{_{—}}_{gfp} | 0 | |
| k₅_{_{—}}_{βgal} | 0.00046 | min⁻¹ |
| k₅_{_{—}}_{lasI} | 0.0007 | min⁻¹ |
| k₅_{_{—}}_{AHL} | 2.01 | min⁻¹ |
| k₆_{_{—}}_{gfp} | 0.007 | min⁻¹ |
| k₆_{_{—}}_{βgal} | k_{double} | |
| k₆_{_{—}}_{lasI} | 0.007 | min⁻¹ |
| k_{S}_{_{—}}_{gfp} | 0.4 | |
| k_{S}_{_{—}}_{βgal} | 0.3 | |
| k_{S}_{_{—}}_{lasI} | 0.3 | |
| k_{double} | 0.0115 | min⁻¹ |

| | |
|---|---|
| k₇_{_{—}}_{gfp} | 49,082 |
| k₇_{_{—}}_{lacZ} | 73,170.73 |
| k₇_{_{—}}_{lasI} | 22,000 |
| k₇_{_{—}}_{AHL} | 67.63 |
| τ | 2.47 |
| σ | 0.35 |
| k₈_{_{—}}_{GFP} | 0.1 |

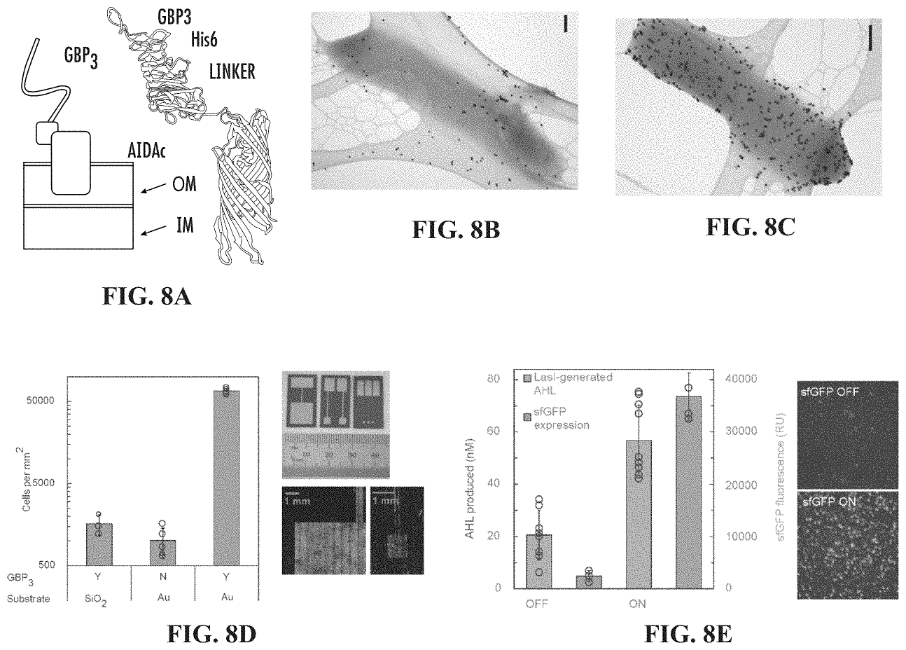

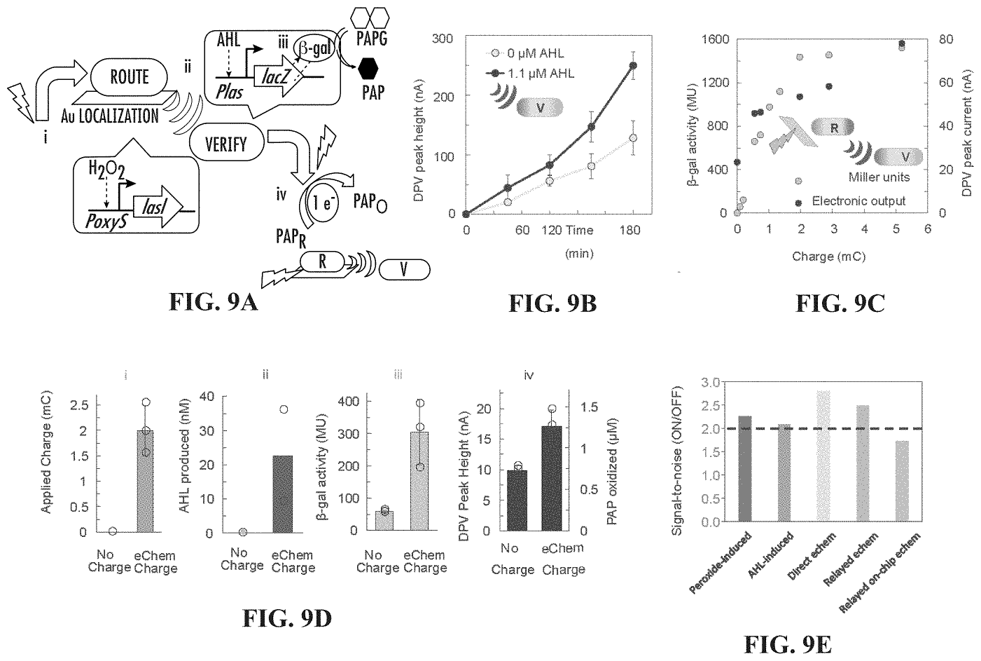

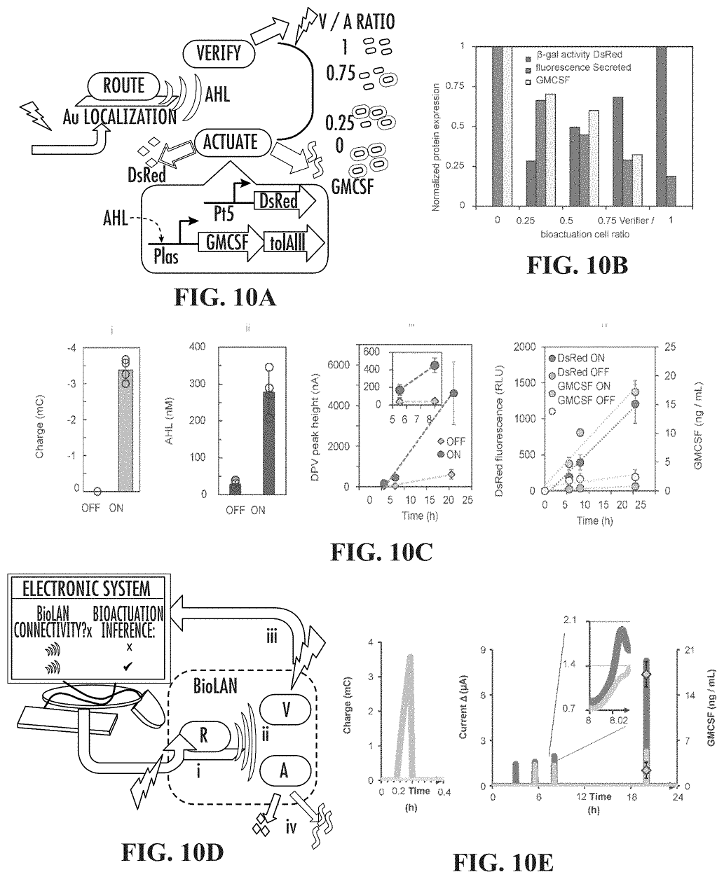

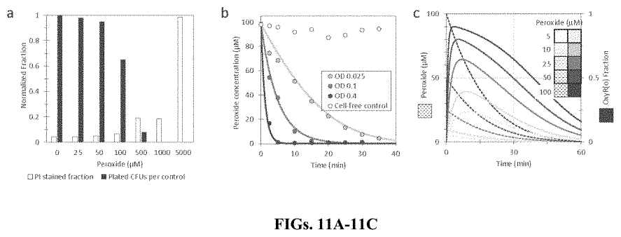

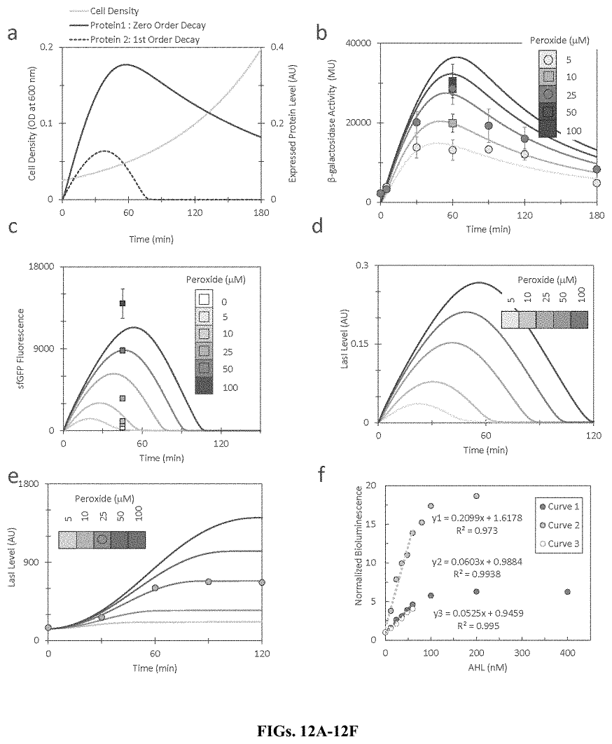

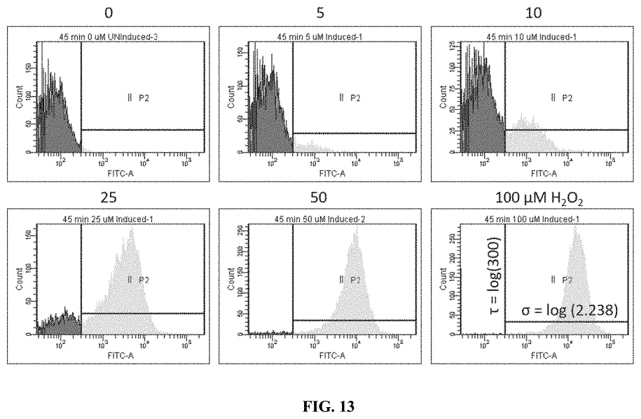

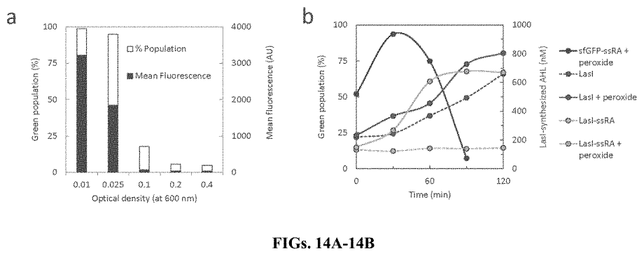

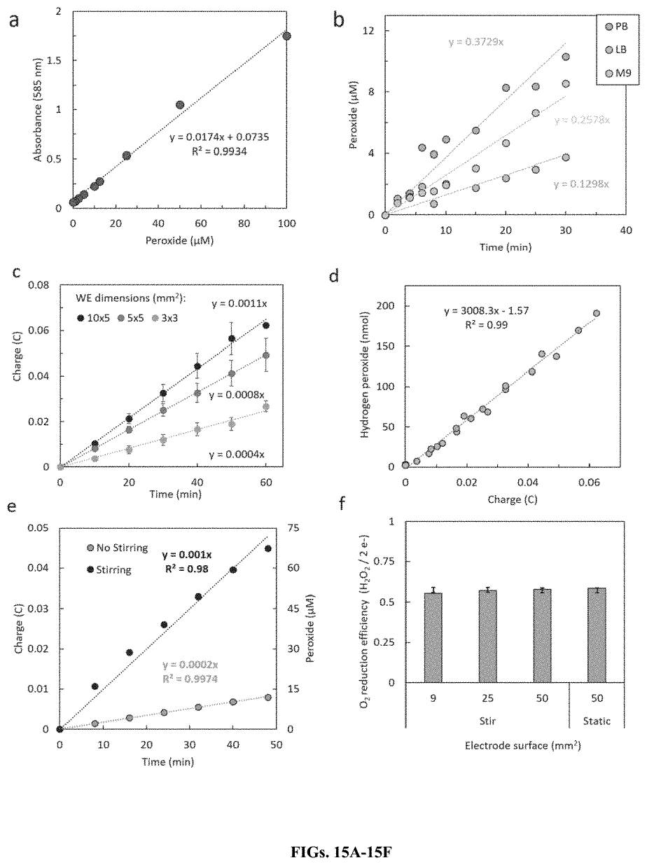

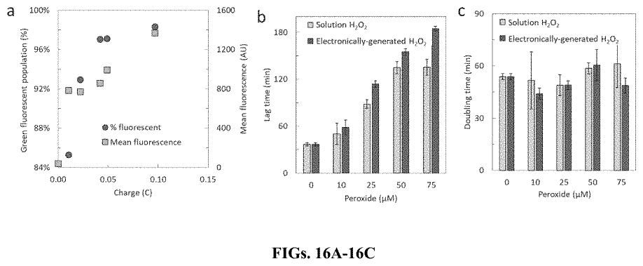

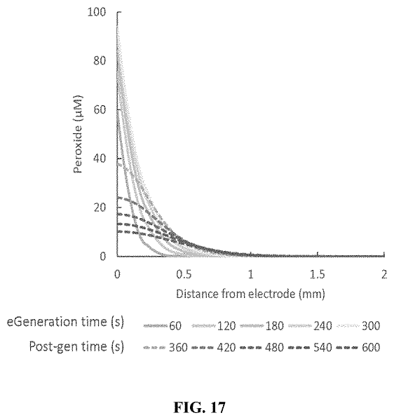

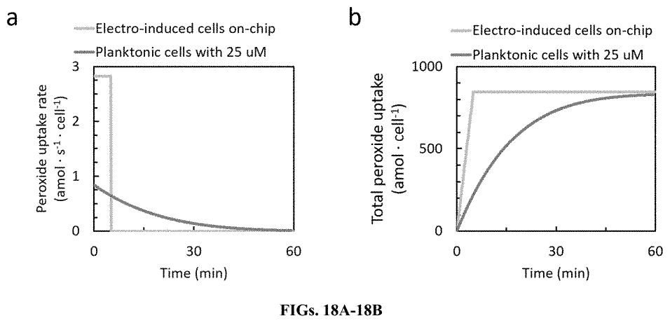

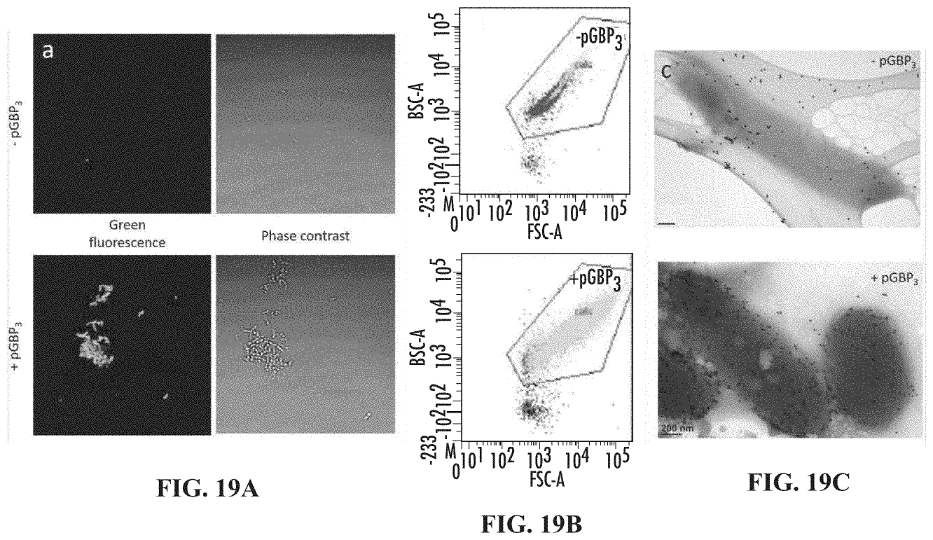

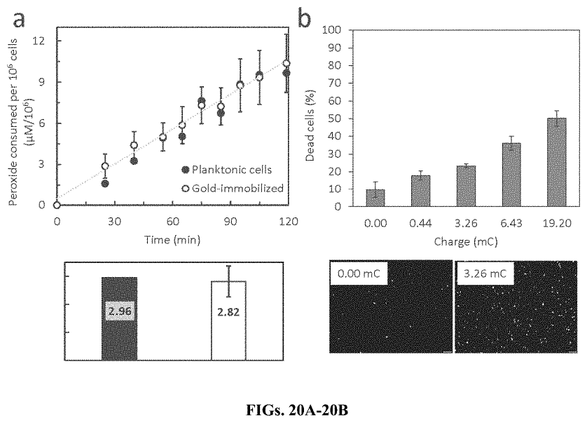

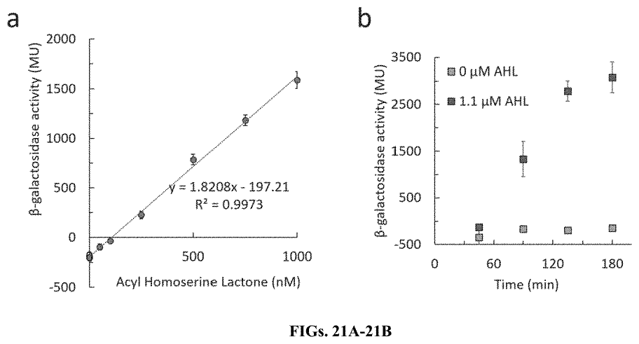

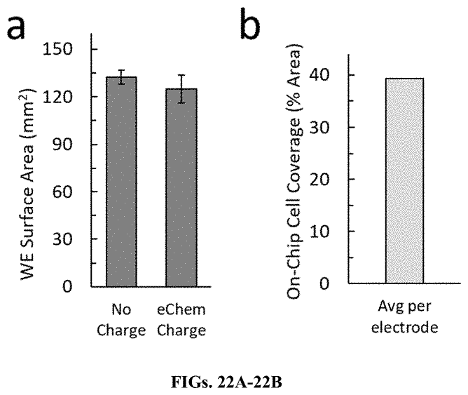

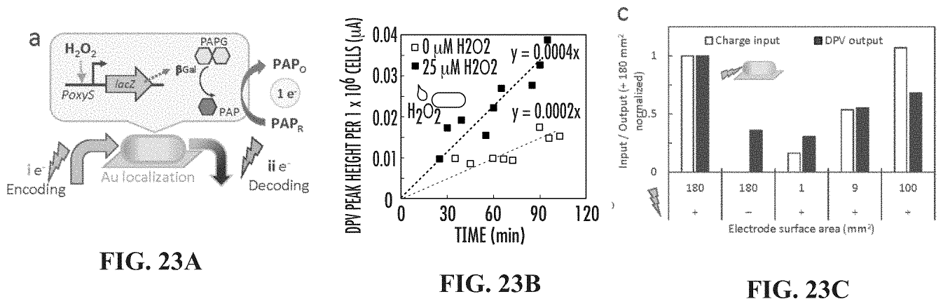

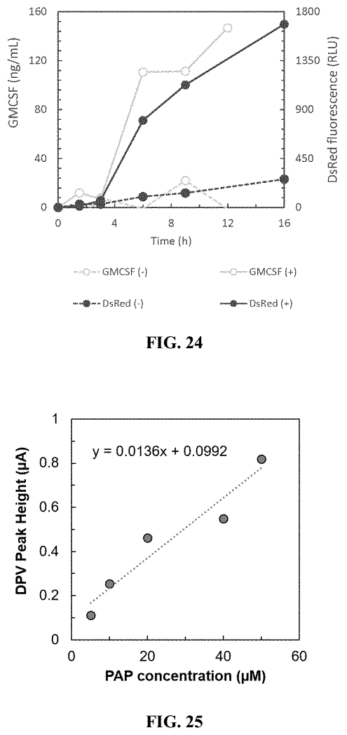

TABLE 6
Parameters for modeling peroxide flux.

| Term | Reference | Value |
|---|---|---|
| Coefficient of | USP-Technologies | 8.79 × |
| Peroxide Diffusion | (2019) ¹ | 10⁻⁶ cm² · s⁻¹ |
| Peroxide | 50 mm² WE Charge | 2.038 × 10⁻¹³ |
| generation rate | (C) = 0.0002 × | mol · mm⁻² · s⁻¹ |
| | Time (min) | |
| | FIG. 15C | |
| Immobilized | FIG. 8D | 67,000 per mm² |
| cell density | | |
| Cell unit peroxide consumption rate constant k_{qc} | $\frac{dC}{dt} = {k}_{qc} C ( t )$ FIG. 11C | 2 × 10⁻¹⁸ cell⁻¹ · s⁻¹ |
| Electrode unit | k_{qe} = k_{qc} | 1.34 × |
| peroxide | (Immobilized | 10⁻¹³ mm⁻² · s⁻¹ |
| consumption rate | cell density) | |
| constant k_{qe} | | |

### 1. OxyR-Based Electrogenetics for Bioelectronic Signal Transduction

To encode and transduce electronic information for cells, we developed an electrogenetic system using the native transcriptional activator, OxyR. E. coli rapidly uptakes and enzymatically degrades peroxide upon exposure, and its transient intracellular presence oxidizes OxyR (OxyR(o))^{22, 23}. This can elicit a strong native regulatory response, including upregulation of oxyS from the PoxyS promoter^{24, 25}. For electrogenetic system development, peroxide can be electrochemically generated from oxygen under physiological conditions²⁶.

For peroxide-driven gene induction, we harnessed an OxyR-regulated genetic circuit paired with constitutive OxyR expression²⁷ for peroxide-induced expression of genes of interest (sfGFP, lacZ, and lasI) from the PoxyS promoter (FIG. 6A). We found E. coli tolerated up to 100 μM of peroxide with negligible effect on cell viability. At these levels, near-saturated OxyR(o) levels were predicted and gene expression was characterized as a function of peroxide dose and time (see kinetic models, FIGS. 11A-11C, FIGS. 12A-12F, FIG. 13). Thus, in agreement with previous reports, 0-100 μM peroxide provided a benign induction range²⁸. Further, we found that OxyR was mostly oxidized (peak f_{OxyR(o)}˜0.6, FIG. 6B) leading to near-saturated expression levels. In analogous timecourse experiments with 25 μM peroxide, LasI-generated AHL levels and β-gal peaked 60 min post-induction (FIG. 6C), also in agreement with modeled kinetics. Overall, the consistency of modeled behavior with experimental data validated the OxyR kinetic predictions. This, along with induction dependence on cell number and protein degradation (FIGS. 14A-14B) provided design insights for further electrogenetic studies. We next induced OxyR-regulated gene expression via electronic input in lieu of exogenously-added peroxide. Peroxide can be electrochemically generated at physiological conditions (pH 7) and benign voltages (approximately −0.3 to −0.9 V vs. Ag/AgCl)²⁶ by the partial reduction of oxygen, according to Equation 1.

O₂+2H⁺+2e⁻↔H₂O₂ Equation (1)

Peroxide thus provides a transduction mechanism by which electronic input can be converted into a biologically-recognized cue in our system (FIG. 6D). Previous electrogenetics work coopted the SoxRS regulon, requiring purposely-added redox mediators to facilitate electron exchange between the electrode and nearby cells^{8, 18}. Here, no supplemental redox mediators are needed. For design studies, we biased an electrode at −0.4 V to reduce oxygen in amounts stoichiometrically proportional to applied charge. External influences on charge transfer impact the rate of peroxide generation, including electrode dimensions (FIG. 6E), media, and oxygen availability. Regardless, we found that the energy efficiency of oxygen reduction to peroxide was near-constant (59%, FIGS. 15A-15F). Applied charge, then, reliably represents a unit of “electro-induction dose” and, conveniently, has electrode area-based proportionality. We then tested electrogenetic induction by monitoring the β-gal activity of suspended cells using a 50 mm² surface. In FIG. 6F, activity increases over time and as a function of charge (current×time). All electro-induced samples exhibited activity above basal levels within 5 min. With a maximum dose, expression levels were on par with a 25 μM exogenously-induced control, which is indicative of having reached expression-saturating OxyR(o) levels. We saw similar charge-based induction of sfGFP and verified that growth behavior did not differ between electrochemically-produced and exogenously-supplied peroxide conditions (FIGS. 16A-16C). Thus, this novel electrogenetic circuit enables us to link direct electronic input, through redox transduction, to biological behavior.

B. 2. Elucidation of the Bioelectronic Interface

Electrode-generated peroxide in a quiescent fluid creates a far more conducive signaling environment compared to its exogenous addition to a planktonic culture, where peroxide must be uniformly mixed. We studied the spatiotemporal dynamics of each process to elucidate optimal conditions for uniform electrogenetic induction.

First, we simulated the peroxide gradient at the electrode in the presence of unstirred planktonic cells based on its generation rate, diffusion, and cellular consumption. The model data in FIG. 7A shows that peroxide reaches micromolar concentrations proximal to an electrode surface within seconds of a voltage bias. Despite significant peroxide consumption by cells (FIGS. 11B-11C), their presence as a planktonic population only marginally attenuates the bulk concentration (FIG. 17). Conversely, in a scenario where the entire cell population is distributed at the electrode surface, nearly all generated peroxide is intercepted, limiting its accumulation in bulk to 1.5 μM at its peak (FIG. 7B). Once peroxide generation ceases, the simulation shows an interfacial zone of complete elimination by the bacterial monolayer within 1 min.

We next analyzed the effect of cell position on OxyR dynamics. In FIGS. 7C-7D, we found that the cells' consumption near the electrode greatly limits the peroxide quantity available for distal cells, whereas all electrode-localized cells provide uniform consumption. For planktonic populations in the simulation, fewer than 7% of the cells experience maximal OxyR activation in a peroxide-rich environment (i.e. at least 25 μM peroxide whereby f_{OxyR(o)}≥0.6) near the electrode surface (denoted Z1). The peroxide concentration declines with distance, resulting in the majority of cells (82%) positioned furthest from the electrode (Z3), experiencing minimal peroxide levels (<4 μM) and hence, minimal OxyR activation (f_{OxyR(o)}≤0.1). By contrast, the fractions of activated OxyR and, consequently, of induced cells are higher for electrode-localized cells—achieving 0.9 f_{OxyR(o)} universally within 5 min of voltage bias (FIG. 7D).

Additionally, we note that these electro-induction parameters (i.e. immobilized configuration of the bioelectronics interface, 300 s voltage duration) yield net peroxide uptake nearly-equivalent to 25 μM induction (FIGS. 18A-18B), which was shown to sufficiently saturate protein expression (FIG. 6B). Thus, these design studies for in situ peroxide generation elucidate favorable conditions that should enable strong gene expression (e.g. for LasI-mediated AHL signaling) and, overall, provide a fast, efficient, and uniform bioelectronic information-transfer interface.

C. 3. Electrode-Immobilization of Electrogenetic Cells

In order to localize signal transfer at the bioelectronic interface, cells were engineered to enable direct assembly onto the electrode via peptide-mediated affinity interactions. We harnessed the outer membrane autotransporter pore-forming protein (AIDAc) as a vector for cell surface modification^{29, 30}. We fused AIDAc with a recombinant peptide consisting of a trimeric repeat of a non-natural peptide characterized for its high affinity to gold (GBP₃)³¹⁻³³. Our structural prediction of the GBP₃-AIDAc fusion (FIG. 8A) depicts the peptide's extrusion through the center of the AIDAc barrel as a relatively unstructured, extended conformation.

Surface-displayed expression of GBP₃ was initially tested by quantum dot (QD) labeling, which showed affinity for the GBP₃⁺ and not the GBP3⁻ cell surface, presumably due to the Zn-containing QD shell and the peptide's histidine tag through well-known affinity interactions with transition metals (FIGS. 19A-19C)³⁴. Furthermore, by transmission electron microscopy, gold nanoparticles did not associate with cells lacking GBP₃ (FIG. 8B), but showed retention on GBP₃⁺ cell surfaces (FIG. 8C). We found that GBP₃⁺ cells bound with fifty-fold higher specificity to planar-deposited gold compared to a silicon wafer's native oxide. Following this, representative composite fluorescence images show Syto9-stained cells immobilized on patterned gold electrodes with surface areas between 1 and 100 mm² (FIG. 8D). Cell distribution was visibly uniform and spatially defined by the electrode's geometry. Thus, one may potentially use electrode design to guide the electrogenetic signal transduction.

That is, we confirmed that gold-bound cells could be electro-induced to express sfGFP and LasI (via in situ electrochemical stimulus, FIG. 8E) while maintaining viability and statistically-similar peroxide consumption rates to those of planktonic cells (FIGS. 20A-20B). Overall, GBP₃ establishes the surface-assembled, physical connectivity that is important for efficient information flow across the bio-electronic interface.

### 4. Reflexive Verification of Signal Routing in a Bio-Network

We next aimed to (i) propagate the electrogenetic cue across multiple cell populations by redirecting native cell-to-cell communication and (ii) enable electrochemical verification of the signal transmission by producing an electrochemically detected redox-active output upon signal exchange. We accomplished this by networking two key cell populations, designated for signal routing and verification, respectively (FIG. 9A). First, the electrode-bound cells relay the original electronic signal by actuating an electrogenetic circuit designed to produce LasI-synthesized AHL that is routed via diffusion to the receiving network space. The diffusion space of AHL establishes network boundaries into which planktonic AHL-recipients can be connected, for instance, to verify signal strength throughout the network space. We created Verifier cells to respond to the routed signal providing electrochemical feedback, wherein their genetic responses reflected the received signal strength (FIGS. 21A-21B). Specifically, AHL-induced β-gal catalyzes production of electrochemically-active p-aminophenol (PAP), which is transduced into an electronic output by its electrochemical oxidation^{20, 35}. Provided that the electronic output reflects input status, continuity in information transfer across the bioelectronic interface is thus maintained.

We confirmed that AHL-sensing cells generated an electrochemically-detectable output based on β-gal-produced PAP. Differential pulse voltammetry (DPV) of AHL-induced cultures in a PAP-oxidizing voltage range (0.25 to −0.15 V) revealed a faster increase in peak current over time compared to uninduced samples (FIG. 9B). When electrogenetic AHL-producers were added with electro-induction, the co-cultures showed sustained and concomitant production of β-gal and electrochemical readout, both commensurate with charge dose (FIG. 9C). Of note, PAP accumulation over the culture period allows for continuous measurement without sample loss. Having confirmed that electronic input could be relayed from an electrogenetic router population to recipients, which validated charge input via electrochemical feedback, we assembled the Routers onto the electrode to improve signal efficiency. FIG. 9D portrays data for this configuration under ON and OFF conditions, where electro-induction occurred in an ON condition (i). Electrode sizes and representative electrode coverage by Router cells were similar between the two conditions (FIGS. 22A-22B). When ON, production of the biological signal, AHL, increased fifteen-fold (ii), attributed to Router activation. Additionally, AHL-induced β-gal expression in Verifier cells showed five-fold enhanced activity in ON samples (iii). Finally, the electronic output of ON cultures was approximately two-fold higher than the OFF sample output (iv).

With the signal-to-noise ratio (SNR) defined as the ratio of electrochemical readouts between ON and OFF samples, an SNR (FIG. 9E) of two in these experiments was found to be similar among the various system configurations (signal relayed on-chip, relayed planktonically, and a single-cell-type control, FIGS. 23A-23C). System robustness is demonstrated by noting that the SNR was consistent irrespective of the means of activation (electronic or chemical). The net result demonstrates a complete electronic-to-bio-to-electronic, and thus, bidirectional information exchange with a bacterial community.

D. 5. Coupling Bioelectronic Information Exchange to Actuation

By delocalizing electrogenetics from a discrete surface to a multi-population community through AHL communication, the bioelectronic system exploits native biological signaling processes to accommodate non-electrogenetic cell types. It follows that numerous and distinct populations could be networked in. For example, by the inclusion of AHL-responsive cells designated for executive functions as Actuators in parallel with electronic feedback from Verifier cells. We designed AHL-inducible Actuators with identical genetic control as Verifiers, so that Verifier output also indicates triggered Actuator function. In our examples, Actuators express and secrete protein products that might influence environments outside of the AHL-networked bioelectronics. Protein secretion enables a variety of actuation opportunities based on extracellular biomolecular recognition, interaction, or catalytic events. Here specifically, Actuator cells co-secrete a natively-recognized biotherapeutic peptide, granulocyte macrophage colony-stimulating factor (GMCSF) and DsRed as a fluorescent marker (FIG. 10A, FIG. 24). GMCSF has therapeutic efficacy for Crohn's disease; its secretion by E. coli has been previously accomplished at physiologically-relevant levels by co-expression of TolAIII pores.²¹ In conjunction, DsRed co-secretion provides optical reporting of the Actuators' AHL-responsive function.

We created consortia of varied compositions of Router, Verifier, and Actuator cells and probed for their diverse outputs (β-gal, extracellular DsRed and GMCSF) upon AHL signaling. The AHL-driven outputs strongly correlated with the respective population ratios (FIG. 10B), which demonstrates the diversity and resolution of AHL-networked functions. Hence, in addition to electrode size, charge dosage, and Router genetic circuitry, the population ratio is yet another tunable parameter for multiplexed consortium function.

To illustrate the fully-networked bioelectronics system, we established electrochemical connectivity by pairing electrode-bound Router cells with the co-culture of planktonic Verifier and Actuator cells. Samples were either maintained at an OFF state or turned ON (−3.4 mC, FIG. 10C (i)). To track the activity of the community, we measured AHL levels (ii), electrochemical output (iii), and product secretion levels (iv) throughout the incubation period. All of the outputs show similar trends for OFF vs ON samples. In OFF samples, AHL, PAP, DsRed, and GMCSF levels remained low throughout extended incubations. Conversely, in electronically-induced communities, measured outputs increased dramatically at 4 to 10-fold higher levels than those in OFF samples. All outputs showed differences between the ON and OFF conditions after 5.5 h and these grew over time. We note that the β-gal substrate supply at 20 h remained sufficient (<10% converted to PAP, FIG. 25) to extrapolate this trend beyond the experimental endpoint. This demonstrates for the first time bioelectronic I/O successfully directing and reporting distributed biological task execution in a three-member microbial community through an electrochemically-triggered and -read molecular information relay.

This particular consortium grouping is uniquely suited to function as a biological local area network (BioLAN) by bioelectronic information exchange between the electronic I/O and AHL-delineated coordination across Router, Verifier, and Actuator cells (FIG. 10D). Cell-to-cell communication as exemplified in this BioLAN, enables distribution of functions into a collective that is modular and multiplexed by cell type. Further, the consortium members expand the communication repertoire via electronic-transducing redox signals (peroxide, PAP), resulting in a convenient interfacing capability with electronics infrastructure. The electronic information flow can clearly be traced through induction current (FIG. 10E (i)) to the BioLAN output current (ii) to confirm system connectivity as real-time electronic feedback. This, in turn, informs on therapeutic secretion status due to AHL-correlated responses (FIG. 10E (ii)).

### II. Discussion

This work enables bioelectronic interfacing with a living biological network (BioLAN) by purposing redox molecules to interconvert between electronic and biological I/O to achieve bidirectional information exchange. Redox signal transduction provides “wiring” to connect to the synthetic biology, whose electronic proficiency is established by minimal genetic engineering. The engineered cells of the BioLAN have genetically distinct roles of signal routing, verification, and actuation and remain interconnected via internal AHL communication. The BioLAN “plugs into” the electronics infrastructure to seamlessly transmit information from electronics, through biology, and back out to electronics, and to concurrently drive programmed biological function.

As hydrogen peroxide is a universally-recognized redox molecule in biology that can be electrochemically-generated, we repurposed native oxidative stress regulation in the BioLAN for electrogenetic expression via the OxyR protein and PoxyS promoter. This newly-introduced system functions in aerobic environments and eliminates the need for exogenous mediators. Correlation between modeled peroxide-oxidized OxyR and charge input ensures that electrochemically-supplied peroxide is targeted at non-toxic, yet above-threshold levels for gene expression. Ultimately, voltage-mediated control over the electrogenetic response encodes the BioLAN's ON or OFF status. The electrogenetic cells serve as a Router component to distribute the information throughout the BioLAN. This is accomplished by system-specific signal propagation from the electrogenetic cells, which transduce the peroxide input signal into an orthogonal output, AHL. We note that AHL communication is orthogonal to the BioLAN—the microbes require non-native genetically engineered circuitry to recognize AHLs; programmed secretion of alternative molecules could accommodate broader signaling schemes. Native autoinducer AI-2, for example, would engage natural processes within the BioLAN³⁶. Furthermore, multiplexed signaling could independently target designated subpopulations for uniquely timed and carried out tasks.³⁷

Electrode localization of Router cells improves in situ induction kinetics and avoids diffusion limitations that can lead to heterogeneous responses in suspended biological systems³⁸, as confirmed by modeling OxyR redox state. Together, we show that the generation of an electrode-localized peroxide flux, its use for bio-electrochemical information transfer, and its pairing with the surface-attached cells, yields a robust, quasi-solid state electrogenetic platform. This setup invites future opportunities for scalable, spatially-programmable biological control via three-dimensional, soft, flexible, miniaturized and arrayed electrode formats for wearable, ingestible, or other portable systems³⁹⁻⁴². By combining this “hardwired” format with “wireless” signal propagation, one can envision AHL providing a high fidelity Routing capability that extends the network's boundaries to remotely-located AHL-responders, which could enable direct interfacing with ex situ environments in which a device is deployed.

Given that wireless network components typically return signals to confirm connectivity, the BioLAN includes reflexive feedback. That is, connectivity prompts AHL-responders to provide a verification signal directed to the central electronics. Here, Verifier cells inform on AHL-connectivity via PAP redox output; being mobile, they reflexively signal across the physical boundaries of the network with output signal amplitude as a real-time indicator of connectivity strength. In demonstrations, electronic feedback was measurable for 20+ hours post-induction, and provided ON/OFF system verification within 3 hours. Notably, each component of the BioLAN offers handles for further tuning of signal response times that contribute to the overall system timescale (Table 7). The combination of correlative dose-response relationships between charge inputs and outputs with gene expression demonstrated in this work and the added potential for optimizing system response times could feasibly support integrated feedback control for repeated dosing or precise dose adjustments.

Finally, the peripheral BioLAN components should perform electronically-programmed functions and thus enable remote bioelectronic actuation. Importantly, because Actuator cells homogenously co-populate the BioLAN with the Verifiers and utilize the same AHL-sensitive circuitry, the Verifiers' activity infers the status of co-occurring actuation events. We showed that release of the secreted DsRed and GMCSF payloads accumulated proportionally to the electrochemical readouts over time, validating the electronic output as a proxy of secretion status. Furthermore, actuation offers extended connectivity to external environments based on signal recognition: extracellular fluorescence (DsRed) as optical readout and a secreted biologic for potential in vivo interactions (GMCSF). As potential applications, secreted proteins could enable biorecognition and interactions for healthcare diagnostics and therapy⁴³, biomolecular self-assembly for biomaterial synthesis⁴⁴, or enzymatic catalysis for bioremediation⁴⁵.

Signal fidelity across all transduction formats was maintained and was tunable based on population ratio. In this way, the networking of multiple AHL-responsive cell types expands the influence of electrogenetic control, multiplexes outputs, and enables the electronic orchestration of complex tasks through signal relay, spatial segregation, and division of labor. While one could augment a single cell type to execute multiple functions, such as programming cells to exhibit both actuation and electronic reporting functions, their segmentation into separate populations highlights the potential for distributed networking. Areas such as bioconversion or biosynthesis have interest in such use of consortia.^{46, 47} Each subpopulation could be individually optimized and the network composition could be autonomously controlled for coordinated, collective function^{48, 49}.

In total, the established bidirectional dialogue represents a hybrid system with electronically-programmed and tracked, yet biologically-executed, functions. Because redox is both accessed electrochemically and serves as a medium of information exchange, this communication mode presents a reliable platform for “plugging in” the BioLAN. Moreover, because redox events are inherent within biology, the concepts shown here that couple electro-induction with biological signaling will enable biological connectivity to a variety of electronic devices (e.g., ingestible capsules, environmental sensors, electronic tattoos, etc)^{12, 50, 51}, where readily-implemented future developments will further augment BioLAN use for these applications. Bioelectronics connectivity featuring synthetic biology suggests a future Internet-of-BioNano-Things⁵², yielding applications capable of embedding “biological intelligence” in ecological settings, wearable interfaces, and in vivo environments.

TABLE 7
Timescales of signal transduction events.

| Mode of Signal | | | | |
|---|---|---|---|---|
| Transduction | Time | | Figure | Proposed Alterations for Tuning Timescale |
| Electro-generated peak peroxide | 4 | min | FIG. 17 | Change voltage magnitude from −0.4 V, |
| flux | | | | increase dissolved oxygen in liquid |
| Peak OxyR(o) due to electro- | 4 | min | FIG. 7D | Change exogenous peroxide concentration |
| induction | | | | or voltage-controlled flux; change |
| | | | | configuration between cells and electrode |
| | | | | surface |
| Peak sfGFP expression from | 45 | min | FIG. 8B | Change plasmid copy #, protein degradation |
| peroxide induction | | | | tag and/or expression of protease; genome |
| | | | | minimization |
| Peak β-galactosidase expression | 60 | min | FIG. 6C | Induce alpha complementation of β-galactosidase |
| from peroxide induction | | | | |
| Peak LasI expression from | 45 | min | FIG. 12E | Change plasmid copy #, protein degradation |
| peroxide induction | | | | tag and/or expression of protease; genome |
| | | | | minimization |
| Saturation of AHL signal level | 90 | min | FIG. 6C | Change LasI degradation rate and/or include |
| | | | | AHL degradation enzyme |
| Peak β-galactosidase expression | 180 min | | FIG. 21B | Change genetic regulation of LasR |
| from AHL induction | (3 h) | | | |
| Minimum time for electro- | 3 | h | FIG. 9B | Change regulation of cell machinery for |
| detection | | | | galactoside uptake (eg LacY) |
| Minimum time for TolAIII- | 6 | h | FIG. 24 | Change genetic regulation for TolAIII |
| mediated secretion | | | | expression, or use different export machinery |

### REFERENCES

1. Gubbi, J., Buyya, R., Marusic, S. & Palaniswami, M. Internet of Things (IoT): A vision, architectural elements, and future directions. Future Generation Computer Systems 29, 1645-1660 (2013).
2. Tian, B. et al. Roadmap on semiconductor-cell biointerfaces. Phys Biol 15, 031002 (2018).
3. Atkinson, J. T. et al. Metalloprotein switches that display chemical-dependent electron transfer in cells. Nat Chem Biol (2018).
4. Bird, L. J. et al. Engineered living conductive biofilms as functional materials. Mrs Commun 9, 505-517 (2019).
5. Shao, J. et al. Smartphone-controlled optogenetically engineered cells enable semiautomatic glucose homeostasis in diabetic mice. Sci Transl Med 9 (2017).
6. Mimee, M. et al. An ingestible bacterial-electronic system to monitor gastrointestinal health. Science 360, 915-918 (2018).
7. Yang, X. et al. Bioinspired neuron-like electronics. Nat Mater 18, 510-517 (2019).
8. Tschirhart, T. et al. Electronic control of gene expression and cell behaviour in Escherichia coli through redox signalling. Nat Commun 8, 14030 (2017).
9. Bhokisham, N. et al. A redox-based electrogenetic CRISPR system to connect with and control biological information networks. Nat Commun 11, 2427 (2020).
10. Sadat Mousavi, P. et al. A multiplexed, electrochemical interface for gene-circuit-based sensors. Nat Chem 12, 48-55 (2020).
11. Liu, Y. et al. Connecting Biology to Electronics: Molecular Communication via Redox Modality. Adv Healthc Mater 6 (2017).
12. Lee, H. et al. Wearable/disposable sweat-based glucose monitoring device with multistage transdermal drug delivery module. Sci Adv 3, e1601314 (2017).
13. Wang, S., Payne, G. F. & Bentley, W. E. in Gene Expression and Control (IntechOpen, 2018).
14. Green, J. & Paget, M. S. Bacterial redox sensors. Nat Rev Microbial 2, 954-966 (2004).
15. Hirose, A., Kouzuma, A. & Watanabe, K. Towards development of electrogenetics using electrochemically active bacteria. Biotechnol Adv 37, 107351 (2019).
16. McCarty, N. S. & Ledesma-Amaro, R. Synthetic Biology Tools to Engineer Microbial Communities for Biotechnology. Trends Biotechnol 37, 181-197 (2019).
17. Lindemann, S. R. et al. Engineering microbial consortia for controllable outputs. The Isme Journal 10, 2077 (2016).
18. Pomposiello, P. J. & Demple, B. Redox-operated genetic switches: the SoxR and OxyR transcription factors. Trends Biotechnol 19, 109-114 (2001).
19. Liu, Y. et al. Using a Redox Modality to Connect Synthetic Biology to Electronics: Hydrogel-Based Chemo-Electro Signal Transduction for Molecular Communication. Adv Healthc Mater 6 (2017).
20. VanArsdale, E. et al. Redox-Based Synthetic Biology Enables Electrochemical Detection of the Herbicides Dicamba and Roundup via Rewired Escherichia coli. ACS Sens 4, 1180-1184 (2019).
21. McKay, R. et al. A platform of genetically engineered bacteria as vehicles for localized delivery of therapeutics: Toward applications for Crohn's disease. Bioeng Transl Med 3, 209-221 (2018).
22. Zheng, M., Aslund, F. & Storz, G. Activation of the OxyR transcription factor by reversible disulfide bond formation. Science 279, 1718-1721 (1998).
23. Kim, S O. et al. OxyR: a molecular code for redox-related signaling. Cell 109, 383-396 (2002).
24. Aslund, F., Zheng, M., Beckwith, J. & Storz, G. Regulation of the OxyR transcription factor by hydrogen peroxide and the cellular thiol-disulfide status. Proc Natl Acad Sci USA 96, 6161-6165 (1999).
25. Demple, B. & Halbrook, J. Inducible repair of oxidative DNA damage in Escherichia coli. Nature 304, 466-468 (1983).
26. Sultana, S. T. et al. Electrochemical scaffold generates localized, low concentration of hydrogen peroxide that inhibits bacterial pathogens and biofilms. Sci Rep 5, 14908 (2015).
27. Rubens, J. R., Selvaggio, G. & Lu, T. K. Synthetic mixed-signal computation in living cells. Nat Commun 7, 11658 (2016).
28. Virgile, C. et al. Engineering bacterial motility towards hydrogen-peroxide. PLoS One 13, e0196999 (2018).
29. Hornstrom, D., Larsson, G., van Maris, A. J. A. & Gustaysson, M. Molecular optimization of autotransporter-based tyrosinase surface display. Biochim Biophys Acta Biomembr 1861, 486-494 (2019).
30. Terrell, J. L. et al. Nano-guided cell networks as conveyors of molecular communication. Nat Commun 6, 8500 (2015).
31. Brown, S. Metal-recognition by repeating polypeptides. Nat Biotechnol 15, 269-272 (1997).
32. Tamerler, C. et al. Materials specificity and directed assembly of a gold-binding peptide. Small 2, 1372-1378 (2006).
33. Verde, A. V., Acres, J. M. & Maranas, J. K. Investigating the specificity of peptide adsorption on gold using molecular dynamics simulations. Biomacromolecules 10, 2118-2128 (2009).
34. Medintz, I. L. et al. Quantum-dot/dopamine bioconjugates function as redox coupled assemblies for in vitro and intracellular pH sensing. Nat Mater 9, 676-684 (2010).
35. Tschirhart, T. et al. Electrochemical Measurement of the beta-Galactosidase Reporter from Live Cells: A Comparison to the Miller Assay. ACS Synth Biol 5, 28-35 (2016).
36. DeLisa, M. P., Wu, C. F., Wang, L., Valdes, J. J. & Bentley, W. E. DNA microarray-based identification of genes controlled by autoinducer 2-stimulated quorum sensing in Escherichia coli. J Bacteriol 183, 5239-5247 (2001).
37. Meyer, A. J., Segall-Shapiro, T. H., Glassey, E., Zhang, J. & Voigt, C. A. Escherichia coli “Marionette” strains with 12 highly optimized small-molecule sensors. Nature Chemical Biology 15, 196-204 (2019).
38. Servinsky, M. D. et al. Directed assembly of a bacterial quorum. ISMS J 10, 158-169 (2016).
39. Shang, W. et al. Selective assembly and functionalization of miniaturized redox capacitor inside microdevices for microbial toxin and mammalian cell cytotoxicity analyses. Lab Chip 18, 3578-3587 (2018).
40. Pennacchio, F. A., Garma, L. D., Matino, L. & Santoro, F. Bioelectronics goes 3D: new trends in cell-chip interface engineering. Journal of Materials Chemistry B 6, 7096-7101 (2018).
41. Yuk, H., Lu, B. & Zhao, X. Hydrogel bioelectronics. Chem Soc Rev (2018).
42. Kang, M. et al. Signal processing approach to probe chemical space for discriminating redox signatures. Biosens Bioelectron 112, 127-135 (2018).
43. Hwang, I. Y. et al. Engineered probiotic Escherichia coli can eliminate and prevent Pseudomonas aeruginosa gut infection in animal models. Nat Commun 8, 15028 (2017).
44. Sun, F., Zhang, W.-B., Mandavi, A., Arnold, F. H. & Tirrell, D. A. Synthesis of bioactive protein hydrogels by genetically encoded SpyTag-SpyCatcher chemistry. Proceedings of the National Academy of Sciences 111, 11269 (2014).
45. French, K. E., Zhou, Z. & Terry, N. Horizontal ‘gene drives’ harness indigenous bacteria for bioremediation. Scientific Reports 10, 15091 (2020).
46. Jiang, Y., Dong, W., Xin, F. & Jiang, M. Designing Synthetic Microbial Consortia for Biofuel Production. Trends in Biotechnology 38, 828-831 (2020).
47. Jawed, K., Yazdani, S. S. & Koffas, M. A. G. Advances in the development and application of microbial consortia for metabolic engineering. Metabolic Engineering Communications 9, e00095 (2019).
48. Alper, H. S. & Avalos, J. L. Metabolic pathway engineering. Synthetic and systems biotechnology 3, 1-2 (2018).
49. Stephens, K., Pozo, M., Tsao, C. Y., Hauk, P. & Bentley, W. E. Bacterial co-culture with cell signaling translator and growth controller modules for autonomously regulated culture composition. Nat Commun 10, 4129 (2019).
50. Beardslee, L. A. et al. Ingestible Sensors and Sensing Systems for Minimally Invasive Diagnosis and Monitoring: The Next Frontier in Minimally Invasive Screening. ACS Sensors 5, 891-910 (2020).
51. Windmiller, J. R. et al. Electrochemical sensing based on printable temporary transfer tattoos. Chemical Communications 48, 6794-6796 (2012).
52. Akyildiz, I. F., Pierobon, M., Balasubramaniam, S. & Koucheryavy, Y. The Internet of Bio-Nanothings. Ieee Commun Mag 53, 32-40 (2015).
53. Kelley, L. A., Mezulis, S., Yates, C. M., Wass, M. N. & Sternberg, M. J. The Phyre2 web portal for protein modeling, prediction and analysis. Nat Protoc 10, 845-858 (2015).
54. Terrell, J. L. et al. Investigation of engineered bacterial adhesins for opportunity to interface cells with abiotic materials. Proc Spie 9863 (2016).
55. Rhoads, M. K. et al. Modification and Assembly of a Versatile Lactonase for Bacterial Quorum Quenching. Molecules (Basel, Switzerland) 23 (2018).
56. Lindsay, A. & Ahmer, B. M. M. Effect of sdiA on biosensors of N-acylhomoserine lactones. Journal of bacteriology 187, 5054-5058 (2005).
57. Wang, L., Hashimoto, Y., Tsao, C.-Y., Valdes, J. J. & Bentley, W. E. Cyclic AMP (cAMP) and cAMP receptor protein influence both synthesis and uptake of extracellular autoinducer 2 in Escherichia coli. Journal of bacteriology 187, 2066-2076 (2005).

1. A method of producing a recombinant amino acid in one or a plurality of bacterial cells in a culture vessel comprising a solid substrate, said method comprising:
(a) contacting a first bacterial cell or a first population of isolated bacterial cells with the solid substrate, said substrate comprising at least one exterior surface, at least one interior surface and at least one interior chamber defined by the interior surface and at least one opening;
(b) applying a cell medium into the culture vessel with a volume of cell medium sufficient to cover the at least one interior chamber; and
(c) exposing the first bacterial cells or first population with an inducer for a time period sufficient to stimulate expression of the recombinant amino acid;
wherein the first cell or first population of cells comprises a nucleic acid molecule comprising an expressible nucleic acid sequence encoding the amino acid operably linked to a regulatory sequence specific for association with the inducer.

2. The method of claim 1, wherein step (a) is preceded by transforming the first bacterial cell or first population of bacterial cells with the nucleic acid sequence.

3. The method of claim 1, wherein the solid substrate comprises a base with a predetermined shape that defines the shape of the exterior and interior surface.

4. The method of claim 1, wherein the solid substrate comprises one or a combination of silica, plastic, ceramic, or metal and wherein the base is in a shape of a cylinder or rectangular prism or in a shape substantially similar to a cylinder or a rectangular prism, such that coat the interior surface of the base and define a cylindrical or substantially cylindrical interior chamber or compartment; and wherein the opening is positioned at one end of the cylinder.

5. The method claim 1, wherein the inducer is peroxide, hydrogen peroxide, an oxidized form of OxyR or autoinducer 1.

6. The method of claim 1, wherein the nucleic acid molecule is free of secA, an extracellular secretion tag, and/or an outer membrane protein.

7. The method of claim 1, wherein the culture vessel further comprises a second population of bacterial cells; or a second and third population of cells.

8. The method of claim 1, wherein the first bacterial cell or the first population of cells comprises a second expressible nucleic acid sequence encoding OxyR, wherein the nucleic acid sequence encoding OxyR operably linked to a proD promoter sequence.

9. The method of claim 1, wherein, upon exposure to an inducer, the first bacterial cell or first population of bacterial cells stimulates expression of a cytokine.

10. The method of claim 1, wherein the solid substrate comprises at least one electrode or is positioned within about 10 millimeters from the solid substrate, and the method further comprises a step of exposing the electrode to voltages from about 0.1 to about 1.8 Volts.

11. The method of claim 1, wherein the method is performed without exposure to reduction-oxidation mediators.

12. The method of claim 1, wherein the solid substrate comprises a first, second and third vessel, each vessel of a size and shape sufficient to allow diffusion of protein, nutrients, and oxygen through the solid substrate in the presence of the cell culture medium; and wherein the first, second and third vessel each comprise a first, second, and third population of bacterial cells, respectively.

13. The method of claim 12, wherein the first, second and third vessels are in fluid communication.

14. The method of claim 1 further comprising the step of exposing the culture vessel to 37° Celsius and a level of carbon dioxide of no more than about 5.0% for a time sufficient to allow expression of the amino acid in the first cell or first population of cells in the interior chamber.

15. The method of claim 1, wherein the first bacterial cell or first population of bacterial cells are a non-pathogenic strain of bacteria from Escherichia.

16. A system comprising:
(i) a solid substrate comprising at least a first vessel, wherein the first vessel comprises a first bacterial cell or a first population of bacterial cells;
(ii) an electrode positioned on or proximate to the solid substrate;
(iii) a cell culture medium;
wherein the solid substrate comprises at least one electrode or is positioned within about 10 millimeters from the first vessel; and wherein the first bacterial cell or population comprises at least a first and a second nucleic acid sequence, the first nucleic acid sequence comprising at least one non-constitutive promoter operably linked to the second nucleic acid sequence; the second nucleic acid encoding: (a) at least one therapeutic agent; or (b) a signaling molecule;
wherein the non-constitutive promoter is an inducible promoter responsive to at least one stimuli, and the at least one stimuli comprises: (x) the presence of a certain density or a certain number of bacterial cells comprising the first and second nucleic acid sequences; or (y) the presence of an inducer.

17. The system of claim 16, wherein the system is in operable connection to at least one computer storage memory.

18. The system of claim 16 further comprising a digital display in operable connection to the at least one electrically conductive material by an electrical circuit capable of carrying an a electrical signal corresponding to a quantity of ion concentration in the first to the digital display, wherein the digital display is a configured to display concentration value of ion concentration and/or an amount of amino acid in a sample when the at least one electrically conductive material is in contact with the volume of the first vessel for a time period sufficient for an inducer to induce release of the amino acid sequence from the first bacterial cell or first bacterial population of cells.

19. The system of claim 16, wherein the first bacterial cell or first population of bacterial cells are non-pathogenic bacterial cells is chosen from one or a combination of the genera chosen from: Salmonella, Escherichia, Firmicutes, Bacteroidetes, Lactobacillus, Bifidobacteria, or Acidopholus.

20. The system of claim 16, wherein the bacterial cell comprises no more than five expressible exogenous nucleic acid sequences that are coding sequences, wherein the first exogenous nucleic acid sequence comprises at least one non-constitutive promoter operably linked to the second exogenous nucleic acid sequence that encodes the at least one therapeutic agent.

21. The system of claim 20, wherein the therapeutic agent is a cytokine.

22. The system of claim 17 further comprising a computer processor in operable connection with the at least one computer storage memory.

23. The system of claim 16, wherein the inducer is peroxide, hydrogen peroxide, autoinducer 1, or a derivative thereof.

24. The system of claim 16, wherein the first bacterial cell or first population of bacterial cells are free of an exogenous nucleic acid sequence that encodes one or a combination of secA, an extracellular secretion tag, or an outer membrane protein (Omp).

25. The system of claim 16 further comprising a second population of bacterial cells; or a second and third population of bacterial cells; each of the second and/or third population of bacterial cells comprising at least one nucleic acid molecule that comprises an expressible nucleic acid sequence encoding a signaling molecule operably linked to a regulatory sequence responsive to a recombinant product of at least one of the other bacterial cells or bacterial cell populations.

26. The system of claim 16, wherein the first bacterial cell or the first population of cells comprises a second expressible nucleic acid sequence encoding OxyR, wherein the nucleic acid sequence encoding OxyR operably linked to a PoxyS promoter sequence.

27. The system of claim 16, wherein the electrode is positioned within about 10 millimeters from the first bacterial cell or first bacterial cell population and wherein the system is free of a reduction-oxidation mediator.

28. The system of claim 16, wherein the first bacterial cell or first bacterial cell population is immobilized to the first vessel via an interaction between a protein on the bacterial cell surface and a metal coating on the surface of the first vessel.

29. The system of claim 16, wherein the signal-to-noise ratio of the system is from about 1.8 to about 2.3.

30. A method of inducing electrostimulative release of a signaling molecule or therapeutic protein from a bacterial cell comprising:
(a) growing one or more bacterial cells of a first population of cells in the system of claim 16;
(b) introducing one or more stimuli to the one or more bacterial cells.

31. The method of claim 30 further comprising a step of measuring one or more responses from the one or more bacterial cells to the one or more stimuli based upon a product of an oxidation or reduction reaction conducted in the first vessel.

32. A method of selective secretion of signaling molecule or amino acid sequence in a first population of cells comprising:
(a) growing one or more bacterial cells in culture;
(b) introducing one or more stimuli to the one or more bacterial cells; wherein the one or more stimuli comprises exposing the one or more bacterial cells to: (i) a magnitude of electrical voltage or electrical current; or (ii) a density or amount of bacterial cells sufficient to cause selective release of the signaling molecule or amino acid sequence.

33. The method of claim 32, wherein the one or more bacterial cells are at least a portion of the first cell population in the system of claim 16.

34. The method of claim 32, wherein the signaling molecule or amino acid sequence is a green fluorescent protein, a cytokine, AHL, beta-galactosidase, LacI gene product or DsRed.

35. The method of claim 32, wherein the method is free of a step of lysing the one or more bacterial cells through exposure of enzyme that lyses the bacterial cells.
